SEQUENCE LISTING (2 sequences)

SEQ ID NO: 1 - protein, 8 aa, Unknown
  Description of Unknown NEB exon biomarker insertion sequence
WKYTKEQN

SEQ ID NO: 2 - DNA, 11 bp, Unknown
  Description of Unknown NEB exon biomarker insertion sequence
ggaaatatact

(19) United States
(12) Patent Application Publication
(10) Pub. No.: US 2018/0119137 A1
(43) Pub. Date: May 3, 2018

(54) INTEGRATED SYSTEMS AND METHODS FOR AUTOMATED PROCESSING AND ANALYSIS OF BIOLOGICAL SAMPLES, CLINICAL INFORMATION PROCESSING AND CLINICAL TRIAL MATCHING

(71) Applicant: Driver, Inc., San Francisco, CA (US)

(72) Inventors: Tetsuya Matsuguchi, San Francisco, CA (US); John Alden St. John, San Francisco, CA (US); Evangelos Pazarentzos, San Francisco, CA (US); William Polkinghorn, San Francisco, CA (US); Petros Giannikopoulos, San Francisco, CA (US)

(21) Appl. No.: 15/727,501

(22) Filed: Oct. 6, 2017

Related U.S. Application Data
(63) Continuation of application No. PCT/US17/52956, filed on Sep. 22, 2017.
(60) Provisional application No. 62/480,307, filed on Mar. 31, 2017.
(60) Provisional application No. 62/399,221, filed on Sep. 23, 2016.

(51) Int. Cl. C12N 15/10; C12Q 1/6874; C12Q 1/6886

(52) U.S. Cl. CPC C12N 15/1072; C12Q 2600/156; C12Q 1/6886; C12Q 1/6874

(57) ABSTRACT

The present disclosure provides a method for identifying a genomic aberration in one or more biological samples of a subject. The biological samples may be obtained and may comprise a nucleic acid sample that has or is suspected of having one or more genomic aberration(s) that appears at a frequency of less than about 5% in the nucleic acid sample. The nucleic acid sample may be enriched for a plurality of nucleic acid sequences to provide an enriched nucleic acid sample using a probe set comprising probes that have an on-target rate as a group of at least about 80%. Next, the enriched nucleic acid sample may be sequenced to generate sequencing reads. The sequencing reads can be processed to identify genomic aberration(s) in the one or more biological samples of the subject that appears at a frequency of less than about 5% in the nucleic acid sample.

### CROSS-REFERENCE

The present application is a continuation of International Application No. PCT/US17/52956, filed Sep. 22, 2017, which claims the benefit of U.S. Provisional Patent Application Ser. No. 62/399,221, filed Sep. 23, 2016 and U.S. Provisional Patent Application Ser. No. 62/480,307, filed Mar. 31, 2017, each of which is entirely incorporated herein by reference.

### BACKGROUND

Early detection and monitoring of diseases may be useful in a number of diagnostic methods. Mutations may be detected in associations with establishing a higher risk of a disease for a patient. Disorders can be a result of changes in epigenetic markers or rare genetic alterations. Such disorders may be characterized with DNA and RNA sequence information. In some cases, the disease may be identified and characterized by biological markers, such as nucleotide insertions and deletions, nucleotide substitutions, amino acid insertions, amino acid deletions, amino acid substitutions, gene fusions, copy-number variations, translocations, or gene expression signatures.

In the past, patients with a particular disease may be identified and enrolled into clinical trials from an investigator's clinic or practice from advertising or referrals. The clinical trials may be paper-based, unavoidably burdensome, slow to monitor, process, and store. In addition, with pharmaceutical companies producing more novel drug compounds, it is important for pharmaceutical companies to test and market new drugs in a minimum amount of time. Embodiments of the invention provide methods for analyzing a biological sample of a subject, identifying a disease in a subject, and using a computer implemented method to extract clinical history and data from a biological sample for clinical trial enrollment and drug development.

### SUMMARY

In certain aspects, the disclosure provides a method for qualifying a subject for a subset of therapies comprising clinical trials or standard of care treatments for one or more types of cancers, comprising: (a) subjecting at least one biological sample from the subject to at least one assay to generate biologic data from the subject; (b) processing the biologic data from the subject against a filtered set of therapies to generate the subset of therapies for which the subject qualifies, wherein the subset of therapies comprises the clinical trials or standard of care treatments for the one or more types of cancers, which filtered set of therapies is generated by computer assessing eligibility of a database of therapies against one or more criteria; and (c) presenting the subset of therapies on a user interface on an electronic device of a user. In certain embodiments, the method for qualifying a subject further comprises transmitting medical history data of the subject to one or more therapy coordinators of the subset of therapies.

In certain embodiments, the method for qualifying a subject further comprises receiving a selection from the subject as to a given clinical trial from the subset of therapies. In certain embodiments, the method for qualifying a subject further comprises receiving a request for enrollment of the subject in a therapy selected from the subset of therapies through the user interface. In certain embodiments, the method for qualifying a subject further comprises computer assessing the eligibility of the database of therapies against the one or more criteria to generate the filtered set of therapies. In certain embodiments, computer assessing the eligibility comprises (i) identifying at least one portion of the database of therapies; and (ii) curating at least one portion of the database of therapies using one or more clinical labels or molecular labels to generate the filtered set of therapies. In certain embodiments, the user interface comprises one or more graphical elements with one or more network links to the subset of therapies and contact information for the subset of therapies for which the subject qualifies. In certain embodiments, the subset of therapies comprises clinical trials or standard of care treatments for one or more types of cancers. In certain embodiments, the biologic data is generated from at least one biological sample of the subject by an automated assaying system, which automated assaying system uses automated processing for at least one member selected from the group consisting of cell extraction, nucleic acid extraction, enrichment, sequencing, and immunohistochemistry, during processing of at least one biological sample. In certain embodiments, step (b) comprises validating the filtered set of therapies by a human therapy curator. In certain embodiments, step (b) further comprises using medical history data of the subject to generate the subset of therapies for which the subject qualifies, wherein the medical history data is separate from the biologic data. In certain embodiments, the medical history data is identifiable according to medical text segments from the medical history data of the subject. In certain embodiments, the method for qualifying a subject further comprises using at least one machine learning algorithm to detect and label the medical text segments. In certain embodiments, step (b) comprises validating the subset of therapies for which the subject qualifies by a human therapy curator. In certain embodiments, at least one biological sample comprises a tumor tissue sample or a blood sample. In certain embodiments, the method for qualifying a subject further comprises, prior to step (a), (i) receiving a first nucleic acid sample from a tumor sample of the subject; and (ii) receiving a second nucleic acid sample from a normal sample of the subject. In certain embodiments, the method for qualifying a subject further comprises enriching the first nucleic acid sample for a plurality of nucleic acid sequences to provide an enriched nucleic acid sample using a probe set comprising probes that have an on-target rate as a group of at least about 80%, as determined by (i) measuring, for the probe set in at least one predetermined region, (1) probe coverage of each probe in the probe set and (2) off-target probe coverage for each probe in the probe set, and (ii) determining the on-target rate of the probe set based on a ratio of the off-target coverage to the probe coverage. In certain embodiments, the method for qualifying a subject further comprises assaying the enriched nucleic acid sample and the second nucleic acid sample to identify one or more genomic aberrations in a biological sample to generate the biologic data for the subject. In certain embodiments, the method for qualifying a subject further comprises labeling one or more genomic aberrations in the biological sample.

In certain aspects, the disclosure provides a method for qualifying a subject for a subset of therapies, comprising: (a) receiving medical history data and biologic data for the subject wherein the biologic data is generated from one or more biological samples of the subject;(b) computer analyzing the medical history data and the biologic data to yield a genomic-based medical history analysis for the subject; (c) using the genomic-based medical history analysis for the subject to query one or more databases of therapies for the subject, to generate the subset of therapies for which the subject qualifies; and (d) providing the subset of therapies on a user interface on an electronic device of a user.

In certain embodiments, the biologic data is generated from one or more biological samples of the subject by an automated assaying system, which automated assaying system uses automated processing for at least one member selected from the group consisting of cell extraction, nucleic acid extraction, enrichment, sequencing, and immunohistochemistry. In certain embodiments, the method for qualifying a subject further comprises computer assessing eligibility of the one or more databases of therapies against one or more criteria to generate a filtered set of therapies. In certain embodiments, the one or more databases is computer assessed using medical history data. In certain embodiments, the genomic-based medical history analysis for the subject comprises labels from the medical history data and labels from the biologic data, and wherein (c) comprises computer processing the labels against therapies from one or more database to yield the subset of therapies for which the subject qualifies. In certain embodiments, the method for qualifying a subject further comprises receiving a selection from the subject as to a given therapy from the subset of therapies. In certain embodiments, the method for qualifying a subject further comprises receiving a request for enrollment of the subject in a therapy selected from the provided subset of therapies through the user interface. In certain embodiments, the user interface comprises one or more graphical elements with one or more network links to the subset of therapies and contact information for the subset of therapies for which the subject qualifies. In certain embodiments, the subset of therapies comprises clinical trials or standard of care treatments for one or more types of cancers. In certain embodiments, step (c) comprises validating the subset of therapies for which the subject qualifies by a human therapy curator. In certain embodiments, prior to the step (a) the method comprises (i) receiving a first nucleic acid sample from a tumor sample of the subject; and (ii) receiving a second nucleic acid sample from a normal sample of the subject. In certain embodiments, the method for qualifying a subject further comprises enriching the first nucleic acid sample for a plurality of nucleic acid sequences to provide an enriched nucleic acid sample using a probe set comprising probes that have an on-target rate as a group of at least about 80%, as determined by (i) measuring, for the probe set in at least one predetermined region, (1) probe coverage of each probe in the probe set and (2) off-target probe coverage for each probe in the probe set, and (ii) determining the on-target rate of the probe set based on a ratio of the off-target coverage to the probe coverage. In certain embodiments, the method for qualifying a subject further comprises assaying the enriched nucleic acid sample and the second nucleic acid sample to identify one or more genomic aberrations in a biological sample to generate biologic data for the subject. In certain embodiments, prior to step (b), the medical history data is processed and transformed to provide processed medical history data. In certain embodiments, processing is selected from the group consisting of cleaning, organizing, and labeling. In certain embodiments, the subset of therapies comprises clinical trials or standard of care treatments for one or more types of cancer.

In certain embodiments, the method for qualifying a subject further comprises presenting the subset of therapies to a clinician to select for a recommended therapy. In certain embodiments, the method for qualifying a subject further comprises receiving a selection from the subset of therapies from the clinician. In certain embodiments, the biologic data include nucleic acid mutations or differentially expressed proteins. In certain embodiments, the nucleic acid mutations are selected from genes and variants of Table 1. In certain embodiments, (c) comprises querying one or more databases for one or more targeted therapies according to a predetermined gene or genomic region. In certain embodiments, the subset of therapies in (c) excludes therapies that target genomic aberrations absent in the biologic data. In certain embodiments, (c) comprises removing therapies that target genomic aberrations absent in the biologic data. In certain embodiments, the subset of therapies in (c) is filtered according to clinical phases of the therapy. In certain embodiments, the medical history data is identifiable according to medical text segments from the medical history data of the subject. In certain embodiments, the method for qualifying a subject further comprises using at least one machine learning algorithm to detect and label the medical text segments. In certain embodiments, (c) comprises determining ineligible therapies according to a categorical score and rejecting the ineligible therapies from remaining therapies to generate the subset of therapies. In certain embodiments, the categorical score is selected from the group consisting of yes, maybe, and no. In certain embodiments, the subset of therapies are compared and reviewed. In certain embodiments, the subset of therapies is passed to a user to manually verify eligibility using links to information from the medical history data and the biologic data for the subject.

In certain embodiments, the method for qualifying a subject further comprises filtering the subset of therapies based on filtering preferences of the user. In certain embodiments, filtering further comprises an evaluation by a healthcare professional and a selection for a recommended therapy. In certain embodiments, the subset of therapies is generated from one or more databases of therapies without use of the biologic data of the subject. In certain embodiments, step (a) comprises receiving phenotype information for the subject. In certain embodiments, the method for qualifying a subject further comprises (e) monitoring the subject enrolled in the subset of therapies by assaying one or more biological samples from the subject, wherein assaying is directed to 100 or more genes or variants thereof selected from Table 1. In certain embodiments, the querying of step (c) has a predicted likelihood of matching to a clinical trial of at least about 90%. In certain embodiments, the one or more biological samples are assayed for a presence or absence of biological markers at a concordance correlation coefficient of greater than or equal to about 90% when the one or more biological samples is re-assayed for the presence or absence of the biological markers, which biological markers include a plurality of different types of biological markers. In certain embodiments, the assaying covers at least 2,500 genes, gene fusions, point mutations, indels, copy-number variations, promoters, or enhancers. In certain embodiments, the subject is diagnosed with a solid tumor or cancer. In certain embodiments, the biologic data generates an initial list of therapies and the medical history data filters the initial list of therapies to generate the subset of therapies.

In certain aspects, the disclosure provides a method for qualifying a subject for a subset of therapies, comprising: (a) receiving (i) a first nucleic acid sample from the subject, which first nucleic acid sample has or is suspected of having tumor-derived cells or biological markers, and (ii) a second nucleic acid sample from a normal sample of the subject; (b) enriching the first nucleic acid sample for a plurality of nucleic acid sequences to provide an enriched nucleic acid sample using a probe set comprising probes that have an on-target rate as a group of at least about 80%, as determined by (i) measuring, for the probe set in at least one predetermined region, (1) probe coverage of each probe in the probe set and (2) off-target probe coverage for each probe in the probe set, and (ii) determining the on-target rate of the probe set based on a ratio of the off-target coverage to the probe coverage; (c) assaying the enriched nucleic acid sample and the second nucleic acid sample to identify one or more genomic alterations in the first nucleic acid sample relative to the second nucleic acid sample to generate a set of genomic data for the subject; (d) querying one or more databases of therapies for one or more therapies corresponding to a medical history of the subject and the genomic data, to generate the subset of therapies for which the subject qualifies; and (e) providing the subset of therapies on a user interface on an electronic device of a user.

In certain embodiments, the method for qualifying a subject further comprises receiving a selection from the subject as to a given therapy from the subset of therapies. In certain embodiments, the method for qualifying a subject further comprises receiving a request for enrollment of the subject in a therapy selected from the subset of therapies through the user interface. In certain embodiments, the method for qualifying a subject further comprises computer assessing eligibility of the one or more databases of therapies against one or more criteria to generate a filtered set of therapies. In certain embodiments, the user interface comprises one or more graphical elements with one or more network links to the subset of therapies and contact information for the subset of therapies for which the subject qualifies.

In certain embodiments, the subset of therapies comprises clinical trials or standard of care treatments for one or more types of cancers. In certain embodiments, step (d) comprises validating the subset of therapies for which the subject qualifies by a human therapy curator. In certain embodiments, the method for qualifying a subject further comprises receiving medical history data for the subject. In certain embodiments, the method for qualifying a subject further comprises identifying a therapeutic target based on the medical history and the genomic data and enrolling the subject in a therapy based on the identified therapeutic target. In certain embodiments, the method for qualifying a subject further comprises monitoring the subject, the monitoring comprising assaying one or more nucleic acid samples to generate genomic data, wherein the assaying is directed to 100 or more genes or variants thereof selected from Table 1. In certain embodiments, the assaying covers at least 2,500 genes, gene fusions, point mutations, indels, copy-number variations, promoters, or enhancers. In certain embodiments, the first nucleic acid sample comprises cell-free DNA. In certain embodiments, 100 or more genes are assayed in the cell-free DNA. In certain embodiments, the first nucleic acid sample and the second nucleic acid sample are assayed for one or more genomic alterations at a concordance correlation coefficient of greater than or equal to about 90% when the first nucleic acid sample and the second nucleic acid sample are re-assayed for presence or absence of the genomic alterations, which genomic alterations include a plurality of different types of genomic alterations.

In certain aspects, the disclosure provides a method for analyzing a biological sample of a subject, comprising assaying the biological sample for a presence or absence of biological markers at a concordance correlation coefficient of greater than or equal to about 90% and an accuracy of at least about 90% as compared to a control when the biological sample is re-assayed for the presence or absence of the biological markers, which biological markers include a plurality of different types of biological markers, wherein the assaying comprises a plurality of different assays, including sequencing, wherein greater 90% of operations of the assaying are automatically performed.

In certain embodiments, the biological sample is homogenous. In certain embodiments, the biological sample comprises a tumor tissue or a whole blood sample from the subject. In certain embodiments, the biological sample comprises nucleic acid molecules. In certain embodiments, the biological sample comprises cell-free deoxyribonucleic acid (cfDNA) molecules, cellular deoxyribose nucleic acid (cDNA) molecules, ribonucleic acid (RNA) molecules, and protein, and wherein the cfDNA molecules, the cDNA molecules, and the RNA molecules are assayed for the presence or absence of the biological markers. In certain embodiments, the biological sample comprises normal biomolecules and abnormal biomolecules. In certain embodiments, the normal biomolecules are isolated from a buffy coat of the biological sample. In certain embodiments, the abnormal biomolecules are isolated from plasma or a tumor tissue of the biological sample. In certain embodiments, the biological sample is a single cell. In certain embodiments, biological sample is indexed. In certain embodiments, the method for analyzing a biological sample of a subject further comprises re-assaying the biological sample at a later point in time and identifying a change in one or more biological markers. In certain embodiments, the assaying comprises processing the biological sample or sequencing the biological sample without any involvement from a user during sample preparation. In certain embodiments, the assaying comprises immunohistochemistry profiling and genomic profiling of the biological sample. In certain embodiments, 2500 or greater of the biological markers are assayed. In certain embodiments, the assaying is at a concordance correlation coefficient of greater than or equal to about 90% and an accuracy of at least about 90% based on assaying the biological sample multiple times. In certain embodiments, the assaying is at a concordance correlation coefficient of greater than or equal to about 90% and an accuracy of at least about 90% based on assaying the biological sample in at least two different geographic locations.

In certain aspects, the disclosure provides a method for identifying a genomic aberration in one or more biological samples of a subject, comprising: (a) obtaining the one or more biological samples of the subject, which one or more biological samples comprise a nucleic acid sample that has or is suspected of having one or more genomic aberration(s) that appears at a frequency of less than about 5% in the nucleic acid sample; (b) enriching the nucleic acid sample for a plurality of nucleic acid sequences to provide an enriched nucleic acid sample using a probe set comprising probes that have an on-target rate as a group of at least about 80%, as determined by (i) measuring, for the probe set in at least one predetermined region, (1) probe coverage of each probe in the probe set and (2) off-target probe coverage for each probe in the probe set, and (ii) determining the on-target rate of the probe set based on a ratio of the off-target coverage to the probe coverage; (c) sequencing the enriched nucleic acid sample to generate sequencing reads; and (d) processing the sequencing reads to identify the genomic aberration(s) in the one or more biological samples of the subject that appears at a frequency of less than about 5% in the nucleic acid sample.

In certain embodiments, one or more biological samples comprise blood sample(s) or a tissue sample(s). In certain embodiments, the processing covers at least 2,500 genes, gene fusions, point mutations, indels, copy-number variations, promoters, or enhancers. In certain embodiments, the nucleic acid sample comprises cell-free DNA. In certain embodiments, one or more biological samples are indexed. In certain embodiments, the method for identifying a genomic aberration further comprises re-processing the biological sample at a later point in time and identifying a change in one or more biological markers. In certain embodiments, the processing comprises immunohistochemistry profiling and genomic profiling of the biological sample. In certain embodiments, 2500 or greater biological markers are assayed.

In certain aspects, the disclosure provides a system for providing a subject displaying cancer with a therapy, comprising: one or more computer memory comprising (i) biologic data of the subject, which biologic data is generated from one or more biological samples of the subject, or (ii) medical history data of the subject; and one or more computer processors operatively coupled to one or more databases of therapies, wherein the one or more computer processors are individually or collectively programmed to: (i) receive medical history data and biologic data for the subject, which biologic data is generated from one or more biological samples of the subject by automated handling from insertion into an automated system using at least one of the following steps of cell extraction, nucleic acid extraction, enrichment, sequencing, and immunohistochemistry, during processing of the one or more biological samples; (ii) analyze the medical history data and the biologic data to yield a genomic-based medical history analysis for the subject; (iii) use the genomic-based medical history analysis for the subject to query one or more databases of therapies for the subject, to generate a subset of therapies for which the subject qualifies; and (iv) electronically output the subset of therapies on a user interface for display to a user.

In certain embodiments, the one or more computer processors receive the biologic data or the medical history data over a network. In certain embodiments, the system for providing a subject displaying cancer with a therapy further comprises a sequencer that subjects the one or more biological samples to sequencing to generate the biologic data.

In certain aspects, the disclosure provides a non-transitory computer-readable medium comprising machine-executable code that, upon execution by one or more computer processors, implements a method for providing a subject displaying cancer with a therapy, comprising: (a) receiving medical history data and biologic data for the subject, which biologic data is generated from one or more biological samples of the subject by automated handling from insertion into an automated system using at least one of the following steps of cell extraction, nucleic acid extraction, enrichment, sequencing, and immunohistochemistry, during processing of the one or more biological samples; (b) analyzing the medical history data and the biologic data to yield a genomic-based medical history analysis for the subject; (c) using the genomic-based medical history analysis for the subject to query one or more databases of therapies for the subject, to generate a subset of therapies for which the subject qualifies; and (d) electronically outputting the subset of therapies on a user interface for display to a user.

In certain aspects, the disclosure provides a method for qualifying a subject for a subset of therapies, comprising: (a) subjecting at least one biological sample from the subject to at least one assay to generate biologic data from the subject; (b) processing the biologic data from the subject against a filtered set of therapies to generate the subset of therapies for which the subject qualifies, which filtered set of therapies is generated by computer assessing eligibility of a database of therapies against one or more criteria; (c) presenting the subset of therapies on a user interface on an electronic device of a user; and (d) further comprising transmitting medical history data of the subject to one or more therapy coordinators of the subset of therapies. In certain embodiments, the biologic data is generated from at least one biological sample of the subject by an automated assaying system, which automated assaying system uses automated processing for at least one member selected from the group consisting of cell extraction, nucleic acid extraction, enrichment, sequencing, and immunohistochemistry, during processing of the at least one biological sample.

In certain aspects, the disclosure provides a computer-implemented method for providing a subject displaying cancer with a therapy, comprising: (a) receiving biologic data for the subject, which biological data is generated from one or more biological samples of the subject; (b) using the biologic data to generate a first list of therapies according to a molecular profile of the subject, which molecular profile is indicative of one or more genomic aberrations in one or more biological samples; (c) generating a second list of therapies from the first list of therapies using medical history data of the subject; and (d) electronically outputting the second list of therapies. In certain embodiments, prior to (c), medical history data is received for the subject. In certain embodiments, prior to (c), the medical history data is processed and transformed to provide processed medical history data. In certain embodiments, the processing is selected from the group consisting of cleaning, organizing, and labeling. In certain embodiments, the processed medical history data is presented to the subject. In certain embodiments, the list of therapies comprises clinical trials and/or standard of care.

In certain embodiments, the computer-implemented method for providing a subject displaying cancer with a therapy further comprises presenting the second list of therapies on a user interface for display to the subject. In certain embodiments, the computer-implemented method for providing a subject displaying cancer with a therapy further comprises presenting the second list of therapies to a clinician to select for a recommended therapy. In certain embodiments, the computer-implemented method for providing a subject displaying cancer with a therapy further comprises receiving a request for enrollment of the subject in a given therapy selected from the second list of therapies.

In certain embodiments, the biologic data is generated from one or more biological samples of the subject without any pipetting by a user during preparation of one or more biological samples. In certain embodiments, the biologic data comprises data generated from one or more biological samples selected from the group consisting of protein, peptides, cell-free nucleic acids, ribonucleic acids, deoxyribose nucleic acids, and any combination thereof. In certain embodiments, one or more genomic aberrations include nucleic acid mutations and/or differentially expressed proteins. In certain embodiments, nucleic acid mutations are selected from the group consisting of an insertion(s), nucleotide deletion(s), nucleotide substitution(s), amino acid insertion(s), amino acid deletion(s), amino acid substitution(s), gene fusion(s), and copy-number variation(s). In certain embodiments, the nucleic acid mutations are selected from genes and variants of Table 1.

In certain embodiments, (b) of the computer-implemented method for providing a subject displaying cancer with a therapy comprises querying one or more databases for one or more targeted clinical trials and therapies according to a predetermined gene or genomic region. In certain embodiments, the first list of therapies in (b) excludes therapies that target genomic aberrations absent in one or more biological samples. In certain embodiments, (b) comprises removing therapies that target genomic aberrations absent in one or more biological samples. In certain embodiments, the first list of therapies in (b) is filtered according to clinical phases of the therapy.

In certain embodiments, the medical history data is identifiable according to relevant medical text segments. In certain embodiments, machine learning algorithms are further used to detect and label relevant medical text segments.

In certain embodiments, (c) of the computer-implemented method for providing a subject displaying cancer with a therapy comprises determining ineligible therapies according to a categorical score and rejecting ineligible therapies from remaining therapies to generate a filtered list of remaining therapies. In certain embodiments, the categorical score is selected from the group consisting of yes, maybe, and no. In certain embodiments, the filtered list of remaining therapies are compared and reviewed. The review may generate a second list of therapies. The second list of therapies may be passed to a user to manually verify eligibility using links to information from the medical history data and the biologic data for the subject. In certain embodiments, the user is a healthcare professional. In certain embodiments, the user is a primary care provider of the subject.

In certain embodiments, the computer-implemented method for providing a subject displaying cancer with a therapy further comprising filtering the second list of therapies based on filtering preferences of a user. The user may be the subject. In certain embodiments, the filtering preferences are selected from the group consisting of availability at a specific institution, availability at a set of institutions, type of treatment, phase of clinical trial, method of drug delivery, location and distance of a given therapy from a specified location, duration of treatment, and subject relocation therapy duration. In certain embodiments, the filtering further comprises an evaluation by a healthcare professional and a selection for a recommended therapy. In certain embodiments, the second list of therapies is generated from the first list of therapies without use of the molecular profile of the subject. In certain embodiments, the computer-implemented method for providing a subject displaying cancer with a therapy further comprises, prior to (a), subjecting one or more biological samples of the subject to sequencing to generate the biologic data.

In certain aspects, the disclosure provides a method for identifying a genomic aberration in one or more biological samples of a subject, comprising: (a) obtaining one or more biological samples of the subject, which one or more biological samples comprise a nucleic acid sample that has or is suspected of having one or more genomic aberration(s) that appears at a frequency of less than about 5% in the nucleic acid sample; (b) enriching the nucleic acid sample for a plurality of nucleic acid sequences to provide an enriched nucleic acid sample using a probe set comprising probes that have an on-target rate as a group of at least about 95%, as determined by (i) comparing the probe set to at least one predetermined region to measure (1) probe coverage of each probe in the probe set and (2) off-target probe coverage for each probe in the probe set, and (ii) determining the on-target rate of the probe set based on a ratio of the off-target coverage to the probe coverage; (c) sequencing the enriched nucleic acid sample to generate sequencing reads; and (d) processing the sequencing reads to identify one or more genomic aberration(s) in one or more biological samples of the subject that appears at a frequency of less than about 5% in the nucleic acid sample. In certain embodiments, one or more biological samples comprise blood sample(s) and/or a tissue sample(s). In certain embodiments, the tumor tissue sample is formalin-fixed, paraffin-embedded (FFPE) tissue. In certain embodiments, one or more biological samples is selected from the group consisting of protein, peptides, cell-free nucleic acids, ribonucleic acids, deoxyribose nucleic acids, and any combination thereof. In certain embodiments, one or more genomic aberrations include nucleic acid mutations. In certain embodiments, one or more genomic aberrations are selected from the group consisting of an insertion, nucleotide deletion, nucleotide substitution, amino acid insertion, amino acid deletion, amino acid substitution, gene fusion, copy-number variation, gene expression signatures, and any combination thereof.

In certain embodiments, the method for identifying a genomic aberration in one or more biological samples of a subject, further comprises using the probe set to generate a classifier for identifying the genomic aberration, which classifier is at least in part generated by: sequencing one or more predetermined regions of a genome from a tumor tissue sample of the subject to provide sequencing reads; in the sequencing reads, identifying sequences for the probe set that covers the one or more predetermined regions of a genome; comparing the probe set to one or more predetermined regions to measure (i) probe coverage of each probe in the probe set and (ii) off-target probe coverage for each probe in the probe set; determining an on-target rate of the probe set based on a ratio of the off-target coverage to the probe coverage; selecting a portion of the probe set that covers one or more predetermined regions of a genome and a portion of the probe set with an on-target rate of at least 95% in aggregate, thereby determining a custom probe set; and providing one or more features to permit classification of the probe set for one or more probes.

In certain embodiments, the classifier is used to identify a new set of probes, at least in part by: generating one or more features from the new set of probes; inputting one or more features from the new set of probes into the classifier; and using the classifier to predict a classification outcome for the new set of probes. In certain embodiments, one or more features is selected from the group consisting of sequence, sequence length, alignment location, probe coverage, off-target probe coverage, on target rate, genomic aberrations, genes, and variants of the genes. In certain embodiments, one or more features are selected from Table 1. In certain embodiments, the classification outcome is selected from a first outcome and a second outcome, wherein the first outcome directs a user to order the new set of probes and the second outcome does not direct the user to order the new set of probes.

In certain embodiments, the one or more predetermined region(s) comprise one or more components selected from the group consisting of one or more segments of a gene, one or more segments of a plurality of genes, coding sequences, non-coding sequences, at least 2600 genes, gene fusions, point mutations, indels, copy-number variations, promoters, and enhancers. In certain embodiments, the sequencing is selected from the group consisting of exome sequencing, transcriptome sequencing, genome sequencing, and cell-free DNA sequencing. In certain embodiments, the genome sequencing is targeted sequencing. In certain embodiments, the genome sequencing is untargeted sequencing.

In certain aspects, the disclosure provides a system for providing a subject displaying cancer with a therapy, comprising: one or more computer memory comprising (i) biologic data of the subject, which biologic data is generated from one or more biological samples of the subject, or (ii) medical history data of the subject; and one or more computer processors operatively coupled to the database, wherein one or more computer processors are individually or collectively programmed to: (i) receive biologic data of the subject from the database; (ii) use the biologic data to generate a first list of therapies according to a molecular profile of the subject, which molecular profile is indicative of one or more genomic aberrations in one or more biological samples; (iii) generate a second list of therapies from the first list of therapies using medical history data of the subject; and (iv) electronically output the second list of therapies.

In certain embodiments, one or more computer memory comprises biologic data of the subject and the medical history data of the subject. In certain embodiments, one or more computer processors receive the biologic data or the medical history data over a network. In certain embodiments, the system for providing a subject displaying cancer with a therapy further comprises a sequencer that subjects one or more biological samples to sequencing to generate the biologic data.

In certain aspects, the disclosure provides a non-transitory computer-readable medium comprising machine-executable code that, upon execution by one or more computer processors, implements a method for providing a subject displaying cancer with a therapy, comprising: (a) receiving biologic data for the subject, which biological data is generated from one or more biological samples of the subject; (b) using the biologic data to generate a first list of therapies according to a molecular profile of the subject, which molecular profile is indicative of one or more genomic aberrations in one or more biological samples; (c) generating a second list of therapies from the first list of therapies using medical history data of the subject; and (d) electronically outputting the second list of therapies.

In certain aspects, the disclosure provides a computer-implemented method for qualifying a subject for a clinical trial, comprising: (a) receiving medical history data and biologic data for the subject, which biologic data is generated from one or more biological samples of the subject without any pipetting by a user during preparation of the one or more biological samples; (b) querying one or more databases for one or more clinical trials corresponding to the medical history data and the biologic data for the subject to generate a set of clinical trials for which the subject qualifies, which set of clinical trials comprises at least one clinical trial; (c) providing the set of clinical trials on a user interface for display to a user; and (d) receiving a request for enrollment of the subject in a clinical trial selected from the provided set of clinical trials through the user interface.

In certain embodiments, (a) comprises receiving phenotype information for the subject. In certain embodiments, the phenotype information comprises one or more of age, weight, height, sex, race, body mass index (BMI), previous treatments and response, eastern cooperative oncology group (ECOG) score, and diagnosis. In certain embodiments, computer-implemented method for qualifying a subject further comprises automatically generating the biologic data from the one or more biological samples of the subject without any involvement of the user. In certain embodiments, computer-implemented method for qualifying a subject further comprises prioritizing the one or more clinical trials within the generated set of clinical trials. In certain embodiments, prioritizing is based on one or more factors selected from the group consisting of: geographic location of the clinical trial, regulatory approval status, annotated medical history data for the subject, or a combination thereof. In certain embodiments, computer-implemented method for qualifying a subject further comprises enrolling the subject in the clinical trial. In certain embodiments, computer-implemented method for qualifying a subject further comprises (e) monitoring the subject enrolled in the clinical trial by assaying the one or more biological samples from the subject, wherein assaying is directed to 100 or more genes or variants thereof selected from Table 1. In certain embodiments, computer-implemented method for qualifying a subject further comprises predicting a likelihood of success for the subject. In certain embodiments, the one or more clinical trials are annotated. In certain embodiments, the querying of (b) has a predicted likelihood of matching to a clinical trial of at least about 90%. In certain embodiments, the request is received over a network. In certain embodiments, the one or more biological samples comprise a blood sample. In certain embodiments, one or more biological samples comprise a tumor tissue sample and a normal tissue sample. In certain embodiments, the tumor tissue sample is a formalin-fixed paraffin embedded (FFPE) tissue sample. In certain embodiments, the receiving of (a) comprises receiving (i) a first biological sample from the tumor tissue sample of the subject, and (ii) a second biological sample from the normal tissue sample of the subject, and assaying the first biological sample and the second biological sample to identify the one or more biological markers in the tumor tissue sample relative to the normal tissue sample to generate a set of biologic data for the subject. In certain embodiments, one or more biological samples are assayed for a presence or absence of biological markers at a concordance correlation coefficient of greater than or equal to about 90% when the biological sample is re-assayed for the presence or absence of the biological markers, which biological markers include a plurality of different types of biological markers. In certain embodiments, the plurality of different types of biological markers are selected from the group consisting of one or more nucleotide insertions, nucleotide deletions, nucleotide substitutions, amino acid insertions, amino acid deletions, amino acid substitutions, gene fusions, copy-number variations, and any combination thereof. In certain embodiments, assaying is directed to two or more genes or variants thereof selected from Table 1. In certain embodiments, assaying is directed to 100 or more genes or variants thereof selected from Table 1. In certain embodiments, the assaying covers at least 2,500 genes, gene fusions, point mutations, indels, copy-number variations, promoters, and/or enhancers. In certain embodiments, biologic data comprises one or more genomic alterations are selected from the group consisting of one or more nucleotide insertions, nucleotide deletions, nucleotide substitutions, amino acid insertions, amino acid deletions, amino acid substitutions, gene fusions, copy-number variations, and any combination thereof. In certain embodiments, the biologic data comprises data from one or more biological sample components selected from the group consisting of: protein, peptides, cell-free nucleic acids, ribonucleic acids, deoxyribose nucleic acids, and any combination thereof.

In certain embodiments, the subject is diagnosed with a solid tumor or cancer. In certain embodiments, the medical history data is automatically annotated. In certain embodiments, the medical history data is annotated in standardized terminology. In certain embodiments, the standardized terminology is Unified Medical Language System. In certain embodiments, the user interface is a web-based user interface or mobile user interface. In certain embodiments, the biologic data is automatically generated from one or more biological samples of the subject without any involvement of the user during the preparation.

In certain aspects, the disclosure provides a method for qualifying a subject for a clinical trial, comprising: (a) receiving (i) a first nucleic acid sample from a tumor tissue sample of the subject, and (ii) a second nucleic acid sample from a normal tissue sample of the subject; (b) assaying the first nucleic acid sample and the second nucleic acid sample to identify the one or more genomic alterations in the tumor tissue sample relative to the normal tissue sample to generate a set of genomic data for the subject, wherein the assaying is performed without any pipetting by a user during preparation of the first nucleic acid sample and the second nucleic acid sample prior to identifying the one or more genomic alternations; (c) querying one or more databases for one or more clinical trials corresponding to a medical history of the subject and the genomic data to generate a set of clinical trials for which the subject qualifies; and providing the set of clinical trials on a user interface for display to a user.

In certain embodiments, the method for qualifying a subject further comprises receiving medical history data for the subject. In certain embodiments, the method for qualifying a subject further comprises (e) receiving a request for enrollment of the subject in a clinical trial selected from the provided set of clinical trials through the user interface. In certain embodiments, the method for qualifying a subject further comprises identifying a therapeutic target based on the medical history and the genomic data and enrolling the subject in a clinical trial based on the identified target. In certain embodiments, the method for qualifying a subject further comprises monitoring the subject, the monitoring comprising assaying one or more nucleic acid samples to generate genomic data, wherein the assaying is directed to 100 or more genes or variants thereof selected from Table 1. In certain embodiments, the normal tissue sample comprises blood. In certain embodiments, the tumor tissue sample is formalin-fixed, paraffin-embedded (FFPE) tissue.

In certain embodiments, assaying is directed to two or more genes or variants thereof selected from Table 1. In certain embodiments, assaying is directed to 100 or more genes or variants thereof selected from Table 1. In certain embodiments, assaying covers at least 2,500 genes, gene fusions, point mutations, indels, copy-number variations, promoters, and/or enhancers. In certain embodiments, the first nucleic acid sample comprises cell-free DNA. In certain embodiments, 100 or more genes are assayed in the cell-free DNA. In certain embodiments, assaying comprises sequencing the first nucleic acid sample and the second nucleic acid sample. In certain embodiments, sequencing is performed without any involvement from the user. In certain embodiments, assaying further comprises receiving a request from the user to sequence the biological sample. In certain embodiments, the sequencing is selected from the group consisting of exome sequencing, transcriptome sequencing, genome sequencing, and cell-free DNA sequencing. In certain embodiments, the first nucleic acid sample and second nucleic acid sample are assayed for one or more genomic alterations at a concordance correlation coefficient of greater than or equal to about 90% when the first nucleic acid sample and second nucleic acid sample are re-assayed for the presence or absence of the genomic alterations, which genomic alterations include a plurality of different types of genomic alterations. In certain embodiments, the types of genomic alteration are selected from the group consisting of: nucleotide insertions, nucleotide deletions, nucleotide substitutions, gene fusions, and copy-number variations. In certain embodiments, the method for qualifying a subject further comprises receiving a request from the user to sequence the first nucleic acid sample and the second nucleic acid sample. In certain embodiments, assaying comprises subjecting the first nucleic acid sample and the second nucleic acid sample to sequencing to detect at least 5 genes or variants thereof selected from Table 1. In certain embodiments, the assaying comprises subjecting the first nucleic acid sample and the second nucleic acid sample to sequencing to detect at least 10 genes or variants thereof selected from Table 1. In certain embodiments, assaying comprises subjecting the first nucleic acid sample and the second nucleic acid sample to sequencing to detect at least 15 genes or variants thereof selected from Table 1. In certain embodiments, the assaying comprises subjecting the first nucleic acid sample and the second nucleic acid sample to sequencing to detect at least 20 genes or variants thereof selected from Table 1. In certain embodiments, the assaying comprises subjecting the first nucleic acid sample and the second nucleic acid sample to sequencing to detect at least 30 genes or variants thereof selected from Table 1. In certain embodiments, the assaying comprises subjecting the first nucleic acid sample and the second nucleic acid sample to sequencing to detect at least 40 genes or variants thereof selected from Table 1. In certain embodiments, the first nucleic acid sample and second nucleic acid sample are obtained from the tumor tissue sample and the normal tissue sample without any pipetting by the user. In certain embodiments, the first nucleic acid sample and second nucleic acid sample are obtained from the tumor tissue sample and the normal tissue sample automatically without any involvement from the user.

In certain aspects, the disclosure provides a method for analyzing a biological sample of a subject, comprising assaying the biological sample for a presence or absence of biological markers at a concordance correlation coefficient of greater than or equal to about 90% and an accuracy of at least about 90% as compared to a control, when the biological sample is re-assayed for the presence or absence of the biological markers, which biological markers include a plurality of different types of biological markers, wherein the assaying comprises a plurality of different assays, including sequencing.

In certain embodiments, the biological sample is a tumor tissue sample. In certain embodiments, the biological sample is homogenous. In certain embodiments, the biological sample is a blood sample comprising plasma and a buffy coat. In certain embodiments, the biological sample comprises tumor tissue and whole blood from the subject. In certain embodiments, the biological sample comprises nucleic acid molecules. In certain embodiments, the biological sample comprises cell-free deoxyribonucleic acid (cfDNA) molecules, cellular deoxyribose nucleic acid (cDNA) molecules, ribonucleic acid (RNA) molecules, and protein, and wherein the cfDNA molecules, the cDNA molecules, and the RNA molecules are assayed for the presence or absence of the biological markers. In certain embodiments, the biological sample comprises normal biomolecules and abnormal biomolecules. In certain embodiments, the normal biomolecules are isolated from a buffy coat of the biological sample. In certain embodiments, the abnormal biomolecules are isolated from plasma or a tumor tissue of the biological sample. In certain embodiments, assaying the biological sample comprises comparing the normal biomolecules to the abnormal biomolecules.

In certain embodiments, the biological sample is a single cell. In certain embodiments, the biological sample is indexed. In certain embodiments, the method for analyzing a biological sample of a subject further comprises re-assaying the biological sample at a later point in time and identifying a change in one or more biological markers. In certain embodiments, assaying comprises processing the biological sample or sequencing the biological sample without any involvement from a user during sample preparation. In certain embodiments, sequencing is selected from the group consisting of exome sequencing, transcriptome sequencing, genome sequencing, and cell-free DNA sequencing. In certain embodiments, assaying begins after a user inputs the biological sample. In certain embodiments, assaying comprises immunohistochemistry profiling and genomic profiling of the biological sample. In certain embodiments, the method for analyzing a biological sample of a subject further comprises receiving a request from the user to process the biological sample or sequence the biological sample. In certain embodiments, the plurality of different types of biological markers are selected from the group consisting of one or more nucleotide insertions, nucleotide deletions, nucleotide substitutions, amino acid insertions, amino acid deletions, amino acid substitutions, gene fusions, copy-number variations, and any combination thereof. In certain embodiments, 2500 or greater biological markers are assayed. In certain embodiments, assaying comprises assaying 100 or greater biological markers in cell-free DNA of the biological sample. In certain embodiments, the plurality of different types of biological markers comprises antigens and genetic alterations. In certain embodiments, the plurality of different types of biological markers comprises antigens and genetic alterations. In certain embodiments, the method for analyzing a biological sample of a subject further comprises selecting a clinical trial based on the presence or absence of biological markers. In certain embodiments, the control is a healthy control. In certain embodiments, the control is from the subject. In certain embodiments, the assaying includes performing an assay that is not sequencing. In certain embodiments, the assaying is at a concordance correlation coefficient of greater than or equal to about 90% and an accuracy of at least about 90% based on assaying the biological sample multiple times. In certain embodiments, the assaying is at a concordance correlation coefficient of greater than or equal to about 90% and an accuracy of at least about 90% based on assaying the biological sample in at least two different geographic locations. In certain embodiments, the concordance correlation coefficient is greater than or equal to about 95%. In certain embodiments, the concordance correlation coefficient is greater than or equal to about 99%. In certain embodiments, the assaying comprises retrieving the biological sample and processing the biological sample, which processing is in the absence of pipetting.

In certain aspects, the disclosure provides a method for identifying one or more somatic mutations in a subject, comprising: (a) obtaining a tumor biological sample and normal biological sample from the subject; (b) assaying the tumor biological sample and the normal biological sample to (i) obtain sequence information for a first nucleic acid sample and a second nucleic acid sample obtained from the tumor biological sample and the normal biological sample, respectively, without any pipetting by a user during preparation of the first nucleic acid sample and the second nucleic acid sample prior to sequencing, and (ii) identify one or more other biological markers of a type different than the first nucleic acid sample and the second nucleic acid sample; (c) comparing the sequence information obtained for the first nucleic acid sample and the second nucleic acid sample to identify one or more genomic alterations in the tumor biological sample relative to the normal biological sample; and (d) using the (i) one or more other biological markers identified in (b) and (ii) the one or more genomic alterations identified in (c) to identify the one or more somatic mutations in the subject at an accuracy of at least about 90% as compared to a control.

In certain embodiments, the first nucleic acid sample and the second nucleic acid sample are automatically obtained from the tumor biological sample and the normal biological sample, respectively. In certain embodiments, the first nucleic acid sample and the second nucleic acid sample are automatically obtained from the tumor biological sample and the normal biological sample, respectively, without any involvement of the user during the preparation. In certain embodiments, the method for identifying one or more somatic mutations further comprises prior to (b), automatically obtaining (i) the first nucleic acid sample from the tumor biological sample of the subject and (ii) the second nucleic acid sample from the normal biological sample of the subject, without any involvement from the user. In certain embodiments, the tumor biological sample and the normal biological sample are obtained from a sample of blood comprising plasma and buffy coat from the subject. In certain embodiments, the first nucleic acid sample is obtained from cell-free DNA in the plasma. In certain embodiments, the tumor biological sample is a formalin-fixed paraffin embedded (FFPE) tissue sample. In certain embodiments, the normal biological sample is a buffy coat sample. In certain embodiments, the sequencing is selected from the group consisting of exome sequencing, transcriptome sequencing, genome sequencing, and cell-free DNA sequencing. In certain embodiments, the cell-free DNA sequencing comprises mismatch targeted sequencing (Mita-Seq) or tethered elimination of termini (Tet-Seq). In certain embodiments, the method for identifying one or more somatic mutations further comprises receiving a request from the user to sequence the first nucleic acid sample and the second nucleic acid sample. In certain embodiments, the sequencing covers at least 2,500 genes, gene fusions, point mutations, indels, copy-number variations, promoters, and/or enhancers. In certain embodiments, the sequencing is directed to two or more genes or variants thereof selected from Table 1. In certain embodiments, the sequencing is directed to 100 or more genes or variants thereof selected from Table 1. In certain embodiments, the one or more genomic alterations are selected from the group consisting of one or more nucleotide insertions, nucleotide deletions, nucleotide substitutions, amino acid insertions, amino acid deletions, amino acid substitutions, gene fusions, copy-number variations, and any combination thereof.

In certain embodiments, the subject is diagnosed with a solid tumor or cancer. In certain embodiments, the method for identifying one or more somatic mutations further comprises indexing the first nucleic acid sample and the second nucleic acid sample. In certain embodiments, the first nucleic acid sample and the second nucleic acid sample are assayed for one or more genomic alterations at a concordance correlation coefficient of greater than or equal to about 90% when the first nucleic acid sample and the second nucleic acid sample are re-assayed for the presence or absence of the genomic alterations, which genomic alterations include a plurality of different types of genomic alterations. In certain embodiments, the types of genomic alterations are selected from the group consisting of: nucleotide insertions, nucleotide deletions, nucleotide substitutions, gene fusions, and copy-number variations. In certain embodiments, the one or more genomic alterations are identified at an accuracy of at least about 90%.

Another aspect of the present disclosure provides a non-transitory computer readable medium comprising machine executable code that, upon execution by one or more computer processors, implements any of the methods above or elsewhere herein.

Another aspect of the present disclosure provides a computer system comprising one or more computer processors and a non-transitory computer readable medium coupled thereto. The non-transitory computer readable medium comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a workflow of the present disclosure;

FIG. 2 shows the biological sample processing workflow system;

FIG. 3a shows the platform situated in a laboratory setting;

FIG. 3b shows the system layout from above the wall of the laboratory between the two subunits;

FIGS. 4a-4c show several views and various components of a pre-amplification system;

FIGS. 5a-5c show several views and various components of a post-amplification system;

FIG. 6 shows the schematic of the platform for analysis of medical history and biological samples;

FIG. 7 shows the schematic for processing of a subject's medical records;

FIG. 8 shows an example profile of a subject after the completion of treatment matching;

FIG. 9 shows a route for qualifying a subject for enrollment in a clinical trial;

FIG. 10 shows another route for qualifying a subject for enrollment in a clinical trial;

FIG. 11 shows a clinical trial curation process according to eligibility defined by labels;

FIG. 12 shows another route for qualifying a subject for enrollment in a clinical trial using medical history and biologic data labels;

FIG. 13 shows a computer control system that is programmed or otherwise configured to implement methods provided herein; and

FIG. 14 shows an overview of the bioinformatics pipeline.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “genetic variant,” as used herein, generally refers to an alteration, variant or polymorphism in a nucleic acid sample or genome of a subject. Such alteration, variant or polymorphism can be with respect to a reference genome, which may be a reference genome of the subject or other individual. Single nucleotide polymorphisms (SNPs) are a form of polymorphisms. In some examples, one or more polymorphisms comprise one or more single nucleotide variations (SNVs), insertions, deletions, repeats, small insertions, small deletions, small repeats, structural variant junctions, variable length tandem repeats, and/or flanking sequences. Copy number variants (CNVs) and other rearrangements are also forms of genetic variation. A genomic alternation may be or include a base change, insertion, deletion, repeat, copy number variation, or structural rearrangement.

The term “polynucleotide,” as used herein, generally refers to a molecule comprising one or more nucleic acid subunits. A polynucleotide can include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a polynucleotide is deoxyribonucleic acid (DNA) or ribonucleic acid (RNA), or derivatives thereof. A polynucleotide can be single-stranded or double stranded.

The term “subject,” as used herein, generally refers to an animal, such as a mammalian species (e.g., human) or avian (e.g., bird) species, or other organism, such as a plant. More specifically, the subject can be a vertebrate, a mammal, a mouse, a primate, a simian or a human. Animals include, but are not limited to, farm animals, sport animals, and pets. A subject can be a healthy individual, an individual that has or is suspected of having a disease or a pre-disposition to the disease, or an individual that is in need of therapy or suspected of needing therapy. A subject can be a patient.

The term “sample,” as used herein, generally refers can be any biological sample isolated from a subject. For example, a sample can comprise, without limitation, bodily fluid, whole blood, platelets, serum, plasma, stool, red blood cells, white blood cells or leucocytes, endothelial cells, tissue biopsies, synovial fluid, lymphatic fluid, ascites fluid, interstitial or extracellular fluid, the fluid in spaces between cells, including gingival crevicular fluid, bone marrow, cerebrospinal fluid, plueral fluid, saliva, mucous, sputum, semen, sweat, urine, or any other bodily fluids. A bodily fluid can include saliva, blood, or serum. For example, a polynucleotide can be cell-free DNA and/or cell-free RNA (e.g., transcripts) isolated from a bodily fluid, e.g., blood or serum. A sample can also be a tumor sample, which can be obtained from a subject by various approaches, including, but not limited to, venipuncture, excretion, ejaculation, massage, biopsy, needle aspirate, lavage, scraping, surgical incision, or intervention or other approaches.

The term “genome” generally refers to an entirety of an organism's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together constitutes a human genome.

As used herein, the term “sequencing” is used in a broad sense and may refer to any technique that allows the order of at least some consecutive nucleotides in at least part of a nucleic acid to be identified, including without limitation at least part of an extension product or a vector insert.

The terms “adaptor(s)”, “adapter(s)” and “tag(s)” are used synonymously throughout this specification. An adaptor or tag can be coupled to a polynucleotide sequence to be “tagged” by any approach including ligation, hybridization, or other approaches. Adaptors may be unidirectional or bidirectional. Adaptors may be blunt-ended or have overhang ends.

The term “sequencing adaptor,” as used herein, generally refers to a molecule (e.g., polynucleotide) that is adapted to permit a sequencing instrument to sequence a target polynucleotide, such as by interacting with the target polynucleotide to enable sequencing. The sequencing adaptor permits the target polynucleotide to be sequenced by the sequencing instrument. In an example, the sequencing adaptor comprises a nucleotide sequence that hybridizes or binds to a capture polynucleotide attached to a solid support of a sequencing system, such as a flow cell. In another example, the sequencing adaptor comprises a nucleotide sequence that hybridizes or binds to a polynucleotide to generate a hairpin loop, which permits the target polynucleotide to be sequenced by a sequencing system. The sequencing adaptor can include a sequencer motif, which can be a nucleotide sequence that is complementary to a flow cell sequence of other molecule (e.g., polynucleotide) and usable by the sequencing system to sequence the target polynucleotide. The sequencer motif can also include a primer sequence for use in sequencing, such as sequencing by synthesis. The sequencer motif can include the sequence(s) needed to couple a library adaptor to a sequencing system and sequence the target polynucleotide.

As used herein the terms “at least”, “at most” or “about”, when preceding a series, refers to each member of the series, unless otherwise identified.

The term “about” and its grammatical equivalents in relation to a reference numerical value can include a range of values up to plus or minus 10% from that value. For example, the amount “about 10” can include amounts from 9 to 11. In other embodiments, the term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

The term “label,” as used herein, generally refers to one or more strings of characters. A label may be text string, a numerical string, alphanumerical string, or a string of characters. A label may identify a relevant portion of certain biological data, medical history data, or clinical trial data.

The present disclosure provides methods for analyzing a biological sample of a subject and for clinical diagnosis and testing, such as screening (for example for breast cancer as is common in women over 50), scans, such as magnetic resonance imaging (MRI) scans, computerized tomography (CT) scans, or body fluid testing (for instance blood tests).

A subject with a genetic susceptibility may be diagnosed with a specific condition. Such conditions can include cancer, a solid tumor, obesity, autoimmune diseases, heart disease, AIDS at the onset of which is known to occur at different times in otherwise similar individuals, blood pressure control, asthma, diabetes and other chronic diseases. Autoimmune diseases may include hay fever and arthritis. Depression may include conditions such as Major Depression, Dysthymic Disorder, Unspecified Depression, Adjustment Disorder (with Depression) and Bipolar Depression.

The subject may also be diagnosed with cancer, such as acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, bowl cancer, cancers of the blood, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, Wilms Tumor and/or other tumors.

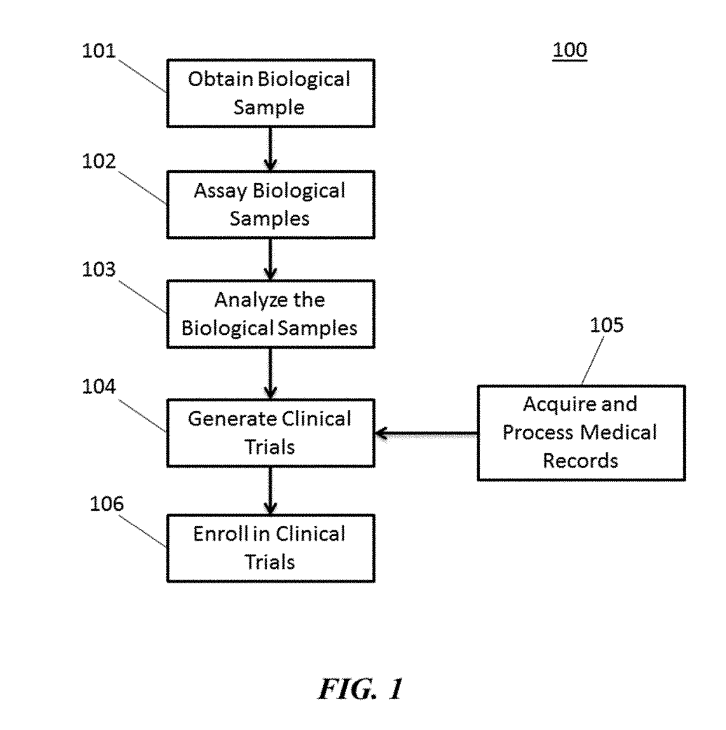

FIG. 1 shows a workflow 100. In a first operation, one or more biological samples of a subject 101 (e.g., a tumor and normal sample) may be obtained. The one or more biological samples may be subjected to assaying to identify a disease in a subject 102. Next, the biological sample may be analyzed 103 using a computer implemented method to extract data from the one or more biological samples for clinical trial enrollment and drug development. Clinical trials may then be generated 104 from the data. Medical records may then be acquired and processed to extract relevant clinical information 105. The subject may then be enrolled into a clinical trial(s) 106. Such enrollment may be automatic or upon request by the subject or another user (e.g., healthcare provider of the subject). The subject may be a patient.

The workflow 100 is capable of generating clinical trial matches and/or standard of care treatment options. Under operation 105, a subject's medical records may be acquired and processed to extract relevant clinical information.

### Analysis of Biological Samples

In an aspect, the present disclosure provides a method for analyzing a biological sample of a subject, comprising assaying a biological sample for a presence or absence of biological markers at a concordance correlation coefficient of greater than or equal to about 90% and an accuracy of at least about 90% as compared to a control. The concordance correlation coefficient may be greater than or equal to about 60%, 70%, 80%, 90%, 95%, 96%, 97%, 98%, or 99%. The accuracy may be at least about 60%, about 70%, about 80%, or about 90%. The accuracy may be at least about 60%, 70%, 80%, 90%, 95%, 96%, 97%, 98%, or 99%. The biological sample may be re-assayed for the presence or absence of the biological markers. The biological sample may be homogenous. The biological markers may include a plurality of different types of biological markers. At least about 500 biological markers, 1000 biological markers, 1500 biological markers, 2000 biological markers, 2500 biological markers, 3000 biological markers, 3500 biological markers, or 4000 biological markers can be assayed.

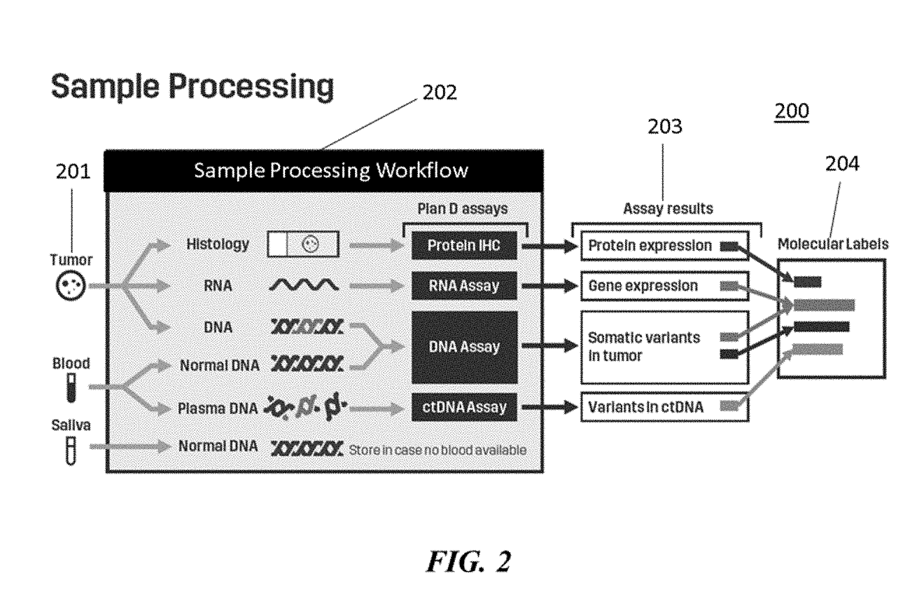

FIG. 2 shows the biological sample processing workflow system 200. The biological sample 201 may be a tumor sample, a blood sample, or a saliva sample. During the biological sample processing 202, protein, DNA, and RNA may be extracted from the tumor sample and may undergo protein immunohistochemistry (IHC), RNA assay, and DNA assay described herein. Normal DNA and plasma DNA may be extracted from the blood sample and may undergo DNA assay and circulating tumor DNA (ctDNA) assay respectively as described herein. Normal DNA may be extracted from the saliva sample and stored as a back up sample supply in the absence of blood samples. Following biological sample processing, the results of gene expression, protein expression, somatic variants in tumor, and variants in ctDNA are reported 203 and labeled according to the labels to generate the labeled biologics data 204.

Biological samples may include fluid and/or tissue from a subject. The biological sample may be a tumor biological sample or a normal biological sample. A control may be obtained from the subject. The control may be a healthy control or normal biological sample. The biological sample to be tested may be whole blood, or saliva. The biological sample can comprise plasma, a buffy coat, or saliva. A buffy coat may comprise lymphocytes, thrombocytes, and leukocytes. A tumor sample may include a tumor tissue biopsy and/or circulating tumor DNA in a cell-free DNA sample. The normal sample can include buffy coat cells, whole blood, or normal epithelial cells. Buffy coat cells may be white blood cells. The normal sample can include nucleic acid molecules derived from the white blood cells or epithelial cells in the saliva. Normal DNA may be extracted from the white blood cells or epithelial cells in the saliva. A sample can comprise nucleic acids from different sources. For example, a sample can comprise germline DNA or somatic DNA. A sample can comprise nucleic acids carrying mutations. For example, a sample can comprise DNA carrying germline mutations and/or somatic mutations. A sample can also comprise DNA carrying cancer-associated mutations (e.g., cancer-associated somatic mutations). Tumor and normal cells may be compared. The tumor sample may be compared to the various normal samples. A sample can comprise RNA (e.g., mRNA), which may be sequenced (e.g., via reverse transcription of RNA and subsequent sequencing of cDNA).

A biological fluid can include any untreated or treated fluid associated with living organisms. Examples can include, but are not limited to, blood, including whole blood, warm or cold blood, and stored or fresh blood; treated blood, such as blood diluted with at least one physiological solution, including but not limited to saline, nutrient and/or anticoagulant solutions; blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC); analogous blood products derived from blood or a blood component or derived from bone marrow; red cells separated from plasma and resuspended in physiological fluid or a cryoprotective fluid; and platelets separated from plasma and resuspended in physiological fluid or a cryoprotective fluid. Other non-limiting examples of biological samples include skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, skin cells, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, micropiota, meconium, breast milk, and/or other excretions or body tissues. Results from blood samples may be obtained after at least about 1 minute, 5 minutes, 10 minutes, 20 minutes, 30 minutes, 1 hour, 2 hours, 3 hours, 4 hours, 5 hours, 6 hours, 12 hours, 1 day, 2 days, 3 days, 4 days, 5 days, 6 days, 7 days, 8 days, 9 days, 10 days, or longer.

A sample can also be a tumor sample, which can be obtained from a subject by various approaches, including, but not limited to, venipuncture, excretion, ejaculation, massage, biopsy, needle aspirate, lavage, scraping, surgical incision, or intervention or other approaches. The tumor sample may be a tumor tissue sample.

The biological sample can comprise nucleic acid molecules from different sources. For example, a sample can comprise germline DNA or somatic DNA. A sample can comprise nucleic acids carrying mutations. For example, a sample can comprise DNA carrying germline mutations and/or somatic mutations. A sample can also comprise DNA carrying cancer-associated mutations (e.g., cancer-associated somatic mutations).

A sample can comprise various amount of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cell-free DNA (cfDNA), about 600 billion individual molecules.

The biological sample may be a tissue sample. A tissue may be a group of connected specialized cells that perform a special function. The tissue may also be an extracellular matrix material. The tissue analyzed can be a portion of a tissue to be transplanted or surgically grafted, such as an organ (e.g., heart, kidney, liver, lung, etc.), skin, bone, nervous tissue, tendons, blood vessels, fat, cornea, blood, or a blood component.

Examples of tissue may be selected from a group consisting of placental tissue, mammary gland tissue, gastrointestinal tissue, liver tissue, kidney tissue, musculoskeletal tissue, genitourinary tissue, bone marrow tissue, prostate tissue, skin tissue, nasal passage tissue, neural tissue, eye tissue, and central nervous system tissue. The tissue may originate from a human and or mammal. The tissue can comprise the connecting material and the liquid material found in association with the cells and/or tissues. A tissue can also include biopsied tissue and media containing cells or biological material. The biological sample may be a tumor tissue sample.

Tissue from a subject may be preserved for research that involves maintaining molecule and morphological integrity. The preservation methods of tissue for latter downstream usage can include freezing media embedded tissue, flash freezing tissue, and formalin-fixed paraffin embedded (FFPE tissue). The preservation method may also include blood sample collection, transport, and storage in a direct draw whole blood collection tube. The collection tube may be a Cell-Free DNA BCT®. The Cell-Free DNA BCT can stabilize cell-free plasma DNA and can preserve cellular genomic DNA found in nucleated blood cells and circulating epithelial cells in whole blood. Blood may be preserved in blood collection tubes.

The tumor biological sample may be a formalin-fixed paraffin embedded (FFPE) tissue sample. Paraformaldehyde may be used for tissue fixation. The tissue can be sliced or used as a whole. Prior to sectioning, the tissue can be embedded in cryomedia or paraffin wax. A microtome or a cryostat may be used to section the tissue. The sections may be mounted onto slides, dehydrated with alcohol washes and cleared with a detergent. The detergent may be xylene or citrisolv. For FFPE tissues, antigen retrieval may occur by thermal pre-treatment or protease pre-treatment of the sections.

Cells and other biocomponents in a biological sample may be analyzed using antibodies (e.g., immunohistochemistry, western blot, enzyme linked immunosorbent assay (ELISA), mass spectrometry, antibody staining, radioimmunoassay, fluoroimmunoassay, chemiluminescence immunoassay, and liposome immunoassay). Primary cells may be isolated from small fragments of tissue and purified from the blood. The primary cells may include lymphocytes (white blood cells), fibroblasts (skin biopsy cells), or epithelial cells. The biological sample may be a single cell. Before antibody staining, endogenous biotin or enzymes can be quenched. Biological samples may be incubated with buffer for blockage of reactive sites in which primary or secondary antibodies can bind. This step may help with reducing non-specific binding between the antibodies and non-specific proteins resulting in background staining. Blocking buffers may be selected from the group consisting of non-fat dry milk, normal serum, gelatin, or bovine serum albumin. Background staining may be reduced by methods selected from the group consisting of dilution of the primary or secondary antibodies, use of different detection system or a different primary antibody, and changing the time or temperature of the incubation. Tissue known to express the antigen and tissue not known to express the antigen may be used as a control.

The biological sample obtainable from specimens or fluids can include detached tumor cells or free nucleic acids that are released from dead or damaged tumor cells. Nucleic acids may include deoxyribonucleic acid (DNA), cell free-deoxyribonucleic acid (cfDNA) molecules, cellular deoxyribose nucleic acid (cDNA) molecules, ribonucleic acid (RNA) molecules, genomic DNA molecules, mitochondrial DNA molecules, single or double stranded DNA molecules, and protein-associated nucleic acids. Any nucleic acid specimen in purified or non-purified form obtained from such specimen cell can be utilized as the starting nucleic acid or acids. The cfDNA molecules, cDNA molecules, and RNA molecules may be assayed for presence or absence of biological markers.

Biological data may be obtained from the biological samples. Biologic data may comprise data from one or more biological sample components selected from the group consisting of: protein, peptides, cell-free nucleic acids, ribonucleic acids, deoxyribose nucleic acids, and any combination thereof.

The biomolecules may be normal and abnormal. The normal biomolecules may be isolated from the buffy coat of the biological sample. The abnormal biomolecules may be isolated from the plasma or a tumor tissue of the biological sample. A sample can comprise nucleic acids from different sources. For example, a sample can comprise germline DNA or somatic DNA. A sample can comprise nucleic acids carrying mutations. For example, a sample can comprise DNA carrying germline mutations and/or somatic mutations. A sample can also comprise DNA carrying cancer-associated mutations (e.g., cancer-associated somatic mutations).

A biological sample of components may be analyzed with respect to various biomarkers. Biomarkers can be indicators of or a proxy for various biological phenomena. The presence or absence of a biological marker, a quantity or quality thereof can be indicative of a biological process of phenomena. Biomarkers (biological markers) may be a characteristic that is objectively measured and determined as an indicator of normal biological processes, pathogenic processes, pharmacologic responses to a therapeutic intervention, or environmental exposure. Biomarkers may be categorized into DNA biomarkers, DNA tumor biomarkers, and general biomarkers. Biomarkers can be selected from the group consisting of cancer biomarker, clinical endpoint, companion endpoint, copy number variant (CNV) biomarker, diagnostic biomarker, disease biomarker, DNA biomarker efficacy biomarker, epigenetic biomarker, monitoring biomarker, prognostic biomarker, predictive biomarker, safety biomarker, screening biomarker, staging biomarker, stratification biomarker, surrogate biomarker, target biomarker, target biomarker, and toxicity biomarker. Diagnostic biomarkers may be used to diagnose a disease or decide on the severity of a disease. DNA biomarkers can comprise interleukin 28B (IL28B) or solute carrier organic anion transporter family member 1B1 (SLCO1B1). DNA tumor biomarkers may comprise BluePrint®, epidermal growth factor receptor (EGFR), Kirsten rat scarcoma viral oncogene homologue (K-Ras), MammaPrint®, and OncoTypDX®. General biomarkers may be a point of care test, such as RheumaChec or CCPoint assay.

### Methods of Obtaining Biological Samples and Biomolecules

The biological sample may comprise normal biomolecules and abnormal biomolecules extracted from a subject. DNA extraction may be obtained from buccal swabs, hair sample, urine sample, blood sample, and a tissue sample. During a biopsy, sample of cells and tissue may be removed from the subject's body for analysis in a laboratory. Biopsy may be selected from the group consisting of advanced breast biopsy instrumentation, brush biopsy, computed tomography, cone biopsy, core biopsy, Crosby capsule, curettings, ductal lavage, endoscopic biopsy, endoscopic retrograde cholangiopancreatography, evacuation, excision biopsy, fine needle aspiration, fluoroscopy, frozen section, imprint, incision biopsy, liquid based cytology, loop electrosurgical excision procedure, magnetic resonance imaging, mammography, needle biopsy, positron emission tomography with fluorodeoxy-glucose, punch biopsy, sentinel node biopsy, shave biopsy, smears, stereotactic biopsy, transurethral resection, trephine (bone marrow) biopsy, ultrasound, vacuum-assisted biopsies, and wire localization biopsy.

A subject may undergo blood sample withdrawal. After centrifugation, white blood cells may be isolated from the blood sample. Next, the white blood cells may be divided into diseased cells and control cells.

A subject may collect their own biological samples. The biological sample may be collected at home and transported to the medical center or facility. The biological sample may also be collected at a medical center, for example, at a doctor's office, clinic, laboratory patient service center, or hospital. Methods of collection may comprise male patient ejaculation, subjects coughing up sputum, subjects collecting stool during toileting, urination, saliva swab, combination of saliva and oral mucosal transudate collected from the mouth, and sweat collected by a sweat simulation procedure.

Assaying may begin after a user inputs the biological sample. Assaying can comprise nucleic acid extraction from the biological sample. Nucleic acids may be extracted from a biological sample using various techniques. During nucleic acid extraction, cells may be disrupted to expose the nucleic acid by grinding or sonicating. Detergent and surfactants may be added during cell lysis to remove the membrane lipids. Protease may be used to remove proteins. Also, RNase may be added to remove RNA. Nucleic acids can also be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif); (2) stationary phase adsorption methods (U.S. Pat. No. 5,234,809; Walsh et al., 1991, which is entirely incorporated herein by reference); and (3) salt-induced nucleic acid precipitation methods (Miller et al., (1988), such precipitation methods being typically referred to as “salting-out” methods. Another example of nucleic acid isolation and/or purification includes the use of magnetic particles (e.g., beads) to which nucleic acids can specifically or non-specifically bind, followed by isolation of the particles using a magnet, and washing and eluting the nucleic acids from the particles. See e.g., U.S. Pat. No. 5,705,628, which is entirely incorporated herein by reference. The above isolation methods may be preceded by an enzyme digestion step to help eliminate unwanted protein from the sample, e.g., digestion with proteinase K, or other like proteases. See, e.g., U.S. Pat. No. 7,001,724, which is entirely incorporated herein by reference. RNase inhibitors may be added to the lysis buffer. For certain cell or sample types, it may be desirable to add a protein denaturation/digestion step to the protocol. Purification methods may be directed to isolate DNA, RNA (including but not limited to mRNA, rRNA, tRNA), or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after subsequent manipulation, such as to remove excess or unwanted reagents, reactants, or products.

### Identifying Somatic Mutations in a Biological Sample

In another aspect, the present disclosure provides a method for identifying one or more somatic mutations in a biological sample from a subject. A tumor biological sample and normal biological sample may be obtained from the subject. The tumor biological sample and the normal biological sample may be assayed to (i) obtain sequence information for a first nucleic acid sample and a second nucleic acid sample automatically obtained from the tumor biological sample and the normal biological sample, respectively, without any involvement from a user, and (ii) identify one or more other biological markers of a type different than the first nucleic acid sample and the second nucleic acid sample. The sequence information obtained for the first nucleic acid sample and the second nucleic acid sample may be compared to identify one or more genomic alterations in the tumor biological sample relative to the normal biological sample. One or more other biological markers previously identified and one or more genomic alterations previously identified may be used to identify one or more somatic mutations in the subject at an accuracy of at least about 90% as compared to a control.

A first nucleic acid sample from a tumor biological sample of the subject and the second nucleic acid sample from a normal biological sample of the subject may be obtained. Obtaining a biological sample can comprise receiving a biological sample from the tumor tissue sample of the subject, and (ii) a biological sample from the normal tissue sample of the subject. The first biological sample and the second biological sample may be assayed to identify one or more biological markers in the tumor tissue sample relative to the normal tissue sample to generate a set of biologic data for the subject. The first nucleic acid sample and the second nucleic acid sample may be indexed. The first nucleic acid sample may be obtained from cell-free DNA in the plasma.

Assaying biological samples may comprise comparing the normal biomolecules to the abnormal biomolecules. After a user inputs a biological sample, the assaying may begin. The assaying can comprise processing the biological sample or sequencing the biological sample without any involvement from the user. The profiles of at least one or more markers of a disease or condition may be compared. This comparison can be quantitative or qualitative. Quantitative measurements can be taken using any of the assays described herein. Assaying may comprise processing a biological sample and/or sequencing of the biological sample without any involvement from a user. For example, sequencing, direct sequencing, random shotgun sequencing, Sanger dideoxy termination sequencing, whole-genome sequencing, exome sequencing, transcriptome sequencing, cell-free DNA sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiDsequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. The sequencing may be whole genome sequencing, low pass whole genome sequencing, or targeted sequencing. The sequencing may be whole transcriptome sequencing on RNA, such as tumor RNA.

Sequencing may also comprise detecting the sequencing product using an instrument, for example but not limited to an ABI PRISM 377 DNA Sequencer, an ABI PRISM 310, 3100, 3100-Avant, 3730, or 373OxI Genetic Analyzer, an ABI PRISM 3700 DNA Analyzer, or an Applied Biosystems SOLiD.™. System (all from Applied Biosystems), a Genome Sequencer 20 System (Roche Applied Science), or a mass spectrometer.

Sequencing can cover 2,500 genes, gene fusions, point mutations, indels, copy-number variations, promoters, and/or enhancers. Sequencing may be directed to at least 1 gene, 2 genes, 3 genes, 4 genes, 5 genes, 10 genes, 20 genes, 25 genes, 50 genes, 100 genes, 200 genes, 300 genes, 400 genes, or 500 genes, variants, or promoters thereof, selected from Table 1. Multiple subjects may be sequenced simultaneously. Sequencing may have a depth of coverage of at least about 0.5×, 1×, 2×, 3×, 4×, 5×, 6×, 7×, 8×, 9×, 10×, 20×, 30×, 40×, 50×, 100×, 200×, 300×, 400×, 500×, 600×, 700×, 800×, 900×, 1000×, 2000×, 3000×, 4000×, 5000×, 6000×, 7000×, 8000×, 9000×, or 10,000×. Sequencing can comprise whole exome sequencing, whole genome sequencing, or a combination thereof.

In a biological sample comprising one or more nucleic acids, various genes may be assayed. One or several, e.g., a panel, of genes may be assayed. For example, at least about 50 genes, 100 genes, 150 genes, 200 genes, 250 genes, 300 genes, or 500 genes may be assayed in the cell free DNA. The tumor biological sample may be a blood and formalin-fixed paraffin embedded (FFPE) tissue sample. The tissue sample may be frozen or fresh. The first nucleic acid sample and the second nucleic acid sample may be assayed for one or more genomic alterations and biomarkers at a concordance correlation coefficient of at least about 70%, 75%, 80%, 85%, 90%, 91%, 92%, 93%, 94%, 95%, 96%, 97%, 98%, or 99% when the first nucleic acid sample and the second nucleic acid sample are re-assayed for the presence or absence of the genomic alterations or biomarkers. The assayed genomic alterations and biomarkers may contain a plurality of genomic alterations and biomarkers. The genomic alterations may include a plurality of different types of genomic alterations. The genomic alterations may include: nucleotide insertions, nucleotide deletions, nucleotide substitutions, gene fusions, and copy-number variations, point mutations, gene amplifications, gene deletions, non-recurring mutations, and mRNA based alterations. At least 1 genomic alteration, 2 genomic alterations, 3 genomic alterations, 4 genomic alterations, 5 genomic alterations, 10 genomic alterations, 15 genomic alterations, 20 genomic alterations, 25 genomic alterations, 50 genomic alterations, or 100 genomic alterations may be identified at an accuracy of at least about 90%. For example, at least about 70%, 75%, 80%, 85%, 90%, 95%, or 99% accuracy.

Quantitative comparisons can include statistical analyses such as t-test, ANOVA, Kruskal-Wallis, Wilcoxon, Mann-Whitney, and odds ratio. Quantitative differences can include differences in the levels of markers between profiles or differences in the numbers of markers present between profiles, and combinations thereof. Examples of levels of the markers can be, without limitation, gene expression levels, nucleic acid levels, protein levels, lipid levels, and the like. Qualitative differences can include, but are not limited to, activation and inactivation, protein degradation, nucleic acid degradation, and covalent modifications.

The profile may be a nucleic acid profile, a protein profile, a lipid profile, a carbohydrate profile, a metabolite profile, immunohistochemistry profile, or a combination thereof. The profile can be qualitatively or quantitatively determined.

A nucleic acid profile can be, without limitation, a genotypic profile, a single nucleotide polymorphism profile, a gene mutation profile, a gene copy number profile, a DNA methylation profile, a DNA acetylation profile, a chromosome dosage profile, a gene expression profile, or a combination thereof.

The nucleic acid profile can be determined by various methods for determining or detecting genotypes, single nucleotide polymorphisms, gene mutations, gene copy numbers, DNA methylation states, DNA acetylation states, chromosome dosages. Biological markers may comprise antigens or genomic alterations. Biological markers may include one or more nucleotide insertions, nucleotide deletions, nucleotide substitutions, amino acid insertions, amino acid deletions, amino acid substitutions, gene fusions, copy-number variations, and any combination thereof.

Several methods or techniques can be used to analyze various biomolecules. Exemplary methods may include, but are not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. These methods for analysis may be wholly or partially automated and have varying degrees of user involvement.

The biological sample may be re-assayed at a later point in time and a change may be identified in one or more biological markers. The biological sample may be re-assayed in least about 30 minutes, 1 hours, 2 hours, 3 hours, 4 hours, 5 hours, 6 hours, 12 hours, 1 day, 2 days, 3 days, 5 days, 1 week, 2 weeks, 1 month, 6 months, 12 months, 1.5 years, 2 years, 5 years, 10 years, 20 years, 30 years, or 50 years. Assaying may comprise assaying at least about 50 biological markers, 100 biological markers, 150 biological markers, 200 biological markers, 250 biological markers, 300 biological markers, or 350 biological markers in a cell-free DNA or the biological sample.

### Methods of Processing Biological Sample

Various components can be isolated from a biological sample. A biological sample may comprise one or more cells and/or biomolecules, e.g., nucleic acids, proteins, hormones, and the like. Cell populations of the biological samples can be transformed into nucleic acids appropriate for molecular analysis. Target cells may be enriched from a heterogeneous cell population. The isolation process may be selected from laser-capture microdissection, gross dissection, or flow cytometry, among other techniques. Accompanying these processes is genetic manipulation to molecularly marked target cell types. Second, specific subsets of RNA and DNA may be extracted through direct, indirect, or modification protocols. A sequence library can be generated comprising DNA fragments labeled with a platform specific adaptor. The platform specific adaptor may be a sequence tag for sample indexing or molecular tagging.

Direct targeting DNA methods for sequence-specific enrichment may comprise molecular inversion probes, pulldown probes, bait sets, standard PCR, multiplex PCR, hybrid capture, endonuclease digestion, DNase I hypersensitivity, and selective circularization. Such probes may have sequences selected to target genes or sequences of interest, such as genes or variants thereof listed in Table 1. For example, such probes may have sequence complementarity with the genes or variants thereof listed in Table 1. RNA enrichment methods may be directed towards a specific subpopulation such as small RNAs or messenger ribonucleic acids (mRNAs). The RNA enrichment methods may be selected from, ‘not-so-random’ amplification, poly(A)-mediated reverse transcription, BrdU incorporation, or oligo(dT) hybridization. Strand preservation RNA enrichment methods may also include strand specific degradation after cDNA synthesis, orientation specific adaptor ligation, or reverse transcription-PCR of a specific biological target, or digestion of RNases for capturing secondary RNA structures. Enrichment can be achieved through negative selection of nucleic acids by eliminating undesired material. This sort of enrichment includes ‘footprinting’ techniques or ‘subtractive’ hybrid capture. During the former, the target sample is safe from nuclease activity through the protection of protein or by single and double stranded arrangements. During the latter, nucleic acids that bind ‘bait’ probes are eliminated.

DNA target enrichment may include in solution capture. During in solution capture, a custom pool of probes may be designed, synthesized and hybridized in solution to fragmented genomic DNA sample. The probes may be oligonucleotides and may be labeled with beads. The genomic DNA sample may be viral DNA present in the tumor sample. After the probes hybridize to the genomic regions of interest, the beads may be pulled down and washed. The beads can be removed and the genomic fragments may be sequenced in preparation for selective DNA sequencing of genomic sequences of interest. From the sequence reads, it can be determined which reads are off target and the probes that are associated with the off target reads. In the next cycle of in solution capture, the probes that correspond to the off target reads may be pulled down. The map of the off target reads, may compare the probes coverage. Then, the ratio of probes corresponding to off-target reads to on-target reads may be determined. The target rate for any set of probes may be estimated.

The probes may pull down at least about 1000 genes, 1500 genes, 2000 genes, 2500 genes, or 3000 genes. Once the desired or predetermined genes or genomic regions are selected, the probes may be synthesized. The probes may be at least about 50 nucleotides, 100 nucleotides, 150 nucleotides, 200 nucleotides, or 300 nucleotides in length. The probes may be separated into at least about 20 pools, 30 pools, 40 pools, 50 pools, 60 pools, 70 pools, 80 pools, 90 pools, or 100 pools. The probes may be separated based on biological function. The probes may be selected by their performance during sequencing. The assay may be conducted on a single probe level to identify which probes are selected. The probes may cover one or more coding regions, one or more non-coding regions, or both.

Nucleic acids can also be purified indirectly depending on their location to other molecular entities. The molecular entities may be other nucleic acids or proteins. The first step can be to form the desired cross-link types, such as DNA-DNA, DNA-protein, RNA-protein, or protein-protein. Cross-linkers may be selected from the group consisting of formaldehyde, ultraviolet (UV) light, dimethyl suberimidate (DMS), dimethyl adipimidate (DMA), glutaradehyde, bis(sulfosuccinimidyl) suberate (BS3), spermine or spermidine, and 1-ethyl-3-[3-dimethylaminopropyl]carbodiimide hydrochloride (EDAC). Immunoprecipitation can aid in nucleic acid extraction depending on their proximity to proteins of interests or histone modifications. Lastly, ligation may be another viable option in isolating co-localized nucleic acids to study chromosome interactions in the cell.

Modification protocols for nucleic acid extraction can direct transformation of the sequence to encode the specific modification. The protocols may include bisulfite treatment for detection of cytosine methylation and T4 bacteriophage b-glucosyltransferase and Huisgen cycloaddition for detection of 5-hydroxymethylcytosine. Post-transcriptional modifications of RNA may be detectable by determining the characteristic error signatures that they generate during the sequencing of data. Lastly, specific polymerase error signatures secondary to cross-linking events may be used to determine the target RNA nucleotide in RNA-protein interactions.

Prior to sequencing, the nucleic acids can be converted to a population of DNA fragments tagged with platform-specific adaptors. This tagging process may also occur after the nucleic acid targeting processes described above. “Fragment libraries” may first be created by random fragmentation. The fragmentation can be mechanical, chemical or enzymatic. After fragmentation, universal adaptor sequences can be ligated and undergo PCR amplification. For example, a hyperactive derivative of the Tn5 transposase can catalyze in vitro integration of the universal adaptor sequences into the target DNA at a high density. This is then usually followed by amplification. Another example PCR-free library preparation can minimize sequence bias. For example, sequencing technologies can choose to do without an amplification step.

The biological sample may be indexed. The biological sample may be tagged. A variety of methods can allow for many experiments to be efficiently multiplexed on a single sequencing lane. For example, a synthetic index or barcode may be flanked continually to all molecules in a sequencing library. The concurrent sequencing of the index can be used to determine reads in silico to the target libraries from which they derived. Alternatively, the sample may be tagged with a unique molecular index (UMI) which can be used for de-duplication at very a high coverage. Further, sequence may be appended that allows for mutations identification at deeper coverage, for example, detection of ultralow-frequency mutations by duplex sequencing. Synthetic tags can serve other functions. For example, individual molecules can be assigned during assembly. Accurate quantification, robust error-correction and increased effective read length may be achieved by categorizing reads from the same nucleic acid. Synthetic variants can be tagged during synthetic saturation mutagenesis and function as the readout. It may also be possible to assign tags to specific cells and determine genetic variability for single-cell resolution. The index may be or include a whole exome classifier.

The biological sample may comprise cell-free deoxyribonucleic acid (cfDNA) molecules, cellular deoxyribose nucleic acid (cDNA) molecules, ribonucleic acid (RNA) molecules, and protein, and wherein the cfDNA molecules, the cDNA molecules, and the RNA molecules are assayed for the presence or absence of the biological markers. The biological sample may comprise cfDNA. Dying tumor cells can release small pieces of their nucleic acids into a subject's bloodstream. These small pieces of nucleic acids are cell-free circulating tumor DNA (ctDNA).

Circulating tumor DNA can also be used non-invasively to monitor tumor progression and determine if a subject's tumor may react to targeted drug treatments. For example, the subject's ctDNA can be screened for mutations both before therapy and after therapy and drug treatment. During the therapy, developing somatic mutations can prevent the drug from working. For example, the subjects can observe an initial tumor response to the drug. This response can signal that the drug was initially effective in killing tumor cells. However, the development of new mutations may prevent the drug from continuing to work. Obtaining this critical information can assist doctors and oncologists in identifying that the subject's tumors are no longer responsive and different treatment is necessary. Circulating tumor DNA testing can be applicable to every stage of cancer subject care and clinical studies. Since ctDNA can be detected in most types of cancer at both early and advanced stages, it may be used as an effective screening method for most patients. A measurement of the levels of ctDNA in blood may also efficiently indicate a subject's stage of cancer and survival chances.

Various methods may be used to sequence cfDNA in addition to those discussed above. Techniques for sequencing cfDNA may include exome sequencing, transcriptome sequencing, genome sequencing, and cell-free DNA sequencing. Cell-free DNA sequencing may include mismatch targeted sequencing (Mita-Seq) and tethered elimination of termini (Tet-Seq).

In addition to sequencing, other reactions and/operations may occur within the systems and methods disclosed herein, including but not limited to: nucleic acid quantification, sequencing optimization, detecting gene expression, quantifying gene expression, genomic profiling, cancer profiling, or analysis of expressed markers. The assay may include immunohistochemistry profiling and genomic profiling of the biological sample. During immunohistochemistry, antigens may be identified during examination of the tumor and normal tissue cells of the biological sample. Immunohistochemistry can also provide results on the distribution and localization of biomarkers and differentially expressed proteins in different locations of the biological sample tissue. The differentially expressed proteins may be over or under-expressed proteins.

Genome profiling may be the process after sequencing in determining and measuring the activity of thousands of genes simultaneously. The profiling may be use to distinguish between cells that are actively dividing. Genomic profiling can also be used to measure how well cells respond to a particular treatment. One may determine patterns in the tumor DNA by comparing the tumor DNA against a set of known DNA. The group of genes whose combined expression pattern is uniquely characteristic to a given condition establishes the gene signature of the particular condition. The gene signature can then be used to choose a group of subjects at a specific state of a disease with accuracy that matches them with treatments.

### Identifying Genomic Aberrations and Custom Probes

In another aspect, the present disclosure provides a method for identifying a genomic aberration in one or more biological samples of a subject. Biological samples of the subject may be obtained and can comprise a nucleic acid sample that has or is suspected of having one or more genomic aberration(s) that appears at a frequency of less than about 1%, less than about 2%, less than about 3%, less than about 4%, less than about 5%, less than about 6%, less than about 7%, less than about 8%, less than about 9%, less than about 10%, less than about 15%, or less than about 20% in the nucleic acid sample. The nucleic acid sample may be enriched for a plurality of nucleic acid sequences to provide an enriched nucleic acid sample using a probe set comprising probes that have an on-target rate as a group of at least about 70%, at least about 75%, at least about 80%, at least about 85%, at least about 90%, and at least about 95%. The on-target rate as a group may be determined by (i) comparing the probe set to at least one predetermined region to measure (1) probe coverage of each probe in the probe set and (2) off-target probe coverage for each probe in the probe set, and (ii) determining the on-target rate of the probe set based on a ratio of the off-target coverage to the probe coverage. Alternatively, the off-target rate as a group may be determined by (i) comparing the probe set to at least one predetermined region to measure (1) probe coverage of each probe in the probe set and (2) on-target probe coverage for each probe in the probe set, and (ii) determining the off-target rate of the probe set based on a ratio of the on-target coverage to the probe coverage. The off-target probe coverage may measure the portion of probes that do not cover the predetermined region(s) of interest. The on-target probe coverage may measure the portion of probes that do cover the predetermined region(s) of interest. The probe coverage of each probe in the probe set may be the total mapped coverage of probes to the predetermined region(s) of interest. The enriched nucleic acid sample may then be sequenced to generate sequencing reads. The sequencing reads may be processed to identify one or more genomic aberration(s) in one or more biological samples of the subject that appears at a frequency of less than about 1%, less than about 2%, less than about 3%, less than about 4%, less than about 5%, less than about 6%, less than about 7%, less than about 8%, less than about 9%, less than about 10%, less than about 15%, or less than about 20% in the nucleic acid sample. One or more biological samples may comprise blood sample(s) and/or a tissue sample(s). The tumor tissue sample may be a FFPE tissue. One or more biological samples may be selected from the group consisting of protein, peptides, cell-free nucleic acids, ribonucleic acids, deoxyribose nucleic acids, and any combination thereof. One or more genomic aberrations can include nucleic acid mutations. One or more genomic aberrations may be selected from the group consisting of an insertion, nucleotide deletion, nucleotide substitution, amino acid insertion, amino acid deletion, amino acid substitution, gene fusion, copy-number variation, gene expression signatures, and any combination thereof.

The probe set can be further used to generate a classifier. First, one or more predetermined regions of a genome may be sequenced from a tumor tissue sample of the subject to provide sequencing reads. From the sequencing reads, sequences for the probe set may be identified that cover one or more predetermined regions of a genome. Then, the probe set may be compared to one or more predetermined regions to measure (i) probe coverage of each probe in the probe set and (ii) off-target probe coverage for each probe in the probe set. An on-target rate of the probe set may be determined based on a ratio of the off-target coverage to the probe coverage. A portion of the probe set may be selected that covers one or more predetermined regions of a genome and a portion of the probe set with an on-target rate as a group of at least about 50%, at least about 55%, at least about 60%, at least about 65%, at least about 70%, at least about 75%, at least about 80%, at least about 85%, at least about 90%, and at least about 95%, thereby determining a custom probe set. One or more features may be provided to permit classification of the probe set for one or more probes. Alternatively, the off-target rate as a group may be determined by (i) comparing the probe set to at least one predetermined region to measure (1) probe coverage of each probe in the probe set and (2) on-target probe coverage for each probe in the probe set, and (ii) determining the off-target rate of the probe set based on a ratio of the on-target coverage to the probe coverage.

One or more predetermined region(s) can comprise components selected from the group consisting of one or more segments of a gene, one or more segments of a plurality of genes, coding sequences, non-coding sequences, at least 2600 genes, gene fusions, point mutations, indels, copy-number variations, promoters, and/or enhancers. Such components may comprise at least about 500 genes, at least about 1000 genes, at least about 1200 genes, at least about 1400 genes, at least about 1600 genes, at least about 1800 genes, at least about 2000 genes, at least about 2200 genes, at least about 2600 genes, at least about 2800 genes, at least about 3000 genes, or at least about 3500 genes. One or more features can be selected from the group consisting of sequence, sequence length, alignment location, probe coverage, off-target probe coverage, on target rate, genomic aberrations, and genes or variants selected from Table 1. The predetermined regions may be coding or non-coding sequences. Non-coding sequences may comprise pseudogenes, genes for encoding RNA, introns and untranslated regions of mRNA, regulatory DNA sequences, repetitive DNA sequences, and transposons. Sequencing can be selected from the group consisting of exome sequencing, transcriptome sequencing, genome sequencing, and cell-free DNA sequencing.

The classifier may also provide a method for classifying a new set of probes. First, a classifier and a new probe set may be provided. Then, one or more features may be generated from the new set of probes. One or more features may be inputted from the new set of probes into the classifier. The classifier may be used to predict a classification outcome for the new set of probes. The features may be selected from the group consisting of sequence, sequence length, alignment location, probe coverage, off-target probe coverage, on target rate, genomic aberrations, and genes or variants selected from Table 1. The classification outcome can be selected from a choice of 0 or a choice of 1. The choice of 0 may indicate a selection to not order the new set of probes and the choice of 1 may indicate a selection to order the new set of probes. The classifier may be a machine learning algorithm. The classifier may be a supervised learning algorithm. The classifier may be a machine learning algorithm that is capable of getting trained by feature selection. Machine learning methods can be selected from the group consisting of decision tree learning, association rule learning, artificial neural networks, deep learning, inductive logic programming, support vector machines, clustering, Bayesian networks, reinforcement learning, representation learning, similarity and metric learning, sparse dictionary learning, genetic algorithms, rule-based machine learning, learning classifier systems, supervised learning, and unsupervised learning. In supervised machine learning, the pursuit for algorithms can reason from outwardly supplied instances to produce general hypotheses to determine predictions about future behavior. Supervised machine learning can build a succinct model of the distribution of class labels in terms of predictor features.

When generating a classifier, the classifier may be evaluated based on prediction accuracy. The accuracy may be determined by splitting a training set, by using a portion for estimating performance, by cross-validation, and leave-one-out validation. Examples of classification algorithms may include linear classifiers, support vector machines, quadratic classifiers, kernel estimation, boosting, decision trees, neural networks, FM NI neural networks, and learning vector quantization. Linear classifiers can include Fischer's linear discriminant, logistic regression, multinomial logistic regression, probit regression, support vector machines, Naïve Bayes classifier, and perceptron.

### Automated Sample Analysis Platforms

The present disclosure provides a system that may provide for analysis of one or more biological sample(s), which may be automated and/or not require involvement from a user. The automated system may preclude the need for any pipetting by a user, such as pipetting to transfer a sample from one station to another. For example, a user may input a biological sample into a machine for analysis of biocomponents (e.g., proteins and/or nucleic acids). Such an analyzer may analyze protein and/or nucleic acid biocomponents. The system, described in detail below, may provide a non-limiting example of an automated bioanalyzer that may not require any involvement from a user. The system may also comprise manual involvement from a user, such as manual pipetting.

The system may permit a user to prepare a biological sample for assaying and assay the biological sample without any pipetting by the user, or even without any involvement from the user. In some examples, the system permits the user to provide a biological sample (e.g., blood sample or tissue sample) to the system, at which point the system prepares the biological sample for sequencing and performs sequencing on the biological sample to generate sequencing data.

Systems of the present disclosure may permit a biological sample to be processed (e.g., sample preparation and sequencing) in a reproducible manner. For example, two systems as provided herein, in different geographic locations, may process the same biological sample or two subsets from the same biological sample and provide results that vary by at most about 10%, 9%, 8%, 7%, 6%, 5%, 4%, 3%, 2%, 1%, 0.1%, or 0.01%. Such variance may be determined, for example, by comparing sequence reads or consensus sequences.

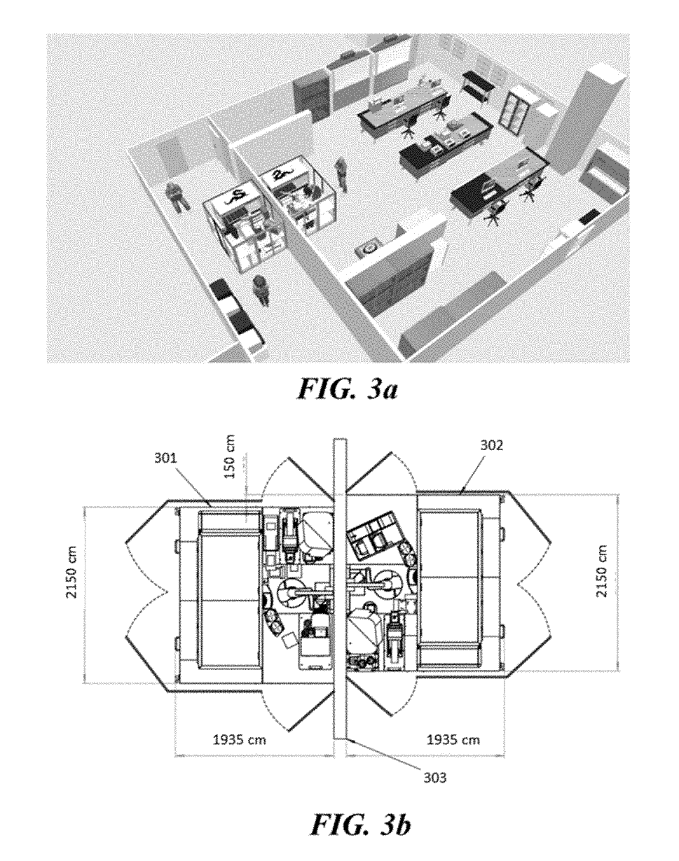

The system may comprise two robotic movers with at least about 20, 25, 30, 35, or 40 peripheral instruments. For example, the instruments may be selected from the group consisting of Spinnaker Robot with 1270 mm Extended Height Upgrade (Robotic Plate mover with gripper fingers and integrated camera), custom tables (Supports instruments and robotics), keyboard shelf and monitor stand (Support Keyboard and Monitor), Custom Guarding (Floor Standing Guarding), HEPA Ceiling with Positive Pressure (HEPA filtered air for pre PCR system with positive air pressure), HEPA Ceiling with Negative Pressure (Ceiling enclosure for Negative air pressure for Post Amplification system), Slide out Instrument Mezzanine (Pull out Mezzanine for instruments), Instrument Mezzanine (Fixed Instrument Mezzanine), Spinnaker Mix and Match Carousel (Plate Storage Carousel), Momentum Multimover (Scheduling Software with multi mover license), Momentum Concurrent License, Slide out Docking Tables (Custom Docking Tables for Hamilton Star), 10KVM UPS (Battery Backup), One Way Air Lock (Custom air lock between systems), AATI Fragment Analyzer (Performs QC on DNA fragments), ALPS 3000 (Plate Sealer (2 on system 2 offline)), Inheco Standard Plate Shaker (Automated Plate Shaker), Inheco DWP Plate Shaker (Automated Plate Shaker), Inheco Controller (Controls Plate Shakers), Inheco ODTC 96 (96 Well PCR Block), Hamilton Elite Decapper, Biotek MultifloFX (Dispenses Plates), Brooks Automation Xpeel (Plate Peeler), Thermo Kingfisher (DNA Extraction and Prep), Hamilton STAR (Liquid Handler), Bionex BeeSure (Acoustic Volume Check), Roche LC480 (QPCR), Bionex HiG4 (Plate Centrifuge), PCR Plate, Assay Plate for DNA Quantification, 96 Well Tube Racks, and 96 well tip boxes. The Hamilton STAR can be an automated liquid handler. The pre-Amplification STAR may be configured with 8 Pipetting channels, 2 Autolys channels (cell lysis and DNA extraction), EasyBlood Camera channel, and an Autoload barcode reader. The post-Amplification STAR can be configured with 8 Pipetting channels and an Autoload barcode reader. The EasyBlood component may be used in preparation and splitting of blood samples into their basic components including serum, plasma, white blood cells, and red blood cells. The camera may be used in determining the volume of separated plasma and cells. FIG. 3a shows a platform situated in a laboratory setting. FIG. 3b shows the system layout from above the wall of the laboratory between the two subunits. The system may comprise a Post-Amplification system 301 (left), a Pre-Amplification system 302 (right), and a separation wall 303. The instruments may be on mezzanines for compression or on pull our shelves for maintenance. Each subunit may be configured for pre-amplification steps or, separately, post-amplification steps. The system may comprise two subunits with a wall dividing the two. Each subunit may have a length of at least about 6 feet, 7 feet, 8 feet, 9 feet, or 10 feet and a width of at least about 6 feet, 7 feet, 8 feet, 9 feet, 10 feet, or 11 feet. The system may have a removable liquid handler (top) that rolls out on wheels. The liquid handler may be a Hamilton Star. The Hamilton Star can lock in place with embedded magnets to enable rapid instrument exchange. The two systems may be connected by a one way airlock prevents contamination of the pre-amplification system. The airlock may operate in conjunction with the Pre and Post air systems. Both sides of the system may have the Nexus XPeel and the ALPS3000 Plate sealer. The Beesure and Fragment Analyzer can reside in the post system (left) and the Biotek MulfifloFX and Hamilton Capper may reside in the Pre system (right). Access to all instruments may be available via doors connected to the emergency stop system which can also trigger the airlock closure when opened. The view in FIG. 3 show the system without the ceiling panels above the Pre and Post Amplification systems.

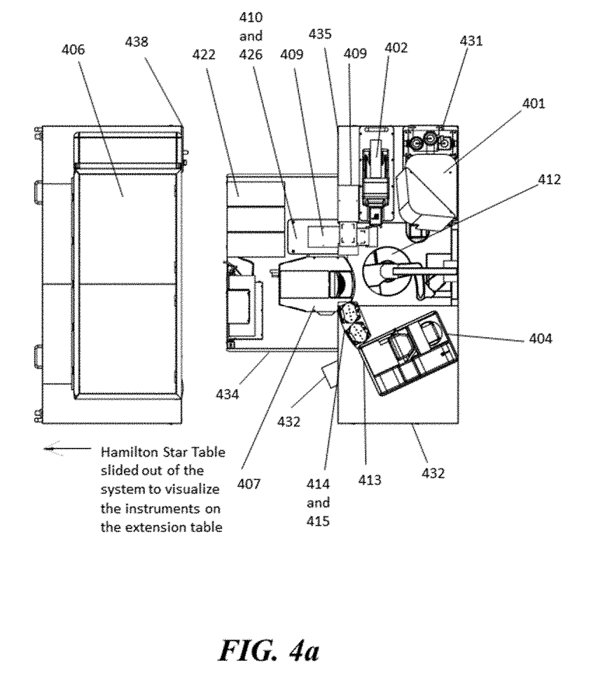

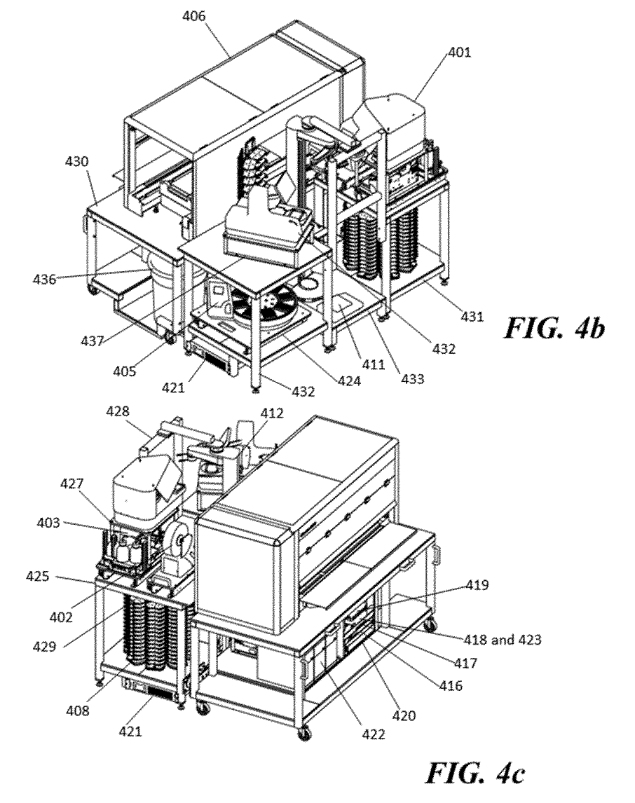

FIGS. 4a-c show several views of the Pre-Amplification system. The system may comprise an X-Peel seal peeler (Nexus X-Peel) 401, Abgene ALPS 3000 sealer 402, a microplate dispenser (Biotek Multiflow) 403, Hamilton Labelite Decapper 404, Thermo Kingfisher (DNA Extraction and Prep) 405, Hamilton Star 406, Bionex HiG4 centrifuge 407, carousel 408, Inheco incubator shaker 409, Inheco ODTC 410, balance 411, Spinnaker arm 412, Orbitor Randlom Access Hotel-8 shelf 413, 2 Position Hotel mount base 414, ORS2, Hotel Mounting Puck Assy 415, Moxa NPort 16-Port device server 416, Blackbox network HUB 417, general purpose input output (GPIO) box 418, mini hub 419, Inheco ODTC Controller 420, APC RACKMOUNT UPS 421, Dell desktop PC 422, rack mount bracket for the GPIO box 423, Slide Assembly, 26in 424/425/429, Mezz. Assy, 2 Lever, 440×460 426/427/437, frame for situating the mover only assembly arm 428, Hamilton Star docking table 430, Sealer Peeler custom table 431, Thermo Kingfisher custom table 432, SPNKR platform 433, extension platform for the Hamilton Star table 434, docking cart for pneumatic magnet plate assembly 435, 20 gallon bin for waste 436, and S-MAS4735-320-00 (438). FIG. 4a is the top view with the Hamilton Star table capable of sliding out of the system to visualize the instruments on the extension table. FIG. 4b and FIG. 4c are left and right views of the system.

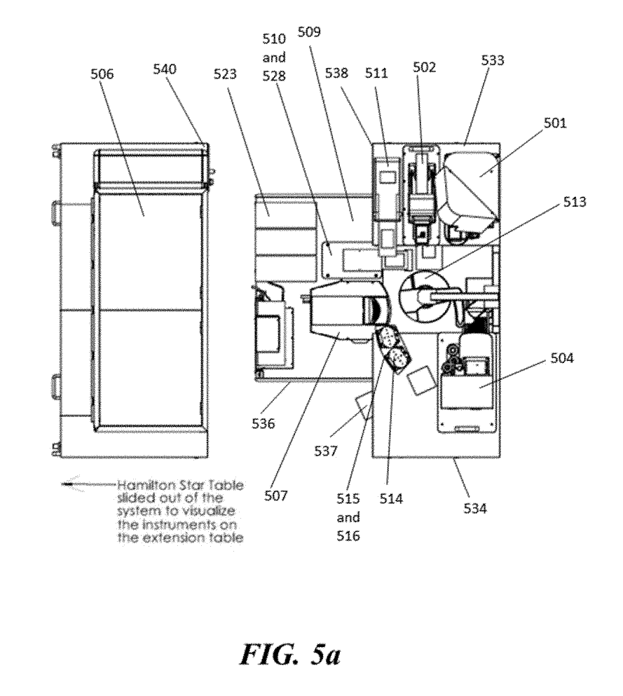

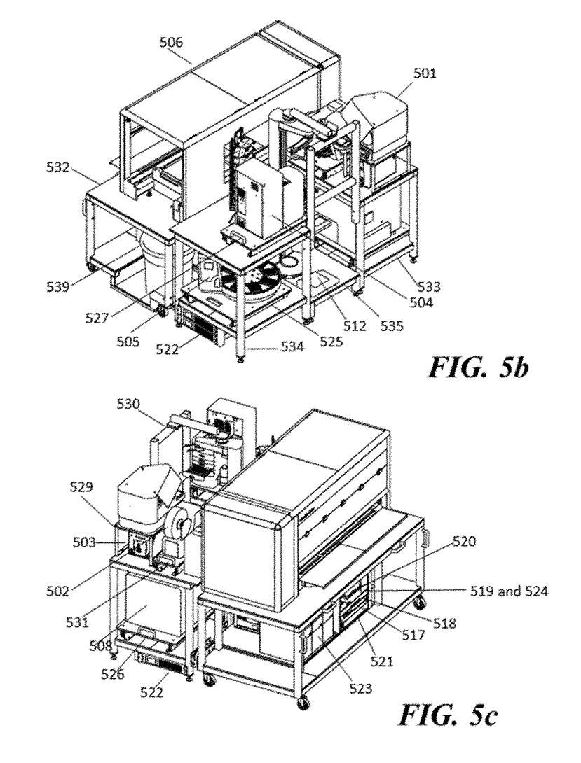

FIGS. 5a-c show several views of the Post-Amplification System. The system may comprise an X-Peel seal peeler 501, Abgene ALPS 3000 sealer 502, Bionex Beesure sensing system 503, Infinity fragment analyzer 504, Thermo Kingfisher 505, Hamilton Star 506, Bionex HiG4 centrifuge 507, PCR amplification and detection instrument (Roche Lightcycler 480) 508, Inheco microplate shaker 509, Inheco ODTC 510, Ultravap Mistral 511, balance 512, Spinnaker mover only assembly arm 513, Orbitor Randlom Access Hotel-8 shelf 514, microplate mover mount base 515, Hotel Mounting Puck Assy 516, Moxa NPort 16-port device server 517, blackbox network hub 518, GPIO box 519, Mini Hub 520, Inheco ODTC Controller 521, APC rackmount uninterrupted power supplies 522, Dell desktop PC 523, GPIO box rack mount bracket 524, Slide Assembly, 26in 525/526/527/531, mezzanine, 440×460 528 and 529, mover assembly arm support frame 530, Hamilton Star docking table 532, PCR amplification and detection instrument custom table 533, Thermo Kingfisher custom table 534, SPNKR platform 535, extension platform for the Hamilton Star table 536, waste chute 537, docking cart for pneumatic magnet plate assembly 538, 20 gallon bin 539, and S-MAS4735-320-00 (540). FIG. 5a is the top view with the Hamilton Star table capable of sliding out of the system to visualize the instruments on the extension table. FIG. 5b and FIG. 5c are left and right views of the system.

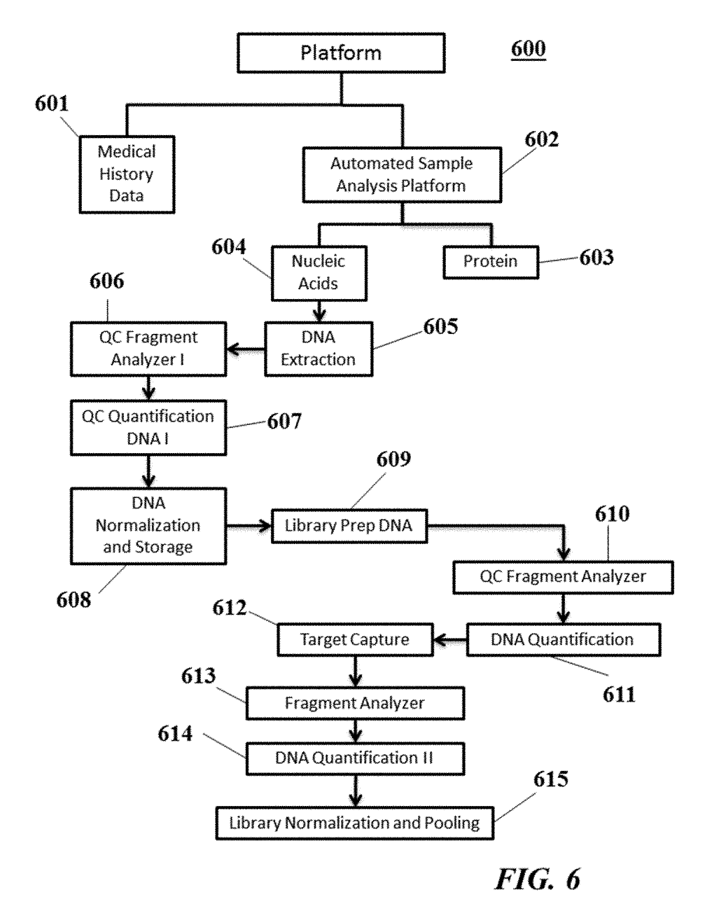

Assaying may begin after a user inputs the biological sample. A request from the user may be received to process the biological sample or sequence the biological sample. The process may be automated. FIG. 6 shows a schematic of a platform 600 for analysis of medical history and biological samples that can comprise an input for the subject's medical history 601 and input for biological samples into the automated sample analysis platform 602. The platform 600 may be open source. The automated sample analysis platform may receive biological samples. The biological sample may be nucleic acids 604 or protein 603. An automated sample analysis platform may be used to isolate biomolecules from the biological sample and deliver for sequencing. This process from start to finish may be automated. Blood sample in a tube and one or more slices from an FFPE tumor biopsy may be inserted into the system. During an initial quality control check, the amount of blood in the input tube may be validated. DNA, RNA or both from the blood sample may be extracted 605 from the white blood cells and the cell free DNA in the plasma. DNA and/or RNA can be extracted 605 from the tumor biopsy. The platform of FIG. 6 can include whole exome sequencing, whole genome sequencing, or a combination thereof.

During the quality check fragment analysis 606, the distribution size for biological sample's DNA fragments may be analyzed. The distribution size (or size distribution) may be at least about 100 base pairs (bp), 200 bp, 300 bp, 400 bp, 500 bp, 600 bp, 700 bp, 800 bp, 900 bp, 1000 bp, 1500 bp, or 2000 bp. Such size distribution may be an average or mean size distribution. The distribution size for FFPE tumor fragments may be at least about 50 bp, 100 bp, 150 bp, 200 bp, or 250 bp. The distribution size for cell free fragments may be at least about 50 bp, 100 bp, 150 bp, 200 bp, 250 bp. The distribution size for buffy coat fragments may be at least about 10 kb, 15 kb, 20 kb, 25 kb, 30 kb, 35 kb, or 40 kb. The isolated DNA may then be quantified 607 and the DNA concentration may be adjusted for storage 608. The FFPE tumor DNA quantified may be at least about 1 nanogram/microliter (ng/μL), 5 ng/μL, 10 ng/μL, 15 ng/μL, 20 ng/μL, 25 ng/μL, 30 ng/μL, 35 ng/μL, 40 ng/μL, 45 ng/μL, or 50 ng/μL. The cell free DNA quantified may be at least about 10 picograms/microliter (pg/μL), 20 pg/μL, 30 pg/μL, 40 pg/μL, 50 pg/μL, 60 pg/μL, 70 pg/μL, 80 pg/μL, 90 pg/μL, 100 pg/μL, 200 pg/μL, 300 pg/μL, 400 pg/μL, 500 pg/μL, 600 pg/μL, 700 pg/μL, 800 pg/μL, 900 pg/μL, 1000 pg/μL, or 1.5 ng/μL. The buffy coat DNA quantified may be at least about 1 ng/μL, 2 ng/μL, 3 ng/μL, 4 ng/μL, 5 ng/μL, 6 ng/μL, 7 ng/μL, 8 ng/μL, 9 ng/μL, 10 ng/μL, 15 ng/μL, 20 ng/μL, 25 ng/μL, 50 ng/μL, 100 ng/μL, 150 ng/μL, 200 ng/μL, or 300 ng/μL .During the DNA library preparations for downstream processes, the DNA fragments can be modified 609. The fragments can then undergo a quality control fragment analysis 610 by determining the distribution sizes for the modified DNA fragments and quantifying 611 the modified DNA. The distribution size (or size distribution) for FFPE tumor fragments may be at least about 50 bp, 100 bp, 150 bp, 200 bp, 250 bp, or 300 bp. The distribution size for buffy coat fragments may be at least about 50 bp, 100 bp, 150 bp, 200 bp, 300 bp, 400 bp, 500 bp, 600 bp, 700 bp, 800 bp, 900 bp, or 1000 bp. The FFPE tumor fragment quantified may be at least about 500 ng/μL, 600 ng/μL, 700 ng/μL, 800 ng/μL, 900 ng/μL, 1000 ng/μL, 1500 ng/μL, or 2000 ng/μL. The buffy coat fragment quantified may be at least about 500 ng/μL, 600 ng/μL, 700 ng/μL, 800 ng/μL, 900 ng/μL, 1000 ng/μL, 1500 ng/μL, or 2000 ng/μL. The cell free fragment quantified may be at least about 5 ng/μL, 10 ng/μL, 15 ng/μL, 20 ng/μL, 25 ng/μL, 30 ng/μL, 35 ng/μL, 40 ng/μL, 45 ng/μL, or 50 ng/μL. Of the DNA library, during target capture 612, DNA can be selected based on its match with at most about 1000 genes, 1500 genes, 2000 genes, 2500 genes, or 3000 genes in table 1. After target capture, the distribution of the size for the DNA fragments and the amount of DNA isolated may be measured 613, 614. Then, the DNA can be adjusted 615 to the correct concentration and each patient library can be tagged 615 with a specific barcode for downstream analysis. The correct concentration may be at most about 100 ng/μL, 150 ng/μL, 200 ng/μL, 250 ng/μL, 300 ng/μL, 350 ng/μL, 400 ng/μL, 450 ng/μL, 500 ng/μL, 550 ng/μL, or 600 ng/μL.

The system can accommodate at most about 100, 50, 45, 40, 35, 30, 20, 10, or less subject (e.g., patient) samples. Alternatively, the system can accommodate at least about 1, 2, 3, 4, 5, 6, 7, 8, 9, 10, 20, 30, 40, 50, 60, 70, 80, 90, 100 or more subject samples. Oligonucleotides, such as DNA or RNA (e.g., transcripts), can be selected for targets of interest, such as by enriching, and prepared for loading onto a nucleic acid sequencer (e.g., sequencer by Illumina, Pacific Biosciences of California, Ion Torrent or Oxford Nanopore). Each sample can be indexed and each indexed group can load together to the sequencer without mixing the results.

Polynucleotides may be tagged with a multitude of polynucleotide molecules from an adaptor library to generate a pool of tagged polynucleotides. The pool of tagged polynucleotides may be amplified among a variety of sequencing adaptors. The sequencing adaptors may comprise primers with sequences that are specifically complementary to sequences in of the plurality of polynucleotide molecules. Each of the sequencer adaptors may further contain an index tag, which can be a recognizable sample motif.

Tags can be any types of molecules chemically attached to aid in detection or labeling. Tags may be attached to a polynucleotide, comprising, nucleic acids, chemical compounds, florescent probes, or radioactive probes. Tags may also be oligonucleotides (e.g., DNA or RNA). Tags can comprise known sequences, unknown sequences, or both. A tag can comprise random sequences, pre-determined sequences, or both. A tag can be double-stranded or single-stranded. A double-stranded tag can be a duplex tag. A double-stranded tag can comprise two complementary strands. Alternatively, a double-stranded tag can comprise a hybridized portion and a non-hybridized portion. The double-stranded tag can be Y-shaped, e.g., the hybridized portion is at one end of the tag and the non-hybridized portion is at the opposite end of the tag. One such example is the “Y adapters” used in Illumina sequencing. Other examples include hairpin shaped adapters or bubble shaped adapters. Bubble shaped adapters have non-complementary sequences flanked on both sides by complementary sequences.

Samples may be processed to include barcodes (e.g., sample barcode, molecular barcode) and functional sequences that may be used, for example, to permit use of a given sample of a nucleic acid sequence. In an example, such functional sequences may include flow cell sequences that permit a nucleic acid sample to be coupled to a flow cell of a nucleic acid sequencer (e.g., Illumina P5/P7 adaptors).

A variety of methods can be used for tagging. For example, a polynucleotide can be tagged with an adaptor by hybridization. The adaptor may have a nucleotide sequence that is complementary to at least a portion of a sequence of the polynucleotide. The polynucleotide may also be tagged with an adaptor by ligation.

One or more enzymes may also be used for tagging. The enzyme can be a ligase such as a DNA ligase or a thermostable ligase. For example, the DNA ligase can be selected from a group consisting of E. coli DNA ligase, T4 DNA ligase, and/or mammalian ligase. The mammalian ligase can be DNA ligase I, DNA ligase III, or DNA ligase IV. Tags can be ligated to a blunt-end of a polynucleotide by blunt-end ligation. Tags can also be ligated to a sticky end of a polynucleotide by sticky-end ligation. Efficiency of ligation can be increased by optimizing various conditions. Efficiency of ligation can be increased by optimizing the reaction time of ligation. For example, the reaction time of ligation can be less than about 12 hours, such as less than about 1, less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, or less than 20 hours.

The ligase concentration of the reaction may increase the efficiency of ligation. For example, the ligase concentration can be at least about 10 unit/microliter, at least 50 unit/microliter, at least 100 unit/microliter, at least 150 unit/microliter, at least 200 unit/microliter, at least 250 unit/microliter, at least 300 unit/microliter, at least 400 unit/microliter, at least 500 unit/microliter, or at least 600 unit/microliter. Efficiency can also be optimized by adding or varying the concentration of an enzyme suitable for ligation, enzyme cofactors or other additives, and/or optimizing a temperature of a solution having the enzyme. Efficiency can also be optimized by varying the addition order of various components of the reaction. The end of tag sequence can comprise dinucleotide to increase ligation efficiency. When the tag comprises a non-complementary portion (e.g., Y-shaped adaptor), the sequence on the complementary portion of the tag adaptor can comprise one or more selected sequences that promote ligation efficiency. Preferably such sequences are located at the terminal end of the tag. Such sequences can comprise 1 terminal base, 2 terminal bases, 3 terminal bases, 4 terminal bases, 5 terminal bases, 6 terminal bases, 7 terminal bases, 8 terminal bases, 9 terminal bases, 10 terminal bases, 11 terminal bases, or 12 terminal bases. Reaction solution with high viscosity (e.g., a low Reynolds number) can also be used to increase ligation efficiency. For example, solution can have a Reynolds number less than 3000, less than 2000, less than 1000, less than 900, less than 800, less than 700, less than 600, less than 500, less than 400, less than 300, less than 200, less than 100, less than 50, less than 25, or less than 10. Further, roughly unified distribution of fragments can be used to increase ligation efficiency. The roughly unified distribution of fragments can be a tight standard deviation. For example, the variation in fragment sizes can vary by less than 20%, less than 15%, less than 10%, less than 5%, or less than 1%. Tagging can also comprise primer extension, for example, by polymerase chain reaction (PCR). Tagging can also comprise any of ligation-based PCR, multiplex PCR, single strand ligation, or single strand circularization.

The tags may also comprise molecular barcodes. Molecular barcodes can be used to differentiate polynucleotides in a sample and may be different from one another. For example, molecular barcodes can have a difference between them that can be characterized by a predetermined edit distance or a Hamming distance. In some instances, the molecular barcodes herein have a minimum edit distance of 1, 2, 3, 4, 5, 6, 7, 8, 9, or 10. To further improve efficiency of conversion (e.g., tagging) of untagged molecular to tagged molecules, one preferably utilizes short tags. For example, a library adapter tag can be up to about 75, 70, 65, 60, 55, 50, 45, 40, or 35 nucleotide bases in length. A collection of such short library barcodes can include a number of different molecular barcodes, such as at least 2, 4, 6, 8, 10, 12, 14, 16, 18 or 20 different barcodes with a minimum edit distance of 1, 2, 3 or more.

As a result, a collection of molecules may comprise one or more tags. In some instances, some molecules in a collection can include an identifying tag (“identifier”) such as a molecular barcode that is not shared by any other molecule in the collection. For example, in some instances of a collection of molecules, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the molecules in the collection can include an identifier or molecular barcode that is not shared by any other molecule in the collection. A collection of molecules may be considered “uniquely tagged” if each of at least 95% of the molecules in the collection carries an identifier that is not shared by any other molecule in the collection (“unique tag” or “unique identifier”). A collection of molecules is considered to be “non-uniquely tagged” if each of at least 1%, at least 5%, at least 10%, at least 15%, at least 20%, at least 25%, at least 30%, at least 35%, at least 40%, at least 45%, or at least or about 50% of the molecules in the collection bears an identifying tag or molecular barcode that is shared by at least one other molecule in the collection (“non-unique tag” or “non-unique identifier”). Accordingly, in a non-uniquely tagged population no more than 1% of the molecules are uniquely tagged. For example, in a non-uniquely tagged population, no more than 1%, 5%, 10%, 15%, 20%, 25%, 30%, 35%, 40%, 45%, or 50% of the molecules can be uniquely tagged. Examples of tags and adaptors, which may be used with methods and systems of the present disclosure, are provided in U.S. Patent Publication Nos. 2016/0040229 and 2016/0046986, each of which is entirely incorporated herein by reference.

The estimated number of molecules in a sample can result in a number of different tags selected. In some tagging methods, the number of different tags can be at least the same as the estimated number of molecules in the sample. In other tagging methods, the number of different tags can be at least two, three, four, five, six, seven, eight, nine, ten, one hundred or one thousand times as many as the estimated number of molecules in the sample. In unique tagging, at least two times (or more) as many different tags can be used as the estimated number of molecules in the sample.

The molecules in the sample may be non-uniquely tagged. In such instances a fewer number of tags or molecular barcodes is used then the number of molecules in the sample to be tagged. For example, no more than 100, 50, 40, 30, 20 or 10 unique tags or molecular barcodes are used to tag a complex sample such as a cell free DNA sample with many more different fragments.

The polynucleotide can be fragmented prior to tagging either naturally or using other approaches, such as, for example, shearing. The polynucleotides can be fragmented by certain methods selected from the group consisting of mechanical shearing, passing the sample through a syringe, sonication, heat treatment (e.g., for 30 minutes at 90° C.), and/or nuclease treatment (e.g., using DNase, RNase, endonuclease, exonuclease, and/or restriction enzyme).

The polynucleotides fragments before tagging can comprise sequences of any length. For example, the length can be selected from the group consisting of at least 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1000, 1100, 1200, 1300, 1400, 1500, 1600, 1700, 1800, 1900, 2000 or more nucleotides in length. The polynucleotide fragments can be about the average length of cell-free DNA. For example, the polynucleotide fragments can comprise about 160 bases in length. The polynucleotide fragment can also be fragmented from a larger fragment into smaller fragments about 160 bases in length.

Tagged polynucleotides tagged may include cancer related sequences. The cancer-associated sequences can comprise single nucleotide variation (SNV), copy number variation (CNV), insertions, deletions, and/or rearrangements.

Nucleic acid barcodes with identifiable sequences comprising molecular barcodes may be used for tagging. For example, a plurality of DNA barcodes can comprise various numbers of sequences of nucleotides. A plurality of DNA barcodes having 2, 3, 4, 5, 6, 7, 8, 9, 10, 11, 12, 13, 14, 15, 16, 17, 18, 19, 20, 21, 22, 23, 24, 25, 26, 27, 28, 29, 30 or more identifiable sequences of nucleotides can be used. When attached to only one end of a polynucleotide, the plurality of DNA barcodes can produce 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different identifiers. Alternatively, when attached to both ends of a polynucleotide, the plurality DNA barcodes can produce 4, 9, 16, 25, 36, 49, 64, 81, 100, 121, 144, 169, 196, 225, 256, 289, 324, 361, 400 or more different identifiers (which is the ̂2 of when the DNA barcode is attached to only 1 end of a polynucleotide). In one example, a plurality of DNA barcodes having 6, 7, 8, 9 or 10 identifiable sequences of nucleotides can be used. When attached to both ends of a polynucleotide, they produce 36, 49, 64, 81 or 100 possible different identifiers, respectively. Samples tagged in such a way can be those with a range of about 10 ng to any of about 100 ng, about 1 μg, about 10 μg of fragmented polynucleotides, e.g., genomic DNA, e.g., cfDNA.

There are many ways a polynucleotide may be uniquely identified. For example, a polynucleotide can be uniquely identified by a unique DNA barcode. Any two polynucleotides in a sample are attached two different DNA barcodes. Alternatively, a polynucleotide can be uniquely identified by the combination of a DNA barcode and one or more endogenous sequences of the polynucleotide. For example, any two polynucleotides in a sample can be attached the same DNA barcode, but the two polynucleotides can still be identified by different endogenous sequences. The endogenous sequence can be on an end of a polynucleotide. For example, the endogenous sequence can be adjacent (e.g., base in between) to the attached DNA barcode. In some instances the endogenous sequence can be at least 2, 4, 6, 8, 10, 20, 30, 40, 50, 60, 70, 80, 90, or 100 bases in length. The endogenous sequence may be a terminal sequence of the fragment/polynucleotides to be analyzed. The endogenous sequence may be the length of the sequence. For example, a plurality of DNA barcodes comprising 8 different DNA barcodes can be attached to both ends of each polynucleotide in a sample. Each polynucleotide in the sample can be identified by the combination of the DNA barcodes and about 10 base pair endogenous sequence on an end of the polynucleotide. Without being bound by theory, the endogenous sequence of a polynucleotide can also be the entire polynucleotide sequence.

A barcode can comprise either a contiguous or non-contiguous sequences. A barcode that comprises at least 1, 2, 3, 4, 5 or more nucleotides may be a contiguous sequence or non-contiguous sequence. For example, if a barcode comprises the sequence TTGC, a barcode is contiguous if the barcode is TTGC. On the other hand, a barcode is non-contiguous if the barcode is TTXGC, where X is a nucleic acid base.

An identifier or molecular barcode can have an n-mer sequence which may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. A tag herein can comprise any range of nucleotides in length. For example, the sequence can be between 2 to 100, 10 to 90, 20 to 80, 30 to 70, 40 to 60, or about 50 nucleotides in length.

The tag can comprise downstream of the identifier or molecular barcode, a double-stranded fixed reference sequence. The tag may also comprise a double-stranded fixed reference sequence upstream or downstream of the identifier or molecular barcode. Each strand of a double-stranded fixed reference sequence can be, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides in length.

These instruments may be used to perform the function described below: Hamilton STAR, Thermo KingFisher, Bionex HiG4 centrifuge, Inheco ODTC thermocycler, Inheco incubator shaker, Biotek MultifloFX, Thermo Fisher Spinnaker robotic arm, Thermo Fisher ALPS3000 plate sealer, Brooks XPeel, Roche LightCycler 480 for qPCR based nucleic acid quantitation, AATI Fragment Analyzer Infinity for nucleic acid size and quantity determination, and Hamilton LabElite Capper/Decapper. The automated sample analysis platform may perform multiple functions for biological sample analysis. These functions may include the main sample prep for the system (the Main method) and may be divided into two methods. The first method may include the Pre-Amplification Sample Processing which is associated with sequencing preparations. Pre-Amplification Sample Processing may comprise the tasks of DNA extraction from buffy coat or whole blood, cell-free DNA extraction from plasma, DNA and RNA extraction from FFPE tissues samples, DNA and RNA quantitation, QC, Normalization, DNA Fragmentation, End Repair, adapter Ligation and Bead Cleanup, PCR amplification and sample combination. Methods may vary in accordance with user preference(s). The system may have at least about 1 iteration, 2 iterations, 3 iterations, 4 iterations, or 5 iterations in a work day. One work day may be at least about 6 hours, 7 hours, 8 hours, 9 hours, or 10 hours. During each work day, at least about 1 PCR plate, 2 PCR plates, 3 PCR plates, 4 PCR plates, or 5 PCR plates may be transferred to Post-Amplification System. During the Pre-Amplification sample processing, the lysis method may be run on the liquid handler (Hamilton Star) with deep well plate. The tip box can be sent to the waste. The plate may be sealed and incubated for at least about 15 minutes, 30 minutes, 1 hour, 2 hours, or 3 hours with shaking. Then the plate may be undergo centrifugation for at least about 30 seconds, 1 minute, 1.5 minutes, 2 minutes, 3 minutes or 5 minutes. The plate may be peeled. The beads can be added onto the liquid handler and loaded onto the DNA and extraction prep shelves (Kingfisher). The beads may be magnetic beads. The extraction protocol ran and may comprise an additional wash and extraction of plates onto the Kingfisher. The extracted DNA may have magnetic heads. The QC plates on the fragment analyzer may be read. Sounds waves maybe utilized to determine the volume of fragments. If the samples are good, the result may include pure DNA or RNA from various samples. Quantification may be determined by capillary based separation of DNA by size. Real time or quantitative PCR (qPCR) may be used to measure the amount. The quantitative PCR may performed by a KAPA kit. The qPCR may be used to select for the DNA that will be sequenced. If the samples are bad, the extraction protocol can be re-run. The destination tube rack may be decapped and placed on the star deck. The data from the fragment analyzer and LightCycler 480 may be used to make the normalization plate on the Star. The sample may be aliquoted to the tube rack, re-capped, and sent to the output rack. During shearing, enzyme may be dispensed to the normalized plate. During shearing, flow cell adaptors may be attached to DNA. For cell free DNA, identifiers may be attached. The identifier may be a patient identifier or a unique identifier. The normalized plate may be sealed and incubated with shaking for at least about 10 minutes, 15 minutes, 20 minutes, 25 minutes, 30 minutes. The plate can be spun and the seal peeled. The end repair method can be run on the Star. The plate on the fragment analyzer may be read for QC. The normalized plate may be sealed and incubated with shaking for at least about 1 minutes, 5 minutes, 10 minutes, 20 minutes, 30 minutes, 1 hour, 2 hours, 3 hours, 4 hours, or 5 hours. The normalized plate may undergo centrifugation and then peeled. During adaptor ligation, the method may be run on the Star and beads can be added. The plate may be moved to Kingfisher and can undergo an additional wash and cleanup and eluent step. The magbead cleanup process can be run on the Kingfisher. The remaining plates may be removed to the waste or carousel from Kingfisher and the PCR plate may be sealed.

The completion time may be at least about 3 hours, 4 hours, 5 hours, 6 hours, 7 hours, 8 hours, 9 hours, or 10 hours for at least about 1 plate, 2 plates, 3 plates, 4 plates, 5 plates, 6 plates, or about 7 plates. The timing may be influenced by incubations that are at least about 30 min, 1 hr, 2 hrs, 3 hrs, 4 hrs, 5 hrs, or 10 hrs.

The second method may be the Post Amplification Plate preparation. The second method may include PCR, cleanup, QC, target capture, normalization and pooling. And these methods may change depending on the customer. During the Post Amplification Plate preparation, the Pre Amplification PCR plate may be placed on the Inheco and the protocol may be run. The PCR plate may be centrifuged and peeled, moved to the Star and transferred to the new Kingfisher plate. The reagents may be dispensed on the Biotek MultifloFX dispenser and transferred to the Kingfisher. The wash plates may be loaded, Kingfisher routine can be run, and transferred to the Star. The QC plate and PCR plate can be made. The beads can be added with Star, the Kingfisher routine can be run, transferred to the Star, and 8 PCR plates can be generated. The PCR protocol can then run, the Ampure cleanup protocol may be repeated on the Star and Kingfisher. The QC plate can be made, can run on the fragment analyzer, and the output and pool samples on the Star can be normalized. The system may also comprise a robotic camera that checks every plate and scans the barcode to ensure the right sample is handled.

The system providing for analysis of one or more biological sample(s) may be connected to a cloud computing system to form a “lab in a box with a cloud”. The cloud computing system may comprise a cloud storage system and one or more super computers. In cloud computing, a network of remote servers may be hosted on the internet to store, manage, and process data from the system providing for analysis of one or more biological sample(s), rather than a local server or a personal computer. In cloud storage, data and the mathematical models from the system providing for analysis of one or more biological sample(s) may be stored on remote servers accessed from the internet or “cloud”. The cloud storage may be maintained, operated and managed by a cloud storage service provider on storage servers that are built on virtualization methods. The output data and methods, disclosed herein, from the system providing for analysis of one or more biological sample(s) can transfer directly to the cloud computing system. The cloud computing system can comprise the system providing for analysis of one or more biological sample(s). The cloud computing system can store method and data as meta data along every step of the analysis of one or more biological sample(s). A user may have access to the “lab in a box with a cloud”.

### Biological Markers

The biological markers may include a plurality of different types of biological markers. In some cases, at least about 1 biological marker, 10 biological markers, 50 biological markers, 100 biological markers, 500 biological markers, 1000 biological markers, 1500 biological markers, 2000 biological markers, 2500 biological markers, 3000 biological markers, 3500 biological markers, or 4000 biological markers can be assayed. Through curated clinical trials and drugs, an annotated set of biological markers may be generated.

Cell-free DNA may be assayed for one or more biomarkers in the following genes including: ABL1, AKT1, AKT2, AKT3, ALK, APC, AR, ARAF, ARID1A, ASXL1, ATM, ATR, AURKA, AURKB, AURKC, BAP1, BCL2, BRAF, BRCA1, BRCA2, BRD2, BRD3, BRD4, CCND1, CCND2, CCND3, CCNE1, CDH1, CDK12, CDK4, CDK6, CDKN1A, CDKN1B, CDKN2A, CDKN2B, CEBPA, CREBBP, CRKL, CSF1R, CTNNB1, DDR2, DNMT3A, EGFR, EPHA3, EPHAS, ERBB2, ERBB3, ERBB4, ERCC2, ERG, ERRFIL ESR1, ETV1, ETV4, ETVS, ETV6, EWSR1, EZH2, FBXW7, FGFR1, FGFR2, FGFR3, FLCN, FLT3, GATA3, GNA11, GNAQ, GNAS, GSTM1, HNF1A, HRAS, IDH1, IDH2, IGF1R, JAK2, JAK3, KDR, KEAP1, KIT, KMT2A, KRAS, MAP2K1, MAP2K2, MAP2K4, MAPK1, MAPK3, MCL1, MDM2, MDM4, MED12, MEN1, MET, MITF, MKI67, MLH1, MPL, MSH2, MSH6, MTOR, MYC, MYD88, NF1, NF2, NFE2L2, NFKBIA, NKX2-1, NOTCH1, NOTCH2, NPM1, NRAS, NTRK1, NTRK3, NUTM1, PDGFRA, PDGFRB, PGR, PIK3CA, PIK3CB, PIK3R1, PTCH1, PTEN, PTPN11, RAB35, RAF1, RARA, RB1, RET, RHEB, RHOA, RIT1, RNF43, ROS1, RSPO2, RUNX1, SMAD2, SMAD4, SMARCA4, SMARCB1, SMO, SRC, STK11, SYK, TERT, TET2, TMPRSS2, TP53, TSC1, TSC2, VHL, WT1, XPO1, ZNRF3, BTK, CD274, FOXL2, MYCN, PDCD1LG2, and VEGFA.

Biomarkers may comprise at least one present in one or more of the following exons 61E3.4, AAK1, AARS, AARS2, AATK, ABCB1, ABCC9, ABI1, ABL1, ABL2, AC099552.4, ACKR3, ACP1, ACSL3, ACSL6, ACSM2B, ACTA2, ACTB, ACTC1, ACTG1, ACTL6B, ACTR2, ACVR1, ACVR1B, ACVR1C, ACVR2A, ACVR2B, ACVRL1, ADAM10, ADAM29, ADAMTS10, ADAMTS16, ADAMTS2, ADAMTS20, ADCK1, ADCK2, ADCK3, ADCK4, ADCK5, ADCY1, ADORA2A, ADRB1, ADRB2, ADRBK1, ADRBK2, AES, AFAP1, AFF1, AFF3, AFF4, AGBL4, AGXT2, AHCTF1, AHCYL2, AHDC1, AHNAK, AHNAK2, AJUBA, AK9, AKAP1, AKAP13, AKAP9, AKR1B10, AKT1, AKT2, AKT3, AL603965.1, ALDH2, ALDH3A2, ALDH7A1, ALG10B, ALK, ALKBH2, ALKBH3, ALOX12B, ALOX5, ALPK1, ALPK2, ALPK3, AMER1, AMHR2, AMPH, ANAPC1, ANKK1, ANKRD11, ANKRD12, ANKRD20A4, ANKRD30A, ANKRD36, ANKRD53, ANKRD6, ANXA6, ANXA8L2, AP003733.1, AP2A1, APAF1, APC, APC2, APEX1, APEX2, APIS, APLF, APOB, APOBEC3G, APTX, AQP12A, AQP7, AR, ARAF, AREG, ARFRP1, ARG1, ARG2, ARHGAP26, ARHGAP32, ARHGAP35, ARHGAP36, ARHGEF12, ARHGEF18, ARHGEF35, ARHGEF6, ARID1A, ARID1B, ARID2, ARID3A, ARID3B, ARID4A, ARID4B, ARID5A, ARID5B, ARNT, ASB5, ASCL4, ASH2L, ASPM, ASPSCR1, ASTN2, ASXL1, ASXL2, ASXL3, ATF1, ATF7IP, ATG13, ATG5, ATIC, ATM, ATP1A1, ATP2B3, ATR, ATRIP, ATRX, ATXN1, AURKA, AURKB, AURKC, AXIN1, AXIN2, AXL, B2M, B3GNTL1, B4GALT3, BAGE2, BAIAP2L1, BAP1, BARD1, BAZ1B, BAZ2A, BBC3, BCAP31, BCKDK, BCL10, BCL11A, BCL11B, BCL2, BCL2A1, BCL2L1, BCL2L11, BCL2L12, BCL2L2, BCL3, BCL6, BCL7A, BCL9, BCL9L, BCLAF1, BCOR, BCORL1, BCR, BIRC2, BIRC3, BLK, BLM, BMP2K, BMPR1A, BMPR1B, BMPR2, BMX, BPNT1, BRAF, BRCA1, BRCA2, BRD2, BRD3, BRD4, BRDT, BRINP3, BRIP1, BRSK1, BRSK2, BRWD3, BTG1, BTG2, BTK, BUB1, BUB1B, C11ORF30, C150RF65, C160RF59, C190RF40, C1ORF159, C1ORF86, C1QTNF5, C200RF26, C2CD3, C2ORF44, C3ORF70, C4ORF27, C7, C7ORF50, C7ORF55, CBA, C8ORF37, C8ORF44, CABLES2, CACNA1C, CACNA1D, CACNA1S, CAD, CALCR, CALM1, CALN1, CALR, CAMK1D, CAMK1G, CAMK2A, CAMK2B, CAMK2D, CAMK2G, CAMK4, CAMKK1, CAMKK2, CAMKV, CAMTAL CANT1, CARD11, CARM1, CARS, CASC5, CASK, CASP8, CAST, CBFA2T3, CBFB, CBL, CBLB, CBLC, CBLN4, CBWD1, CCAR1, CCDC107, CCDC144A, CCDC160, CCDC178, CCDC6, CCDC74A, CCNB HP1, CCND1, CCND2, CCND3, CCNE1, CCNH, CD163L1, CD274, CD276, CD40, CD5L, CD74, CD79A, CD79B, CD82, CDC14A, CDC14B, CDC20, CDC25A, CDC25B, CDC25C, CDC27, CDC42, CDC42BPA, CDC42BPB, CDC42BPG, CDC42EP1, CDC7, CDC73, CDH1, CDH10, CDH11, CDH18, CDH2, CDH2O, CDH4, CDH5, CDH6, CDH9, CDK1, CDK10, CDK11A, CDK12, CDK13, CDK14, CDK15, CDK16, CDK17, CDK18, CDK19, CDK2, CDK20, CDK3, CDK4, CDK5, CDK5RAP2, CDK6, CDK7, CDK8, CDK9, CDKL1, CDKL2, CDKL3, CDKL4, CDKL5, CDKN1A, CDKN1B, CDKN2A, CDKN2B, CDKN2C, CDKN3, CDX2, CEBPA, CEP170, CEP89, CETN2, CFH, CFHR4, CFLAR, CHAF1A, CHCHD7, CHD2, CHD3, CHD4, CHD5, CHD7, CHD8, CHDC2, CHEK1, CHEK2, CHIC2, CHMP3, CHN1, CHUK, CIC, CIITA, CIT, CKMT1A, CKS1B, CLCN6, CLDN18, CLIP1, CLK1, CLK2, CLK3, CLK4, CLP1, CLSTN2, CLTC, CLTCL1, CLVS2, CMKLR1, CNBD1, CNBP, CNOT1, CNOT3, CNPY3, CNTN1, CNTNAP5, CNTRL, COBLL1, COL11A1, COL18A1, COL1A1, COL1A2, COL2A1, COL3A1, COMT, COX6C, CPS1, CPXCR1, CR1, CRB1, CREB1, CREB3L1, CREB3L2, CREBBP, CRIPAK, CRKL, CRLF2, CRTC1, CRTC3, CSDE1, CSF1, CSF1R, CSF3R, CSK, CSNK1A1, CSNK1A1L, CSNK1D, CSNK1E, CSNK1G1, CSNK1G2, CSNK1G3, CSNK2A1, CSNK2A2, CTAGE6, CTCF, CTDNEP1, CTDSP1, CTDSP2, CTDSPL, CTDSPL2, CTLA4, CTNNA1, CTNNA2, CTNNB1, CTNND1, CTTN, CUL1, CUL3, CUX1, CXCR4, CYC 1, CYLD, CYP11B1, CYP2A6, CYP2B6, CYP2C19, CYP2C8, CYP2C9, CYP2D6, CYP3A4, CYP3A5, CYP4F2, DAB2IP, DACH1, DACH2, DAPK1, DAPK2, DAPK3, DAXX, DCAF12L2, DCC, DCLK1, DCLK2, DCLK3, DCLRE1A, DCLRE1B, DCLRE1C, DCP1B, DCTN1, DCUN1D1, DDB1, DDB2, DDIT3, DDR1, DDR2, DDX10, DDX3X, DDX5, DDX6, DEFB114, DEFB118, DEFB119, DEK, DERL1, DHX16, DHX9, DIAPHL DICER1, DIDO1, DI02, DIS3, DIS3L2, DISP1, DKK2, DKK4, DLG2, DLX4, DMC1, DMD, DMPK, DNAH12, DNAJA2, DNAJC6, DNER, DNM2, DNM3, DNMT1, DNMT3A, DNMT3B, DOCK2, DOCK4, DOK6, DOLPP1, DOT1L, DPH3, DPPA4, DPYD, DRD2, DRD5, DSC2, DSG2, DSP, DST, DSTYK, DUPD1, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, DUT, DYNCH1, DYRK1A, DYRK1B, DYRK2, DYRK3, DYRK4, E2F3, EBF1, EBPL, ECT2L, EDNRB, EED, EEF1A1, EEF2K, EGFL7, EGFR, EGR3, EIF1AX, EIF2AK1, EIF2AK2, EIF2AK3, EIF2AK4, EIF2S1, EIF3E, EIF4A2, ELAVL3, ELF3, ELF4, ELF5, ELK4, ELL, ELN, ELTD1, EME1, EME2, EMG1, EML4, ENDOV, EP300, EPAS1, EPB41L3, EPCAM, EPDR1, EPHA1, EPHA10, EPHA2, EPHA3, EPHA4, EPHA5, EPHA6, EPHA7, EPHA8, EPHB1, EPHB2, EPHB3, EPHB4, EPHB6, EPM2A, EPOR, EPPK1, EPS15, ERBB2, ERBB2IP, ERBB3, ERBB4, ERC1, ERCC1, ERCC2, ERCC3, ERCC4, ERCC5, ERCC6, ERCC6L, ERCC8, ERG, ERN1, ERN2, ERRFIl, ESPL1, ESR1, ESR2, ESRRG, ETNK1, ETS1, ETV1, ETV4, ETV5, ETV6, EWSR1, EXO1, EXOSC10, EXT1, EXT2, EYA1, EYA2, EYA3, EYA4, EZH1, EZH2, EZR, F2, F5, FADD, FAM101A, FAM129B, FAM129C, FAM131B, FAM155A, FAM157B, FAM174B, FAM175A, FAM194B, FAM21A, FAM46C, FAM46D, FAM58A, FAM71B, FAM83H, FAM86B1, FAM86B2, FAM9A, FAN1, FANCA, FANCB, FANCC, FANCD2, FANCE, FANCF, FANCG, FANCI, FANCL, FANCM, FANK1, FAS, FASTK, FAT1, FBN1, FBN2, FBX011, FBXO43, FBXW7, FCGR1A, FCGR2B, FCGR3B, FCHO2, FCRL4, FEN1, FER, FES, FEV, FGF10, FGF14, FGF19, FGF23, FGF3, FGF4, FGF6, FGF7, FGFR1, FGFR1OP, FGFR2, FGFR3, FGFR4, FGR, FH, FHIT, FIP1L1, FIS1, FKBP9, FLCN, FLI1, FLNA, FLT1, FLT3, FLT4, FN1, FNBP1, FOLR1, FOSL2, FOXA1, FOXA2, FOXL2, FOXO1, FOXO3, FOXO4, FOXP1, FOXP4, FOXQ1, FRG1, FRG2B, FRK, FRS2, FSCN3, FSIP1, FSTL3, FTH1, FUBP1, FUS, FUT9, FYN, G3BP1, G6PD, GAB2, GAB3, GABRA6, GABRB2, GABRB3, GABRP, GAK, GALNT13, GAS6, GAS7, GATA1, GATA2, GATA3, GATA4, GATA6, GATS, GCK, GCSAML, GDI1, GEN1, GID4, GIGYF2, GIPC3, GLA, GLI1, GLI2, GLIPR1L2, GML, GMPS, GNA11, GNA13, GNAIl, GNAQ, GNAS, GNL3L, GNPTAB, GOLGA2, GOLGA5, GOLGA6L6, GOPC, GOT2, GP6, GPC3, GPC6, GPHN, GPR124, GPR89A, GPRASP1, GPS2, GP SM1, GREM1, GRIN2A, GRIN3A, GRK4, GRK5, GRK6, GRK7, GRM3, GRXCR1, GSG2, GSK3A, GSK3B, GSTM1, GSTP1, GSTT1, GTF2H1, GTF2H2, GTF2H3, GTF2H4, GTF2H5, GTF2I, GTF3C5, GUCY1A2, GUCY2C, GUCY2D, GUCY2F, H1F0, H1FNT, H1FOO, H1FX, H2AFB1, H2AFB2, H2AFB3, H2AFJ, H2AFV, H2AFX, H2AFY, H2AFY2, H2AFZ, H2BFM, H2BFWT, H3F3A, H3F3B, H3F3C, HCK, HCN1, HDAC1, HDAC10, HDAC11, HDAC2, HDAC3, HDAC4, HDAC5, HDAC6, HDAC7, HDAC8, HDAC9, HDDC2, HDHD1, HDHD2, HDHD3, HECW1, HELQ, HERC1, HERC2, HERPUD1, HEY1, HGF, HHLA2, HIF1A, HIP1, HIPK1, HIPK3, HIPK4, HIST1H1A, HIST1H1B, HIST1H1C, HIST1H1D, HIST1H1E, HIST1H1T, HIST1H2AA, HIST1H2AB, HIST1H2AC, HIST1H2AD, HIST1H2AE, HIST1H2AG, HIST1H2AH, HIST1H2AI, HIST1H2AJ, HIST1H2AK, HIST1H2AL, HIST1H2AM, HIST1H2BA, HIST1H2BB, HIST1H2BC, HIST1H2BD, HIST1H2BE, HIST1H2BF, HIST1H2BG, HIST1H2BH, HIST1H2BI, HIST1H2BK, HIST1H2BL, HIST1H2BM, HIST1H2BO, HIST1H3A, HIST1H3B, HIST1H3C, HIST1H3D, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4I, HIST1H4J, HIST1H4K, HIST1H4L, HIST2H2AA3, HIST2H2AA4, HIST2H2AB, HIST2H2AC, HIST2H2BE, HIST2H3A, HIST2H3C, HIST2H3D, HIST2H4A, HIST3H2A, HIST3H2BB, HIST3H3, HKR1, HLA-A, HLA-B, HLF, HLTF, HMGA1, HMGA2, HMGXB4, HNF1A, HNRNPA2B1, HNRNPM, HOOK3, HOXA11, HOXA13, HOXA3, HOXA9, HOXB13, HOXC11, HOXC13, HOXD11, HOXD13, HPCAL4, HRAS, HS6ST1, HSD3B1, HSP9OAA1, HSP9OAA2P, HSP90AB1, HSPA2, HSPA5, HSPA8, HSPB8, HUNK, HUS1, HUWE1, IAPP, IARS2, ICK, ICOSLG, ID3, IDH1, IDH2, ID01, IFNGR1, IFNL3, IFT172, IGF1, IGF1R, IGF2, IGF2BP3, IGF2R, IGFBP7, IK, IKBKAP, IKBKB, IKBKE, IKBKG, IKZFL IKZF2, IKZF3, IL10, IL18RAP, IL1RAPL1, IL2, IL21R, IL2RG, IL3, IL32, IL36A, IL6ST, IL7R, ILF2, ILK, ILKAP, IMPA1, IMPA2, IMPAD1, ING1, INHBA, INPP1, INPP4A, INPP4B, INPP5A, INPP5B, INPP5D, INPP5E, INPP5F, INPP5J, INPP5K, INPPL1, INSR, INSRR, INTS1, INTS4, IRAK1, IRAK2, IRAK3, IRAK4, IRF2, IRF4, IRS1, IRS2, ISOC2, ITGA6, ITK, ITPA, ITPR1, ITPR3, JAK1, JAK2, JAK3, JARID2, JAZFL JMJD1C, JUN, KALRN, KANK3, KAT6A, KAT6B, KCNE1, KCNH2, KCNJ11, KCNJ5, KCNQ1, KCNT2, KDM5A, KDM5B, KDM5C, KDM6A, KDM6B, KDR, KDSR, KEAP1, KEL, KIAA1109, KIAA1549, KIAA1598, KIDINS220, KIF20B, KIF3A, KIF5B, KIFC3, KIT, KLF4, KLF5, KLF6, KLHL4, KLHL6, KLK2, KLRG1, KMT2A, KMT2B, KMT2C, KMT2D, KNSTRN, KRAS, KRT1, KRTAP1-1, KRTAP15-1, KRTAP19-6, KRTAP5-5, KSR1, KSR2, KTN1, LARS, LASP1, LATS1, LATS2, LCE1B, LCK, LCP1, LDLR, LEF1, LENG9, LEPR, LEPROTL1, LGI4, LHFP, LHPP, LHX9, LIFR, LIG1, LIG3, LIG4, LILRB5, LIMK1, LIMK2, LIN28A, LIN28B, LIN7A, LMNA, LMO1, LMO2, LMOD2, LMTK2, LMTK3, LPP, LPPR1, LPPR2, LPPR3, LPPR4, LPPR5, LRFN5, LRIG3, LRP1B, LRP6, LRRC4C, LRRC55, LRRIQ1, LRRIQ3, LRRK1, LRRK2, LRRTM4, LSM14A, LTBP1, LTBR, LTK, LTV1, LUC7L2, LUM, LUZP2, LYL1, LYN, LZTR1, MACF1, MAD2L2, MADCAM1, MAF, MAFB, MAGEA3, MAGEB18, MAGEB2, MAGEC1, MAGI2, MAK, MALT1, MAML2, MAP1A, MAP1B, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K2, MAP3K3, MAP3K4, MAP3K5, MAP3K6, MAP3K7, MAP3K8, MAP3K9, MAP4, MAP4K1, MAP4K3, MAP4K4, MAP4K5, MAPK1, MAPK10, MAPK11, MAPK12, MAPK13, MAPK14, MAPK15, MAPK3, MAPK4, MAPK6, MAPK7, MAPK8, MAPK8IP1, MAPK9, MAPKAPK2, MAPKAPK3, MAPKAPK5, 2-Mar, MARCKSL1, MARK1, MARK2, MARK3, MARK4, MAST1, MAST2, MAST3, MAST4, MASTL, MAT2A, MATK, MAX, MBD4, MCL1, MCMI, MCTP1, MDC1, MDM2, MDM4, MDN1, MECOM, MED12, MED13, MED16, MED17, MED20, MEF2A, MEF2B, MEF2C, MEGF6, MELK, MEN1, MERTK, MET, METRNL, METTL14, MGA, MGMT, MGRN1, MICAL1, MINPP1, MITF, MKI67, MKL1, MKNK1, MKNK2, MKRN1, MLF1, MLH1, MLH3, MLKL, MLLT1, MLLT10, MLLT11, MLLT3, MLLT4, MLLT6, MME, MMP2, MMP24, MMP9, MMS19, MN1, MNAT1, MNX1, MOK, MOS, MPG, MPL, MPLKIP, MPND, MPP7, MPRIP, MRAS, MRE11A, MROH2B, MRPS31, MRPS9, MSH2, MSH3, MSH4, MSH5, MSH6, MSI2, MSMB, MSN, MST1, MST1R, MST4, MTCP1, MTF2, MTHFR, MTM1, MTMR1, MTMR10, MTMR11, MTMR12, MTMR2, MTMR3, MTMR4, MTMR6, MTMR7, MTMR8, MTMR9, MTOR, MTRNR2L1, MTRNR2L8, MTUS2, MUC1, MUC2, MUC4, MUC6, MUC7, MUM1L1, MUS81, MUSK, MUTYH, MYB, MYBL1, MYBPC3, MYC, MYCBP2, MYCN, MYD88, MYH11, MYH7, MYH9, MYL10, MYL2, MYL3, MYLK, MYLK2, MYLK3, MYLK4, MYNN, MYO1D, MYO3A, MYO3B, MYO5A, MYOD1, MYOZ3, MYT1, NAA15, NAB2, NABP2, NACA, NACC2, NALCN, NAP1L2, NAT2, NAV1, NAV3, NBEA, NBN, NBPF10, NCF1, NCKIPSD, NCOA1, NCOA2, NCOA3, NCOA4, NCOA7, NCOR1, NCOR2, NDRG1, NEB, NEDD4L, NEFH, NEIL 1, NEIL2, NEIL3, NEK1, NEK10, NEK11, NEK2, NEK3, NEK4, NEK5, NEK6, NEK7, NEK8, NEK9, NELFA, NELFB, NF1, NF2, NFATC2, NFE2L2, NFE2L3, NFIB, NFKB1, NFKB2, NFKBIA, NFKBIB, NFKBIE, NFKBIZ, NHEJ1, NIM1, NIN, NIPBL, NKX2-1, NKX3-1, NLK, NLRP2, NLRP3, NLRP5, NLRP6, NM, NMS, NMT2, NOD1, NOMO1, NONO, NOTCH1, NOTCH2, NOTCH2NL, NOTCH3, NOTCH4, NPAS3, NPEPL1, NPEPPS, NPM1, NPR1, NPR2, NQO1, NR, NR1H2, NR4A2, NR4A3, NRAS, NRBP1, NRBP2, NRG1, NRG3, NRK, NSD1, NT5C2, NTHL1, NTM, NTNG1, NTRK1, NTRK2, NTRK3, NUAK1, NUAK2, NUDT1, NUDT10, NUDT11, NUDT14, NUDT3, NUDT4, NUMA1, NUMBL, NUP214, NUP93, NUP98, NUTM1, NUTM2A, NUTM2B, NXPE1, OBSCN, OCRL, OGG1, OLIG2, OMD, OR2L2, OR2W3, OR5L1, OR9G1, OSBPL6, OSR1, OTOL1, OTUB1, OTUD4, OXA1L, OXNAD1, OXR1, P2RY11, P2RY8, P4HB, PABPC1, PABPC3, PABPC4, PABPC5, PACS1, PADI2, PADI4, PAFAH1B2, PAK1, PAK2, PAK3, PAK4, PAK6, PAK7, PALB2, PAN3, PAPD5, PARK2, PARM1, PARP1, PARP2, PARP3, PASK, PATZ 1, PAX3, PAX5, PAX7, PAX8, PBK, PBRM1, PBX1, PCBP1, PCDH11X, PCK1, PCM1, PCMTD1, PCNA, PCSK7, PCSK9, PDCD1, PDCD1LG2, PDE1A, PDE4DIP, PDGFB, PDGFRA, PDGFRB, PDIK1L, PDK1, PDK2, PDK3, PDK4, PDP2, PDPK1, PDS5A, PDS5B, PDXP, PDYN, PEAK1, PEG3, PERI, PES1, PFN2, PGM5, PGP, PGR, PHF 1, PHF 19, PHF6, PHKG1, PHKG2, PHLDA1, PHLDA3, PHLPP2, PHOX2B, PICALM, PIK3C2B, PIK3C2G, PIK3C3, PIK3CA, PIK3CB, PIK3CD, PIK3CG, PIK3R1, PIK3R2, PIK3R3, PIK3R4, PIM1, PIM2, PIM3, PINK1, PIP5K1A, PJA1, PKD1, PKD2, PKDCC, PKHD1, PKN1, PKN2, PKN3, PKP2, PLAG1, PLAGL1, PLCG1, PLCG2, PLCH2, PLCL1, PLEC, PLEKHS1, PLK1, PLK2, PLK3, PLK4, PMAIP1, PML, PMS1, PMS2, PNCK, PNKP, PNLIPRP3, PNRC1, POLB, POLD1, POLE, POLG, POLH, POLI, POLK, POLL, POLM, POLN, POLQ, POLR2D, POM121L12, POMK, POT1, POTEC, POTEF, POTEG, POU2AF 1, POU3F2, POU5F 1, PPA1, PPA2, PPAP2A, PPAP2B, PPAP2C, PPAPDC1A, PPAPDC1B, PPAPDC2, PPAPDC3, PPARG, PPEF 1, PPEF2, PPFIA4, PPFIBP1, PPIF, PPM1A, PPM1B, PPM1D, PPM1E, PPM1F, PPM1G, PPM1H, PPM1J, PPM1K, PPM1L, PPM1M, PPM1N, PPP1CA, PPP1CB, PPP1CC, PPP2CA, PPP2CB, PPP2R1A, PPP3CA, PPP3CB, PPP3CC, PPP4C, PPP5C, PPP6C, PPTC7, PRB 1, PRB2, PRB4, PRCC, PRDM1, PRDM16, PRDM2, PRELID2, PREX2, PRF 1, PRG4, PRKAA1, PRKAA2, PRKACA, PRKACB, PRKACG, PRKAG2, PRKAR1A, PRKAR1B, PRKCA, PRKCB, PRKCD, PRKCE, PRKCG, PRKCH, PRKCI, PRKCQ, PRKCZ, PRKD3, PRKDC, PRKG1, PRKG2, PRKX, PRPF 19, PRPF4, PRPF8, PRRC2A, PRRX1, PRSS1, PRSS3, PRSS8, PRX, PSEN1, PSG5, PSG6, PSG8, PSIP1, PSKH1, PSKH2, PSMD11, PSME3, PSPH, PTCH1, PTCH2, PTEN, PTH, PTK2, PTK2B, PTK6, PTK7, PTP4A1, PTP4A2, PTP4A3, PTPDC1, PTPLA, PTPMT1, PTPN1, PTPN11, PTPN12, PTPN13, PTPN14, PTPN18, PTPN2, PTPN20A, PTPN21, PTPN22, PTPN23, PTPN3, PTPN4, PTPN5, PTPN6, PTPN7, PTPN9, PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZ1, PWP1, PWWP2A, PXK, PXN, PYDC2, QKI, RAB11FIP5, RAB35, RABEP1, RAC1, RAC2, RAD1, RAD17, RAD18, RAD21, RAD23A, RAD23B, RAD50, RAD51, RAD51B, RAD51C, RAD51D, RAD52, RAD54B, RAD54L, RAD9A, RAF1, RAG1, RAI14, RALGAPA1, RALGDS, RANBP17, RANBP2, RANBP3, RANGAP1, RAP1GDS1, RARA, RASA1, R131, RBBP8, RBFOX2, RBM10, RBM11, RBM15, RBMX, RCN1, RDM1, RECQL, RECQL4, RECQL5, REG1A, REG1B, REG3A, REG3G, REL, RELA, RELB, RERE, RERG, RET, REV1, REV3L, RFWD2, RGPD8, RGS18, RHEB, RHOA, RHOB, RHOH, RHOT1, RICTOR, RIF1, RIMS2, RIOK1, RIOK2, RIOK3, RIPK1, RIPK2, RIPK3, RIPK4, RIT1, RMI2, RNASEL, RNF10, RNF111, RNF144A, RNF168, RNF185, RNF213, RNF34, RNF4, RNF43, RNF8, RNGTT, ROBO3, ROCK1, ROCK2, ROR1, ROR2, ROS1, RP11-160N1.10, RP11-181C3.1, RP11-683L23.1, RP11-758M4.1, RPA1, RPA2, RPA3, RPA4, RPGR, RPL10, RPL1OL, RPL13A, RPL22, RPL5, RPN1, RPP38, RPS27, RPS6KA1, RPS6KA2, RPS6KA3, RPS6KA4, RPS6KA5, RPS6KA6, RPS6KB1, RPS6KB2, RPS6KC1, RPS6KL1, RPTOR, RQCD1, RRAD, RRAS, RRAS2, RRM1, RRM2B, RSPO2, RSPO3, RSRC1, RUNDC3B, RUNX1, RUNX1T1, RUNX2, RXRA, RYBP, RYK, RYR1, RYR2, SACM1L, SAMHD1, SATB2, SAV1, SBDS, SBF1, SBF2, SBK1, SBK2, SBK3, SCN5A, SCYL1, SCYL2, SCYL3, SDC4, SDHA, SDHAF2, SDHB, SDHC, SDHD, SEC23B, SEC31A, SECISBP2, SEMA3C, SEMA3E, SEMG1, SEPT5, SEPT6, SEPT9, SERPINB3, SERPINB4, SET, SETBP1, SETD2, SETDB1, SETDB2, SETMAR, SETX, SF3B1, SFPQ, SFRP1, SGK1, SGK2, SGK223, SGK3, SGK494, SGPP1, SGPP2, SH2B3, SH2D1A, SH3GL1, SH3PXD2A, SHFM1, SHE, SHOC2, SHPRH, SHQ1, SI, SIK1, SIK2, SIK3, SIN3A, SIRT1, SIRT2, SIRT3, SIRT4, SIRT5, SIRT6, SIRT7, SKI, SKP2, SLC12A2, SLC13A1, SLC17A8, SLC1A2, SLC22A13, SLC25A10, SLC25A4, SLC25A5, SLC26A3, SLC34A2, SLC38A4, SLC3A2, SLC45A3, SLC5A7, SLC9B1, SLCO1B1, SLIT2, SLITRK6, SLK, SLX1A, SLX1B, SLX4, SMAD2, SMAD3, SMAD4, SMARCA2, SMARCA4, SMARCAD1, SMARCB1, SMARCD1, SMARCE1, SMC1A, SMC3, SMC4, SMCHD1, SMG1, SMG7, SMO, SMUG1, SMYD4, SNAP91, SNCAIP, SND1, SNRK, SNTG2, SNX29, SNX31, SOCS1, SOS1, SOS2, SOX10, SOX17, SOX2, SOX9, SP2, SPAG16, SPANXN1, SPANXN2, SPATA6, SPECC1, SPEG, SPEN, SPHKAP, SPNS1, SP011, SPOCK3, SPOP, SPRED1, SPRR2G, SPRTN, SPRY1, SPRY2, SPRY4, SPTA1, SPTAN1, SPTBN1, SQSTM1, SRC, SRCAP, SRCIN1, SRGAP3, SRM, SRPK1, SRPK2, SRPK3, SRRM2, SRSF2, SRSF3, SS18, SS18L1, SSH1, SSH2, SSH3, SSX1, SSX2, SSX2IP, SSX4, STAG1, STAG2, STAG3, STARD6, STAT3, STAT4, STAT5B, STATE, STEAP4, STIL, STIP1, STK10, STK11, STK16, STK17A, STK17B, STK19, STK24, STK25, STK3, STK31, STK32A, STK32B, STK32C, STK33, STK35, STK36, STK38L, STK39, STK40, STRADA, STRADB, STRN, STYK1, STYX, STYXL1, SUFU, SULT1A1, SULT1B1, SUPT4H1, SUPT5H, SUZ12, SV2C, SVIL, SWI5, SYK, SYNE1, SYNJ1, SYNJ2, SYT4, TAB 1, TAC Cl, TADA1, TADA2B, TAF 1, TAF 15, TAF1A, TAF1L, TALL TANC2, TAOK1, TAOK2, TAOK3, TAS2R10, TAS2R13, TAS2R14, TAS2R43, TAS2R60, TBC1D2B, TBC1D31, TBCK, TBK1, TBL1XR1, TBP, TBX15, TBX22, TBX3, TCEA1, TCF12, TCF3, TCF4, TCF7, TCF7L2, TCL1A, TDG, TDP1, TDP2, TEC, TECRL, TEK, TENC1, TENM3, TERT, TESK1, TESK2, TET1, TET2, TEX13A, TEX14, TFDP1, TFE3, TFEB, TFG, TFPT, TFRC, TGFBR1, TGFBR2, TGIF1, TGIF2LX, TGOLN2, THADA, THEM5, THEMIS, THRAP3, TICAM1, TIE1, TIMM50, TJP2, TLK1, TLK2, TLR4, TLX1, TLX3, TMCO5A, TMED4, TMEM101, TMEM127, TMEM43, TMPRSS2, TMTC1, TNC, TNFAIP3, TNERSF10C, TNERSF11A, TNERSF13B, TNERSF14, TNERSF17, TNIK, TNK1, TNK2, TNKS, TNKS1BP1, TNKS2, TNNI3, TNNI3K, TNNT2, TNPO1, TNS1, TNS3, TOB2, TOM1, TOP1, TOP2A, TOP3A, TOPBP1, TP53, TP53BP1, TP53RK, TP53TG3D, TP63, TPM1, TPM3, TPM4, TPMT, TPR, TPSAB1, TPSB2, TPST1, TPTE, TPTE2, TRADD, TRAF2, TRAF3, TRAF7, TRAT1, TRDN, TREX1, TREX2, TRIM24, TRIM27, TRIM28, TRIM33, TRIM58, TRIM7, TRIML2, TRIO, TRIP11, TRMT10C, TRPM1, TRPM3, TRPM4, TRPM6, TRPM7, TRPV4, TRRAP, TSC1, TSC2, TSHR, TSHZ2, TSHZ3, TSPAN19, TSSK1B, TSSK2, TSSK3, TSSK4, TSSK6, TTBK1, TTBK2, TTK, TTL, TTN, TUBA1A, TUSC3, TWF1, TWF2, TXK, TXNIP, TYK2, TYMS, TYRO3, U2AF1, UBALD1, UBE2A, UBE2B, UBE2N, UBE2NL, UBE2V2, UBE2Z, UBE4A, UBLCP1, UBR5, UBXN11, UGT1A1, UGT1A7, UGT2A3, UGT2B28, UHMK1, UHRF1BP1L, ULK1, ULK2, ULK3, ULK4, UNG, UQCRFS1, USP2, USP28, USP29, USP6, USP7, USP9X, UTP14A, UTY, UVSSA, VAT1L, VCPIP1, VCX2, VEGFA, VEGFC, VEZE1, VEZT, VHL, VKORC1, VRK1, VRK2, VRK3, VTCN1, VTI1A, WAPAL, WAS, WBSCR17, WDR49, WDR52, WDR74, WEE1, WEE2, WHSC1, WHSC1L1, WIF1, WISP3, WNK1, WNK2, WNK3, WNK4, WNT2, WRN, WT1, WWTR1, XAB2,)(BPI, XIAP, XPA, XPC, XPO1, XPOT, XRCC1, XRCC2, XRCC3, XRCC4, XRCC5, XRCC6, YAP1, YARS, YES1, YME1L1, YPEL5, YWHAE, ZAP70, ZBBX, ZBTB16, ZBTB2, ZBTB7B, ZCCHC3, ZCCHC8, ZDHHC14, ZDHHC16, ZEB2, ZFHX3, ZFP36L1, ZFP36L2, ZFP41, ZIC4, ZMAT4, ZMYM2, ZMYM3, ZMYM4, ZMYND8, ZNF100, ZNF132, ZNF208, ZNF217, ZNF268, ZNF28, ZNF300, ZNF324, ZNF331, ZNF384, ZNF429, ZNF444, ZNF451, ZNF488, ZNF492, ZNF493, ZNF521, ZNF567, ZNF598, ZNF668, ZNF676, ZNF703, ZNF705G, ZNF708, ZNF716, ZNF717, ZNF727, ZNF750, ZNF799, ZNF80, ZNF804A, ZNF804B, ZNF812, ZNF814, ZNF844, ZNF91, ZNF98, ZNF99, ZNRF3, ZPBP, ZRSR2, ZSWIM2, MYCL, MYCL, MLK4, MLK4, ZAK, FRG1B, FRG1B, TRBV5-4.

The biomarkers may be selected from one or more intron source including: ALK, BRAF, BRD3, BRD4, EGFR, ERG, ETV1, ETV4, ETV5, EWSR1, FGFR1, FGFR2, FGFR3, MET, NOTCH1, NRG1, NTRK1, NTRK2, NTRK3, NUTM1, PDGFRA, PDGFRB, PRKCA, PRKCB, RAF1, RET, ROS1, TMPRS S2.

The biomarkers may be selected from one or more promoters including: AC099552.4, ADAMTS10, AGBL4, ANKRD3OBL, ANKRD53, AP003733.1, AP2A1, ARHGEF18, ARHGEF35, BCL2, BCL2L11, C16orf59, C4orf27, CABLES2, CACNA1C, CBWD1, CCDC107, CDC20, CDH18, CHMP3, COL11A1, CYLD, CYP4F2, DI02, DLG2, DNAJA2, EZH2, FAM129C, FAM21A, FCGR3B, GALNT13, GOLGA2, GPR89A, GTF2I, GTF3C5, HCN1, HERC2, HKR1, IGFBP7, INSR, ISOC2, ITPR1, KALRN, KLRG1, LENG9, LEPROTL1, LTV1, LUC7L2, MAGEA3, MASTL, MED16, MEF2C, MGRN1, MPND, MRPS9, MTRNR2L1, MTRNR2L8, MYNN, MYOZ3, NALCN, NCOA7, NEK11, NFKBIE, NPAS3, NPEPPS, NXPE1, OR2L2, OR2W3, OR9G1, OXNAD1, PACS1, PADI4, PAPD5, PFN2, PLEKHS1, POLR2D, POU5F1B, PPAPDC1A, PRSS1, RAI14, RGPD8, RNF185, RNF34, RPL13A, RPS27, SECISBP2, SLC12A2, SMG1, SMUG1, SNTG2, SP2, STAG3, STAG3L5P-PVRIG2P-PILRB, TBC1D2B, TBC1D31, TCF3, TCL1A, TERT, TNK2, TPM3, TPSAB1, TPSB2, TPTE, TRBV5-4, TRMT10C, TRPM4, TRPV4, VCPIP1, WDR74, ZDHHC16, ZNF324, ZNF488, ZNF708, ZNF716, ZNF717, ZNF727, ZNF799.

The biomarkers may be selected from the microsatellite instability (MSI) source including ADGRG6, ALG10B, BAT25, BAT26, BCL11B, BCL2, BCL6, BCL7A, Clorf159, CALM1, CTNNA2, D175250, D2S123, D5S346, DHX16, DLX4, DRD5, EEF1A1, FGF7, FLI1, FSCN3, GNAS, GP6, HPCAL4, INPP4B, LRRC4C, MAP2K2, MAT2A, METRNL, NR21, NR22, NR27, PES1, PLCL1, PRELID2, RCN1, TBC1D31, TENM3, TOB2, TP53TG3D,) (BPI, ZFP41, ZNF208.

The biomarkers may be selected from viral genomes that are known to be involved in cancer including human papillomavirus (HPV), Herpes Simplex (HSV), Epstein-Barr Virus (EBV), Hepatitis B Virus (HBV), Hepatitis C Virus (HCV), Human T-lymphotropic Virus 1 (HTLV-1), Human Herpesvirus-8 (HHV8). A genetic variant or alteration may be a single nucleotide variant, an indel, a transversion, a translocation, an inversion, a deletion, a chromosomal structure alteration, a gene fusion, a chromosome fusion, a gene truncation, a gene amplification, a gene duplication and a chromosomal lesion.

### Therapy Matching

In another aspect, the present disclosure provides a computer-implemented method for providing a subject displaying cancer with a therapy. Biologic data may be received for a subject. The biological data may be generated from one or more biological samples of the subject. The biologic data can be used to generate a first list of therapies according to a molecular profile of the subject. The molecular profile may be indicative of one or more genomic aberrations in one or more biological samples. A second list of therapies may be generated from a first list of therapies using medical history data of the subject. The list of therapies may comprise clinical trial(s) and/or standard of care. The second list of therapies may be presented to a subject on a user interface. The second list of therapies can be presented to a clinician to select for a recommended therapy. The subject may also receive a request for enrollment in a given therapy from the second list of therapies.

During acquisition of biological data, the biological data may be generated from one or more biological samples of the subject. The biologic data may be generated from one or more biological samples of the subject without any pipetting by a user during preparation of one or more biological samples. Alternatively, the biologic data may be generated from one or more biological samples of the subject with pipetting by a user during preparation of one or more biological samples. The biologic data may comprise data generated from one or more biological samples selected from the group consisting of protein, peptides, cell-free nucleic acids, ribonucleic acids, deoxyribose nucleic acids, and any combination thereof. The biologic data may comprise a molecular profile that is indicative of one or more genomic aberrations in one or more biological samples. One or more genomic aberrations can include nucleic acid mutations and/or differentially expressed proteins. Nucleic acid mutations may be selected from the group consisting of an insertion(s), nucleotide deletion(s), nucleotide substitution(s), amino acid insertion(s), amino acid deletion(s), amino acid substitution(s), gene fusion(s), copy-number variation(s), and genes or variants selected from Table 1.

A panel of molecular assays may be used for DNA, RNA, and protein analysis. The tumor tissue DNA assay may be a highly sensitive, next generation sequencing (NGS) based somatic mutation detection across at least about 100, at least about 500, at least about 1000, at least about 1500, at least about 2000, at least about 2500, at least about 3000, or at least about 4000 genes or at least about 20, at least about 30, at least about 40, at least about 50, at least about 60, at least about 70, at least about 80, at least about 90, at least about 100, at least about 150, at least about 200, at least about 250,or at least about 300 introns. The tumor tissue DNA assay may meet the analytical standards for Medicare coverage. The circulating tumor DNA (ctDNA) assay may be a non-invasive, liquid biopsy of circulating tumor DNA. Additionally NGS based mutation detection may be obtained for at least about 100, at least about 200, at least about 300, at least about 400, at least about 500, at least about 600, at least about 700, at least about 800, at least about 900, at least about 1000, at least about 1500, or at least about 2000 genes. The tumor RNA-sequencing assay may be NGS-based, whole transcriptome sequencing. The tumor IHC assay may be an immunohistochemical testing of key oncology proteins and immune-oncology markers.

The biologic data can be used to generate a first list of therapies according to a molecular profile of the subject. Alternatively, the subject's medical history data and biologic data may be used concurrently to generate the first list of therapies. Generating a first list of therapies may comprise querying one or more databases for one or more targeted therapies according to a predetermined gene or genomic region. Matches with therapies according to molecular requirements may be grouped based on matching specificity to the subject's molecular profile. For example, therapies that match for a specific point mutation can be grouped in separate category than therapies that match for mutations of a gene. Therapy databases can comprise public repositories or trials obtained from specific affiliations. Public repositories can include a database selected from the group consisting of ClinicalTrials.gov, National Institute of Health, Research Match, and national registries, such as the breast cancer family registry and the colon cancer family registry. Trials obtained from a specific affiliation can comprise knowledge of trials that are not accessible in a public repository and can be obtained from an affiliated institution.

The first list of therapies may exclude therapies that target genomic aberrations absent in one or more biological samples. Generating a first list of therapies can also comprise removing therapies that target genomic aberrations absent in one or more biological samples. Generating a first list of therapies (e.g. clinical trials) can also comprise sorting the therapies into two categories. The two categories may include therapies that target the subject's mutation and therapies that do not specify a molecular target. Matches of the therapies according to molecular requirements may be determined based on matching specificity to the subject. For example, therapies that match for a specific point mutation can be differentiated from therapies that match for mutations of a gene. The therapies may be matched to a subject according to labels identifying the profile of the subject. The labels may be questions targeted to understanding the subjects's molecular and medical history and status. Labels can be generated according to a topic selected from the subject's genomic and biomarker profile, diagnosis status, prior therapies conducted on the subject, outcomes of prior therapies conducted on the subject, and other comorbidities.

The first list of therapies may additionally be filtered according to phases of the therapy. For example, phases of a therapy may be phases of a clinical trial. Clinical trials can comprise five phases: phase 0, phase 1, phase 2, phase 3, and phase 4. Phase 0 may comprise human micro dosing studies. Data from phase 0 can accelerate the development of promising drugs or imaging agents by determining early on whether a drug or agent can behave in human subjects as was expected from pre-clinical studies. Phase 1 may be the first-in-man studies and can be the first stage to test the drug in human subjects. In phase 1, the maximum dosage of a drug administered to a subject before adverse effects become dangerous or intolerable can be determined. This group of clinical trials may be operated by the contract research organization (CROs). During phase 2, the drug can be tested for biological activity or effect. A group of at least about 50, at least about 100, at least about 150, at least about 200, at least about 250, at least about 300, at least about 350, or at least about 400 subjects can be enrolled during the phase 2 studies. During phase 3, the effectiveness of the new drug may be determined and the value of the new intervention can be assessed. A group of at least about 100, at least about 150, at least about 200, at least about 250, at least about 300, at least about 350, at least about 400, at least about 500, at least about 1000, at least about 2000, and at least about 3000 subjects can be enrolled during the phase 3 studies. Phase 4 trials may comprise determining safety surveillance and ongoing technical support of a drug after it has been approved for sale.

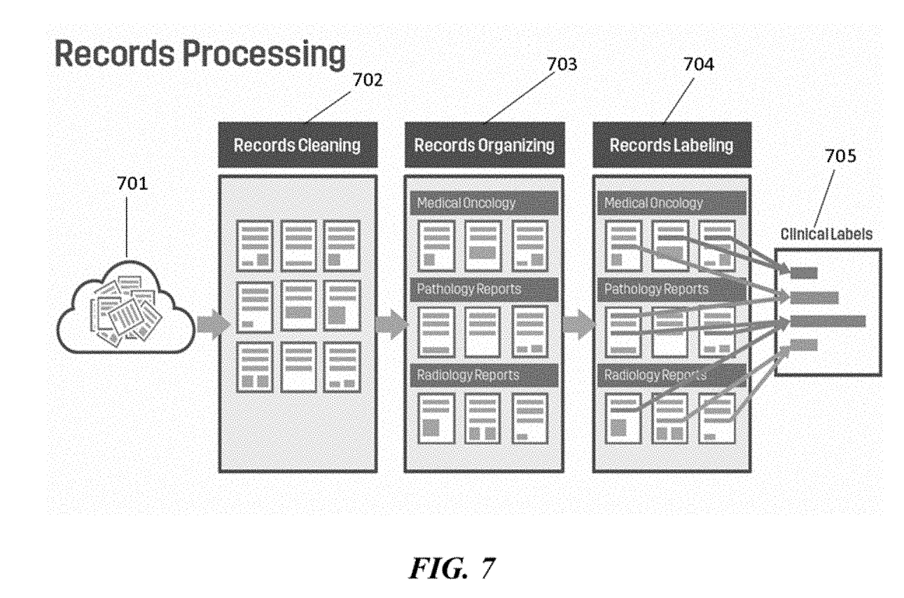

A second list of therapies may be generated from a first list of therapies using medical history data of the subject. Alternatively, the subject's medical history data and biologic data may be used concurrently to generate the first list of therapies. The second list of therapies may be the first list of therapy. Medical history data for a subject may be received and processed according to FIG. 7 to determine a subject's current health state and qualification for a targeted clinical trial matched from the subject's biologic data. The medical history data 701 may comprise information selected from the group consisting of identification, demographics, history of present illness, past medical history, review of systems, family diseases, childhood diseases, social history, regular and acute medications, allergies, sexual history, obstetric and gynecological history, surgical history, medication, habits, immunization history, growth chart and developmental history. The review of systems may comprise cardiovascular system, respiratory system, gastrointestinal system, genitourinary system, nervous system, cranial nerves symptoms, endocrine system, musculoskeletal system, and skin. The medical history data may be processed and can prevent social desirability bias. The processing method may be selected from the group consisting of cleaning 702, organizing 703, and labeling 704 the subject's medical history to generate a processed set of clinical records with the relevant labeled medical text segments 705. Prior to medical records data processing, the medical record may be requested and then submitted for retrieval. Proper authorization to collect the records may be obtained. The authorization request can be in the form of an automatically generated fax, mail, e-mail, or utilize the Internet to deliver the requested records to the system. Once collected, the medical records may be received or converted to an electronic or digital file format, for efficient processing. The medical records may be checked for quality by examining quality features, such as legibility, completeness, and accuracy. Components of the system can be trained to recognize document types and to check quality on each page of the documents. After the quality check, the medical records can be prepared for abstraction. Abstraction may be the analysis conducted by the abstractor of the received records to look for specific information requested by the client, including specific services for the patient (such as lab tests, prescriptions, screening tests, etc.) or all services provided. Abstraction may be conducted manually or automatically. Manual abstractors can have a wide range of qualifications and backgrounds, and can include registered nurses (RN), licensed vocational nurses (LVN), licensed practical nurses (LPN), certified coders, registered health information administrators (RHIA), registered health information technicians (RHIT). Following abstraction, an overread process can check for the quality of the analysis or abstraction conducted by the abstractors to assure accuracy and completeness. Once processed, the designated, specified, or authorized medical records or documents may be securely accessed through a portal website by a subject.

The medical history data may also be labeled according to relevant medical text segments. The medical history data may be processed into the label name, the label category, and the label value. The label name indicates a question identifying one or more relevant portions of the medical history data. The label category may be a grouping and/or classification of one or more label names. The label value may be an answer to the label name. The label value may be selected from the group consisting of yes, maybe, and no. The label value may correspond to the group consisting of yes, maybe, and no. A medical text segment may be a word or phrase in a medical record that can be used to confirm an eligibility requirement for a clinical trial. There can be an abundance of text in medical records but only a small subset of it is relevant to determine the eligibility of a subject for a trial. The medical text segment may comprise a proprietary set of topics. Labeling can comprise extracting from the first list of therapies a second list of therapies. The labels can comprise questions targeted to understanding the subject's profile, prior therapy history and outcomes from prior therapies. Labeling can be accomplished manually or automatically. Manual labeling can involve a lengthy review of patient records and trial criteria descriptions. The machine learning model can detect and label the relevant medical text segments. Different weight may be assigned to different subject parameters depending on the particular medical condition being treated and on the particular patient being treated. Machine learning prediction can be used to generate vectors to calculate similarity and to generate a set of scores for matching between the subject's clinical trial eligibility and the medical records.

The subject's clinical trial eligibility that is pre-filtered by the subject's molecular profile may be combined with a subject's medical records into a natural language processor (NLP). State of the art NLP and information extraction (IE) techniques may be customized and implemented to build the automated eligibility screening (ES) architecture. Eligibility criteria can include a demographics filter such as a filter for age, race, geographic data, physical data, financial data, and gender. A trial enrollment window may also be used to expedite a pre-filtering process. For example, if a subject did not have clinical data within a start date and closing date of an enrollment window of at least, the subject may be removed from participating in a specific clinical trial. Text and medical terms processing can utilize advanced NLP methods to extract medically relevant information from the patient medical history records. During NLP extraction, an algorithm may be generated to first extract medical information using acronyms and keywords from an extraction system. The extraction system may be a custom designed extraction system. The extraction system may be the Apache clinical Text Analysis and Knowledge Extraction System (cTAKES). Extraction systems, such as cTAKES, can assign medical terms to the identified text strings from controlled terminologies such as Concept Unique Identifiers (CUI) from the Universal Medical Language System (UMLS), standardized nomenclature for clinical drugs (RxNorm), and Systematized Nomenclature of Medical Clinical Terms codes (SNOMED-CT). This process can also be utilized for identifying medical terms and texts from the diagnosis strings. Additionally, codes from the international classification of diseases, such as ICD-9 codes, can be mapped to SNOMED-CT terms using the UMLS ICD-9 to SNOMED-CT dictionary. A negation detector can also be utilized to determine negations. The negation detector may be based on the NegEx algorithm. Identified medical terms and texts can be stored as a bucket of words in a subject vector. Such an inclusion exclusion technique can be derived from medical terms and text processing to pull term-level patterns. All terms pulled from the exclusion criteria can be transformed into the negated format. The medical terms and texts extracted from a subject's Electronic Health Record (EHR) can be stored in a vector that is a representation of the subject's profile. The Bayesian network may be used to infer the marginal probability of label values given other labels' values observed in a subject's medical records as well as from aggregated population data. Bayesian Networks may be used to infer medical history that is not explicitly found in the subject's medical records. Bayesian networks may be used to infer labels or label values not found in the medical text but using relationships between labels that are found in the text and/or informed by population-level data. Alternatively, statistical learning algorithms may be used to infer aspects of the medical history not available in the text based on population data.

Generation of the first or second list of therapies can also comprise determining ineligible therapies according to a categorical score and rejecting ineligible therapies from remaining therapies to generate a filtered list of remaining therapies. The categorical score can be selected from the group consisting of yes, maybe, and no. The categorical score may correspond to the group consisting of yes, maybe, and no. Boolean logic may be used to calculate whether any given label's value as assessed for a subject by the system is a mismatch with the expected label values in the criteria crucial to therapy enrollment. If a subject's value for a given label is mismatched with the expected value for a given label, as expressed in the criteria for a therapy, then the subject maybe ineligible for the therapy. The therapies may be grouped using a similarity score between the subject and all the therapies based on the labels. One similarity metric used can be finding an empirical significance threshold and determining positive therapies by a specific criterion and then assessing overlap among positive therapies in a standard manner. Contrarily, a dissimilarity measure can be a numerical measure of the degree to which two objects are different. The therapies that fall below a minimum similarity score for criteria crucial to therapy enrollment can be ineligible. The list of remaining therapies may then be compared and reviewed. The review may generate a first list or second list of therapies.

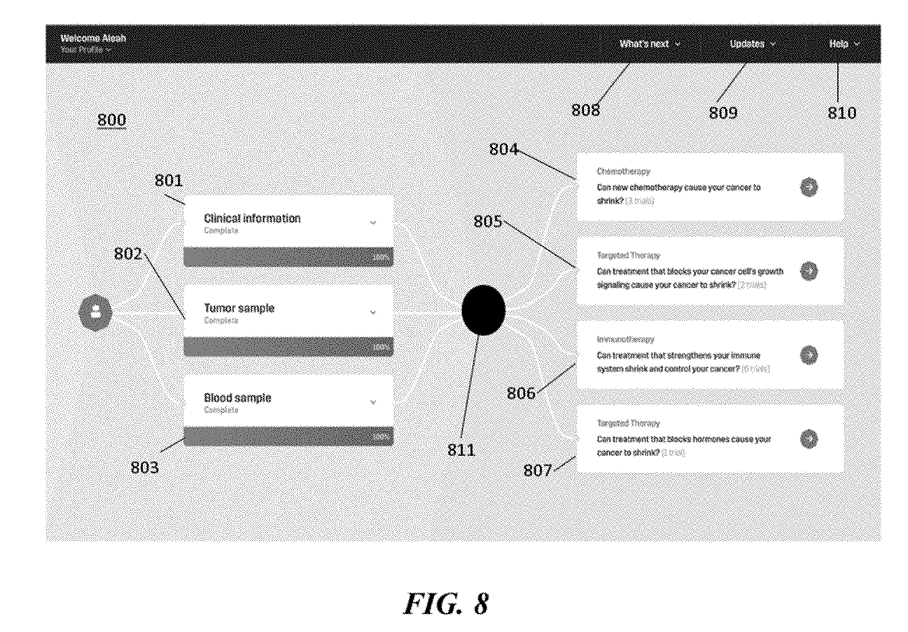

The first list or second list of therapies may be passed to a user to manually verify eligibility using links to information from the medical history data and the biologic data for the subject. The user may be a healthcare professional or a primary care provider of the subject. The therapy filtering preferences can be selected from the group consisting of availability at a specific institution, availability at a set of institutions, type of treatment, phase of clinical trial, method of drug delivery, location and distance of a given therapy from a specified location, duration of treatment, and patient relocation therapy duration. The types of treatment may be selected from the group consisting of immunotherapy, targeted therapy, chemotherapy, radiation therapy, hormone therapy, stem cell transplant, precision medicine, and surgery. Methods of drug delivery can comprise non-invasive peroral, topical, transmucosal, and inhalation routes. Transmucosal route can comprise nasal, buccal/sublingual, vaginal, ocular and rectal. Filtering can further comprise an evaluation by a healthcare professional and a selection for a recommended therapy. A group of at most 10, 15, 20, 25, 30, 35, 40, 45, or 50 therapies may be presented to a clinician to select for a recommended therapy. The therapies may then be passed for a final authorization by a medically qualified staff member to review therapies based on the proprietary labels, and using their expert knowledge rule out groups of labels that are less successful for the subject. The subject may access a link to the matched therapies on their profile webpage on the user interface. The subject may receive an email with a link to the matched therapies. The matched therapies may be displayed on a user interface. The user interface may display the status of the acquisition of medical history data and biologics data. The user interface may display matched therapies organized according to categories such as chemotherapies, targeted therapies, immunotherapies, and radiotherapies. FIG. 8 shows an example profile 800 of a subject after the completion of treatment matching 811. The profile indicates the status of the acquisition of the clinical information 801, tumor sample analysis 802, and blood sample analysis 803. The clinical information may be the medical history data. The medical history data may be the medical records. The profile may also display links to the categorized therapies, for example, the chemotherapy category 804 has three clinical trials directed to the question “can new chemotherapies cause your cancer to shrink?” and the targeted therapy category 807 has one clinical trial directed to the question “can treatment that blocks hormones cause your cancer to shrink?”. Similarly, the question along with the matched clinical trials may be displayed other targeted therapy categories 805 and for immunotherapy categories 806. A tab for next steps 808, updates 809, and help 810 may be accessed through the subject's profile.

A subject may then receive a request for enrollment in a therapy through a user interface. A selection from the subject may be received as to one or more therapies. A request for enrollment may be received from the subject in a therapy selected from the therapies through the user interface. Any therapy can be added to a subject profile for a subject. A caregiver may view all profiled therapies of the subject. If desired, a new clinical trial can be profiled. The name of a new clinical trial can be entered into the subject's therapy system. As part of the subject's profile, the subject may select for a crowd funding option to aid in the cost of his or her cancer therapy. The crowd funding option may connect the subject to links such as YouCaring.com, FundRazr, GoFundMe, GiveForward and Indiegogo.

### Clinical Trial and Medical History Outputs

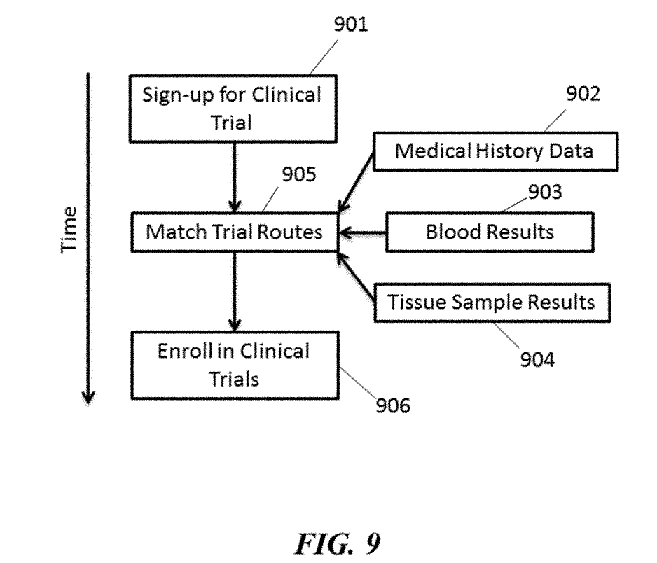

In another aspect, the present disclosure provides a computer-implemented method for qualifying a subject for a clinical trial FIG. 9. The subject may sign-up for a clinical trial 601. Medical history data and biologic data may be received for the subject 902, 903, and 904. The biologic data may be automatically generated from one or more biological samples of the subject without any involvement of a user. One or more databases for one or more clinical trials corresponding to the medical history data and the biologic data may be queried to generate a set of clinical trials for which the subject qualifies 905. The set of clinical trials may comprise at least one clinical trial. A set of clinical trials may be provided on a user interface for display to a user. A request for enrollment of the subject in a clinical trial selected from the provided set of clinical trials may be received through the user interface 906. The request may be received over a network. The curated clinical trials may be a combination of clinical trials. Enrollment of the subject may be determined by eligibility of the subject and efficacy of the subject's response to the clinical trial. Enrollment may be achieved by a combination of end-to-end patient engagement followed by leveraging insights from therapeutics research for guidance on recommended trials.

In another aspect, the present disclosure provides a method for qualifying a subject for a subset of therapies. The medical history data and biologic data may be received for the subject. The biologic data may be generated from one or more biological samples of the subject. The medical history data and the biologic data may be analyzed to yield a genomic-based medical history analysis for the subject. The genomic-based medical history analysis may be used to query one or more databases of therapies for the subject and to generate the subset of therapies for which the subject qualifies. Then, the subset of therapies can be presented on a user interface on an electronic device of a user.

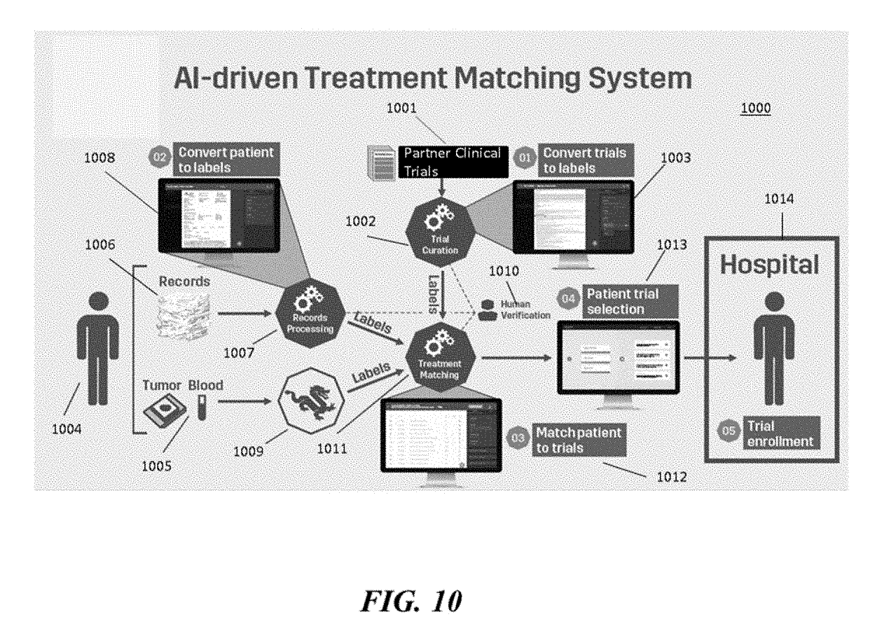

FIG. 10 illustrates the treatment matching system 1000 using a data base of therapies (e.g. clinical trials) 1001, the subject's biological sample 1005, and the subject's medical records 1006. A database of therapies 1001 may be assessed against one or more criteria for eligibility during trial curation 1002. Eligibility criteria can be selected from the group consisting of age, race, gender, geographic data, physical data, financial data, medical history, a particular type of cancer, a particular stage of cancer, and current health status. The computer assessment may include identifying at least one portion of the database of therapies according to the eligibility criteria. The data base of trials may be analyzed to generate a filtered list of therapies 1003. Concurrently or separately, the biological sample 1005 and the medical history records 1006 may be obtained from the subject 1004. The biological sample 1005 and the medical history records 1006 may be processed and labeled according to the methods disclosed herein 1007 and 1009 respectively. The labeled subject records 1008 and the labeled biologic data can then query the filtered list of therapies 1003 to generate a matched subset of therapies for which the subject qualifies 1012. The matched therapies may be presented on a user interface for the subject to view 1013. The subject can select for one or more trials and submit a request for enrollment 1014. Additionally, human validation 1010 may be performed on the trial curation process 1002 and the records processing 1007.

During therapy curation 1002, an abundance of therapy criteria may be condensed using a set of labels as identifiers of relevant portions of the therapy data. For example, trial 1 may require the subject to be absent of lesions in the brain, trial 2 may require the subject to be free of central nervous system involvement, and trial 3 may require the subject to be absent of leptomeningeal disease. The label for these three requirements may be identified as “Does the patient have brain metastases?” and the required answer would be “No” if the subject is to qualify for the three therapies. The required answer may be obtained by reviewing the subject's biologic data and medical history data.

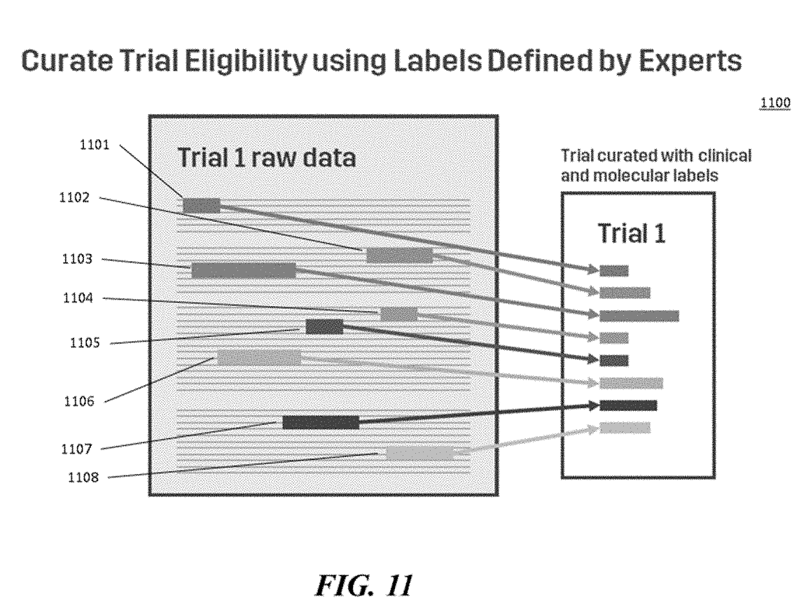

FIG. 11 shows a clinical trial curation process 1100 according to eligibility criteria with one or more of labels. The entire set of data 1109 from a therapy may be obtained and processed to identifying relevant portions of data 1101-1108 from the full set of data. The relevant portions are then extracted and summarized into a condensed data sheet for the therapy 1110. The therapy 1110 may be curated with clinical and molecular labels.

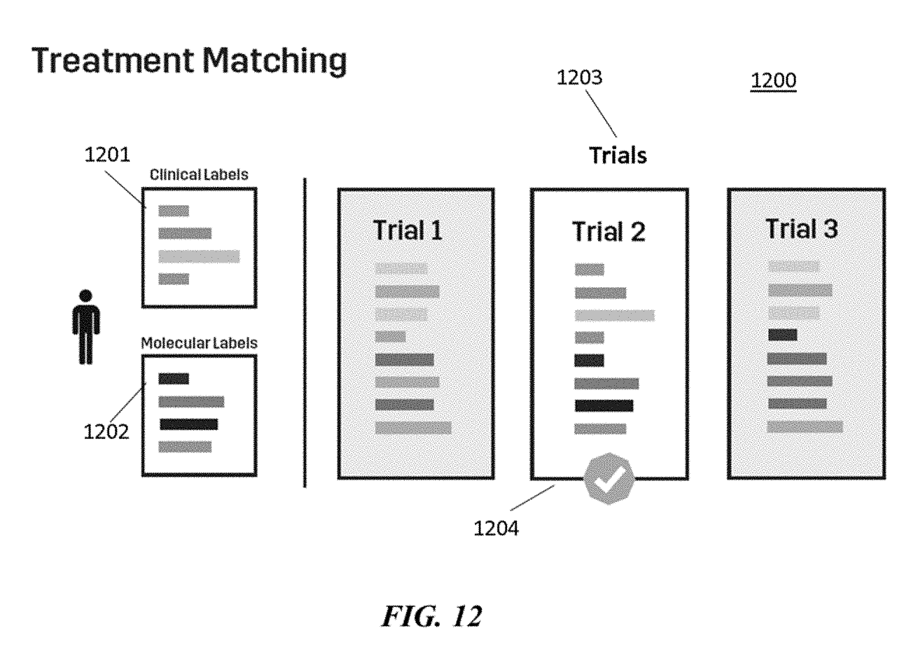

In the treatment matching 1200 of FIG. 12, the medical history record labels 1201 and the biologic data labels 1202 may be matched against the filtered list of therapies 1203 to identify one or more therapies 1204 comprising the labels identified in the subject's medical history record and biologic data.

A software based laboratory and management system may be utilized. The system may be a laboratory information management system (LIMS). The LIMS may comprise features that support a modern laboratory's operations.

The biologic data from the one or more biological samples of the subject may be automatically generated without any involvement of the user. The biological data may be used for cloud based clinical trial matching, clinical trial enrollment, treatment matching, records acquisition, and drug development. One or more clinical trials within the generated set of clinical trials may be prioritized. The prioritizing may be based on one or more factors selected from the group consisting of: geographic location of the clinical trial, regulatory approval status, annotated medical history data for the subject, or a combination thereof.

In another aspect, the subject may qualify for one or more therapies. The method may include receiving a first nucleic acid sample from a tumor tissue sample of the subject and a second nucleic acid sample from a normal tissue sample of the subject. The first nucleic acid sample and second nucleic acid sample may be obtained from the tumor tissue sample and the normal tissue sample automatically without any involvement from a user. Next, the first nucleic acid sample and second nucleic acid sample may be assayed to identify one or more genomic alterations in the tumor tissue sample relative to the normal tissue sample to generate a set of genomic data for the subject. The databases may be queried for one or more therapies (e.g. clinical trials) corresponding to a medical history of the subject and the genomic data to generate a set of therapies. The therapy may comprise at least one therapy that has a predicted likelihood of success that is at least about 90%. A set of therapies and standard treatment options, such as treatment options based on National Comprehensive Cancer Network (NCCN) guidelines, may be presented on a user interface for display to a user.

In preparation for a therapy, subjects may be recruited. Several factors may be considered in qualifying a subject for a therapy or enrolling a subject in a therapy. Factors considered may include geographical feasibility or location, population research, optimal recruiting site selection, site assessment, recruitment materials, media support, media management, site training materials, study website, patient referral follow-up, translations, community outreach, physician outreach, site support, and monitoring and reporting for assessment of patient recruiting activities. For subjects participating in global clinical studies, patient retention services may be a factor. The subject retention services can include visit reminders, patient support items, and care giver support.

During enrollment of a subject into therapies, the database may be queried for one or more therapies corresponding to a medical history of the subject and genomic data to generate a set of therapies. Eligibility criteria can be another decisive factor for the types of clinical trial enrollment. Eligibility criteria may comprise age, gender, medical history, and current health status. For example, subjects may need to have a particular type and stage of cancer to participate in a particular trial. The subject may be comprise one or more of individual, a group of individuals, a medical professional providers including clinicians, physicians, dentists, nurse practitioners, radiologists, anesthesiologist, psychologists, pharmacist, psychiatrists, dental hygienists, nurses, dentists, chiropractors, physical therapists, occupational therapists, speech pathologists, nutritionists, orthodontists, laboratory personnel, medical coders, diagnostic center personnel, emergency\ambulatory medical personnel, a hospital, a health care providing organization, an HMO, an insurance provider, a government agency, or a financial institution, business entity (e.g., insurance company, employer, pharmaceutical company, academic institution, non-governmental organization, Medicare/Medicaid, or community health care provider.

The subject enrolled in the therapy may be monitored by assaying one or more biological samples from the subject. The assaying may be directed to at least about 50 genes, 100 genes, 200 genes, 300 genes, 400 genes, 500 genes, 1000 genes, 1500 genes, 2000 genes, or 2500 genes selected from Table 1. The likelihood of success for the subject may be predicted. One or more therapies may be annotated. Querying of one or more databases has a predicted likelihood of matching to a therapy of at least about 70%, 75%, 80%, 85%, 90%, or 95%.

Medical history may be retrieved for the subject. The medical history data may be automatically annotated in standardized terminology. The standardized terminology may be Unified Medical Language System. The medical history data may be inputted into the records acquisition and processing system and a resultant annotated medical history may be attained. The medical history may be editable file or non-editable files. Editable files may comprise one or more of medical history nutrition, habits, exercise regimen, medication, race, height, weight, demographics, event log, allergies, testing results, diagnostics electronic living will, DNA profile, DNA samples or markers, blood pressure ranges, blood sugar levels, mental health information, cancer treatment history, response to treatment, surgical interventions, history of present illness, review of organ systems, family and childhood diseases, regular and acute medications, sexual history, obstetric/gynecological history, health care encounters to include diagnosis and/or procedures or personal information contact information, address, work and occupation information, health savings account information, bank account information, authorized associate account information. Non-editable files can include but are not limited to a DNA profile, medication history, lab reports/results, digital images, binary attachment files, research data or a combination thereof. The file may be an immunohistochemistry report. The report may be a supplemental research report. The supplemental research report may be publications found based on genetic data. The medical history may also involve assessment of the cardiovascular system, respiratory system, gastrointestinal system, genitourinary system, nervous system, cranial nerves symptoms, endocrine system, musculoskeletal system, and the skin.

The medical history may be a personal health record. A personal health record can be content files. Examples of content files comprise past patient medical history, including treatment, illnesses, family history, past and current medications, and other content information, such as medical history. Other examples include X-rays, CT scans, MRI scans, blood screens/test results, medical treatment information, medical conditions (e.g., current, past, pre-existing), allergies to medications, current medications or any other results, laboratory results/reports, digital images, binary attachments (e.g., PDF files), research data, DNA profile or genome information, test, screens, and scans. The medical history content can be regularly updated. During a request for enrollment, the enrollment may be received over a network comprising one or more of an internet connection, a web browser, a portable communication device, a computer, a television, a telephone, ATM, network appliance or router. The user interface may be a web-based user interface.

Certain therapies may be prioritized within a generated set of clinical trials. Factors that affect the priority choice may include geographic location, regulatory approval status, and annotated medical history data.

The medical history of a subject may be requested by the subject. The medical history may be disparate. The documents can be inputted into the platform records acquisition and processing system and organized. The data may be used in determining outcomes of therapies. The data may also be used to examine the effects of tested drugs on subjects (e.g., patients) by studying the various outcomes of effects among different populations. During the examination, the therapy may be known. The therapy may also be unknown and the sample analysis platform (e.g., automated platform) may be used to generate a therapy for the subject. The data may be used in identifying the population of people that responded positively to the therapy and the common characteristics of the population. From the data, sequence and mutation targets may be identified and matched with a drug that affects the targets. As a result, a searchable database of drugs may be assembled. Patients may be directly connected with treatments. Existing treatments that the data may identify a match can lead to unanticipated effects. The unanticipated effects may be useful in the process of drug discovery.

During drug matching, a specific mutation may be identified in a sample and matched with a corresponding drug. The system may recommend a drug that can be useful in other similar pathways. The drug may be a drug approved by a government unit (e.g., Food and Drug Administration, FDA). The drug recommendation may be based on prior clinical history.

The medical history may be obtained from a doctor or patient database. The doctor database may comprise practice areas of the doctor or hospital, the number of patients in their practice, or the location of their practice. The patient database may comprise information regarding all the patients associated with a particular medical practice and can include their specific height, weight, age, gender, medical history, current health status or any particular genetic markers.

Furthermore, the database may include key words associated with the subject's medical history including dictations prepared by the medical professional; lab, radiology and pathological reports; blood work panels and other appropriate information. The database component can also include medical fees associated with relatively standard procedures that are performed by the medical professional such as blood tests, office visits, taking of vital signs, supervising and preparing a specific type of medical history, or performing a medical physical. The medical history may be described in standardized terminology. The standard terminology may be Unified Medical Language System. The user interface may be a web-based user interface or a mobile user interface.

In another aspect, the present disclosure provides a method qualifying a subject for enrollment in a therapy. A first nucleic acid sample from a tumor tissue sample of the subject and a second nucleic acid sample from a normal tissue sample of the subject may be received. The first nucleic acid sample and second nucleic acid sample can be obtained from the tumor tissue sample and the normal tissue sample automatically without any involvement from a user. Next, the first nucleic acid sample and the second nucleic acid sample may be assayed to identify one or more genomic alterations in the tumor tissue sample relative to the normal tissue sample to generate a set of genomic data for the subject. One or more databases for one or more therapies corresponding to a medical history of the subject may be queried. Curated databases of therapies and standards of care may be generated. The genomic data may be queried to generate a set of therapies for which the subject qualifies. A set of therapies on a user interface for display to a user may be provided. The method can also comprise receiving medical history data from the subject and a request for enrollment of the subject in a therapy selected from the provided set of therapies through the user interface. A therapeutic target based on the medical history and the genomic data may be identified. The subject may be enrolled into a therapies based on the identified target. The subject may be monitored. The monitoring can comprise assaying one or more nucleic acid samples to generate genomic data. The assaying may be directed to at least about 50 genes, 100 genes, 200 genes, 300 genes, 400 genes, 500 genes, 1000 genes, 1500 genes, 2000 genes, 2500 genes, or 2800 genes selected from Table 1. Assaying may comprise sequencing the first nucleic acid sample and the second nucleic acid sample without any involvement from a user. Assaying may further comprise receiving a request from the user to sequence the biological sample. The request can be received from the user to sequence the first nucleic acid sample and the second nucleic acid sample.

### Computer Control Systems

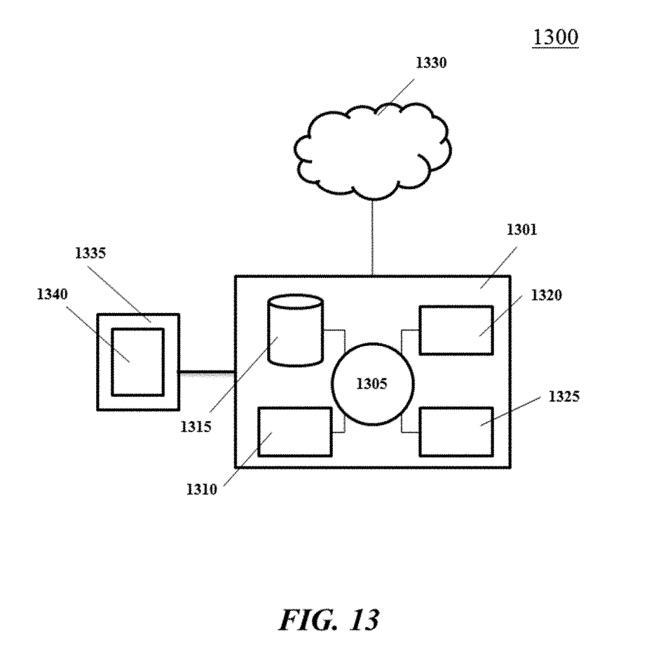

The present disclosure provides computer control systems that are programmed to implement methods of the disclosure. FIG. 13 shows a computer system 1301 that is programmed or otherwise configured to implement the methods of the present disclosure. The computer system 1301 can regulate various aspects sample preparation, sequencing and/or analysis, cloud based clinical trial matching, clinical trial enrollment, treatment matching, records acquisition and processing, and drug development. In some examples, the computer system 1301 is configured to perform sample preparation and sample analysis, including nucleic acid sequencing. The computer system 1301 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 710, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 13, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., an operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine have a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340. The UI can allow a user to set various conditions for the methods described herein, for example, PCR or sequencing conditions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, process the reads to generate a consequence sequence.

### EXAMPLES

The examples below are illustrative and non-limiting.

### Example 1

The Pre-Amplification Sample Processing is associated with sequencing preparations. The system operates on 5 iterations during a 10 hour work day. During each work day, 5 PCR plates are transferred to Post-Amplification System. During the Pre-Amplification sample processing, the lysis method is run on the liquid handler (Hamilton Star) with a deep well plate. A tip box is sent to waste. The plate is sealed and incubated for 30 minutes with shaking. Then the plate undergoes centrifugation for 2 minutes. The plate can then be peeled. The beads are added onto the liquid handler and loaded onto the DNA and extraction prep shelves (Kingfisher). The extraction protocol is run and comprises an additional wash and extraction of plates onto the Kingfisher. The QC plates on the fragment analyzer are read. If the samples are not suitable for further processing, the extraction protocol can be re-run. The destination tube rack may be placed on the docking table (Star). The data from the fragment analyzer is used to make the normalization plate on the Star. The sample may be aliquoted to the tube rack, re-capped, and sent to the output rack. During shearing, enzyme is dispensed to the normalized plate. The normalized plate is sealed and incubated with shaking for 1 hour. The plate is spun and the seal peeled. The QC end repair method is run on the Star. The plate on the fragment analyzer is read for QC. The normalized plate may be sealed and incubated with shaking for 1 hour. The normalized plate undergoes centrifugation and is then peeled. During adaptor ligation, the method is run on the Star and beads are added. The plate is moved to the Kingfisher and undergoes an additional wash and cleanup and eluent step. The magbead cleanup process is run on the Kingfisher. The remaining plates are removed to the waste or carousel from Kingfisher and the PCR plate is sealed.

The completion time is 4 hours for at least about 5 plates.

### Example 2

During the Post Amplification Plate preparation, the Pre Amplification PCR plate is placed on the Inheco and the protocol is run. The PCR plate is centrifuged and peeled, moved to the Star and transferred to the new Kingfisher plate. The reagents are dispensed on the Certus dispenser and transferred to the Kingfisher. The wash plates are loaded, Kingfisher routine ran, and transferred to the Star. The QC plate and PCR plate are made. The beads are then added with Star, the Kingfisher routine ran, transferred to the Star, and 8 PCR plates are generated. The PCR protocol is then ran, the Ampure cleanup protocol is repeated on the Star and Kingfisher. The QC plate is made, ran on the fragment analyzer, and the output and pool samples on the Star are normalized.

### Example 3

The automated platform is used to isolate biomolecules from the biological sample and deliver for them for sequencing. The blood sample in a tube or one or more slices from an FFPE tumor biopsy is inserted into the system. During an initial quality control check, the amount of blood in the input tube is validated. The DNA from the blood sample or tumor biopsy is extracted from the white blood cells and the cell free DNA in the plasma.

During the quality check fragment analysis for the biological sample's DNA, the distribution size is 150 bp for the FFPE tumor fragment, 160 bp for the cell free fragment, and 20 kb for the buffy coat fragment. The isolated DNA has a concentration of 50 ng/uL for the buffy coat and 10 ng/uL for the FFPE tumor, and 100 pg/uL for the cell free DNA. The DNA concentration is then adjusted for storage.

During the DNA library preparations for downstream processes, the DNA fragments are modified. The fragments undergo a quality control fragment analysis by determining the distribution sizes (200 bp for buffy coat fragments and 150 bp for FFPE fragments) for the modified DNA fragments and quantifying fragments. The fragments concentrations are 50 ng/uL for FFPE and buffy coat and 20 ng/uL for cell free DNA.

During target capture, DNA is selected based on its match with table 1. After target capture, the distribution of the size for the DNA fragments and the amount of DNA isolated are measured. Then, the DNA is adjusted to the correct concentration of 30 ng/uL and each patient library is tagged with a specific barcode for downstream analysis.

### Example 4

TABLE 1
Genes for Biomarkers
BIOMARKERS FOR CELL-FREE DNA

| | | | | | | | |
|---|---|---|---|---|---|---|---|
| ABL1 | AKT1 | AKT2 | AKT3 | ALK | APC | AR | ARAF |
| ARID1A | ASXL1 | ATM | ATR | AURKA | AURKB | AURKC | BAP1 |
| BCL2 | BRAF | BRCA1 | BRCA2 | BRD2 | BRD3 | BRD4 | CCND1 |
| CCND2 | CCND3 | CCNE1 | CDH1 | CDK12 | CDK4 | CDK6 | CDKN1A |
| CDKN1B | CDKN2A | CDKN2B | CEBPA | CREBBP | CRKL | CSF1R | CTNNB1 |
| DDR2 | DNMT3A | EGFR | EPHA3 | EPHA5 | ERBB2 | ERBB3 | ERBB4 |
| ERCC2 | ERG | ERRFI1 | ESR1 | ETV1 | ETV4 | ETV5 | ETV6 |
| EWSR1 | EZH2 | FBXW7 | FGFR1 | FGFR2 | FGFR3 | FLCN | FLT3 |
| GATA3 | GNA11 | GNAQ | GNAS | GSTM1 | HNF1A | BRAS | IDH1 |
| IDH2 | IGF1R | JAK2 | JAK3 | KDR | KEAP1 | KIT | KMT2A |
| KRAS | MAP2K1 | MAP2K2 | MAP2K4 | MAPK1 | MAPK3 | MCL1 | MDM2 |
| MDM4 | MED12 | MEN1 | MET | MITF | MKI67 | MLH1 | MPL |
| MSH2 | MSH6 | MTOR | MYC | MYD88 | NF1 | NF2 | NFE2L2 |
| NFKBIA | NKX2-1 | NOTCH1 | NOTCH2 | NPM1 | NRAS | NTRK1 | NTRK3 |
| NUTM1 | PDGFRA | PDGFRB | PGR | PIK3CA | PIK3CB | PIK3R1 | PTCH1 |
| PTEN | PTPN11 | RAB35 | RAF1 | RARA | RB1 | RET | RHEB |
| RHOA | RIT1 | RNF43 | ROS1 | RSPO2 | RUNX1 | SMAD2 | SMAD4 |
| SMARCA4 | SMARCB1 | SMO | SRC | STK11 | SYK | TERT | TET2 |
| TMPRSS2 | TP53 | TSC1 | TSC2 | VHL | WT1 | XPO1 | ZNRF3 |
| BTK | CD274 | FOXL2 | MYCN | PDCD1LG2 | VEGFA | | |

EXON BIOMARKERS

| | | | | | | | |
|---|---|---|---|---|---|---|---|
| 61E3.4 | AAK1 | AARS | AARS2 | AATK | ABCB1 | ABCC9 | ABI1 |
| ABL1 | ABL2 | AC099552.4 | ACKR3 | ACP1 | ACSL3 | ACSL6 | ACSM2B |
| ACTA2 | ACTB | ACTC1 | ACTG1 | ACTL6B | ACTR2 | ACVR1 | ACVR1B |
| ACVR1C | ACVR2A | ACVR2B | ACVRL1 | ADAM10 | ADAM29 | ADAMTS10 | ADAMTS16 |
| ADAMTS2 | ADAMTS20 | ADCK1 | ADCK2 | ADCK3 | ADCK4 | ADCK5 | ADCY1 |
| ADORA2A | ADRB1 | ADRB2 | ADRBK1 | ADRBK2 | AES | AFAP1 | AFF1 |
| AFF3 | AFF4 | AGBL4 | AGXT2 | AHCTF1 | AHCYL2 | AHDC1 | AHNAK |
| AHNAK2 | AJUBA | AK9 | AKAP1 | AKAP13 | AKAP9 | AKR1B10 | AKT1 |
| AKT2 | AKT3 | AL603965.1 | ALDH2 | ALDH3A2 | ALDH7A1 | ALG10B | ALK |
| ALKBH2 | ALKBH3 | ALOX12B | ALOX5 | ALPK1 | ALPK2 | ALPK3 | AMER1 |
| AMHR2 | AMPH | ANAPC1 | ANKK1 | ANKRD11 | ANKRD12 | ANKRD20A4 | ANKRD30A |
| ANKRD36 | ANKRD53 | ANKRD6 | ANXA6 | ANXA8L2 | AP003733.1 | AP2A1 | APAF1 |
| APC | APC2 | APEX1 | APEX2 | API5 | APLF | APOB | APOBEC3G |
| APTX | AQP12A | AQP7 | AR | ARAF | AREG | ARFRP1 | ARG1 |
| ARG2 | ARHGAP26 | ARHGAP32 | ARHGAP35 | ARHGAP36 | ARHGEF12 | ARHGEF18 | ARHGEF35 |
| ARHGEF6 | ARID1A | ARID1B | ARID2 | ARID3A | ARID3B | ARID4A | ARID4B |
| ARID5A | ARID5B | ARNT | ASB5 | ASCL4 | ASH2L | ASPM | ASPSCR1 |
| ASTN2 | ASXL1 | ASXL2 | ASXL3 | ATF1 | ATF7IP | ATG13 | ATG5 |
| ATIC | ATM | ATP1A1 | ATP2B3 | ATR | ATRIP | ATRX | ATXN1 |
| AURKA | AURKB | AURKC | AXIN1 | AXIN2 | AXL | B2M | B3GNTL1 |
| B4GALT3 | BAGE2 | BAIAP2L1 | BAP1 | BARD1 | BAZ1B | BAZ2A | BBC3 |
| BCAP31 | BCKDK | BCL10 | BCL11A | BCL11B | BCL2 | BCL2A1 | BCL2L1 |
| BCL2L11 | BCL2L12 | BCL2L2 | BCL3 | BCL6 | BCL7A | BCL9 | BCL9L |
| BCLAF1 | BCOR | BCORL1 | BCR | BIRC2 | BIRC3 | BLK | BLM |
| BMP2K | BMPR1A | BMPR1B | BMPR2 | BMX | BPNT1 | BRAF | BRCA1 |
| BRCA2 | BRD2 | BRD3 | BRD4 | BRDT | BRINP3 | BRIP1 | BRSK1 |
| BRSK2 | BRWD3 | BTG1 | BTG2 | BTK | BUB1 | BUB1B | C11ORF30 |
| C15ORF65 | C16ORF59 | C19ORF40 | C1ORF159 | C1ORF86 | C1QTNF5 | C20ORF26 | C2CD3 |
| C2ORF44 | C3ORF70 | C4ORF27 | C7 | C7ORF50 | C7ORF55 | C8A | C8ORF37 |
| C8ORF44 | CABLES2 | CACNA1C | CACNA1D | CACNA1S | CAD | CALCR | CALM1 |
| CALN1 | CALR | CAMK1D | CAMK1G | CAMK2A | CAMK2B | CAMK2D | CAMK2G |
| CAMK4 | CAMKK1 | CAMKK2 | CAMKV | CAMTA1 | CANT1 | CARD11 | CARM1 |
| CARS | CASC5 | CASK | CASP8 | CAST | CBFA2T3 | CBFB | CBL |
| CBLB | CBLC | CBLN4 | CBWD1 | CCAR1 | CCDC107 | CCDC144A | CCDC160 |
| CCDC178 | CCDC6 | CCDC74A | CCNB1IP1 | CCND1 | CCND2 | CCND3 | CCNE1 |
| CCNH | CD163L1 | CD274 | CD276 | CD40 | CD5L | CD74 | CD79A |
| CD79B | CD82 | CDC14A | CDC14B | CDC20 | CDC25A | CDC25B | CDC25C |
| CDC27 | CDC42 | CDC42BPA | CDC42BPB | CDC42BPG | CDC42EP1 | CDC7 | CDC73 |
| CDH1 | CDH10 | CDH11 | CDH18 | CDH2 | CDH20 | CDH4 | CDH5 |
| CDH6 | CDH9 | CDK1 | CDK10 | CDK11A | CDK12 | CDK13 | CDK14 |
| CDK15 | CDK16 | CDK17 | CDK18 | CDK19 | CDK2 | CDK20 | CDK3 |
| CDK4 | CDK5 | CDK5RAP2 | CDK6 | CDK7 | CDK8 | CDK9 | CDKL1 |
| CDKL2 | CDKL3 | CDKL4 | CDKL5 | CDKN1A | CDKN1B | CDKN2A | CDKN2B |
| CDKN2C | CDKN3 | CDX2 | CEBPA | CEP170 | CEP89 | CETN2 | CFH |
| CFHR4 | CFLAR | CHAF1A | CHCHD7 | CHD2 | CHD3 | CHD4 | CHD5 |
| CHD7 | CHD8 | CHDC2 | CHEK1 | CHEK2 | CHIC2 | CHMP3 | CHN1 |
| CHUK | CIC | CIITA | CIT | CKMT1A | CKS1B | CLCN6 | CLDN18 |
| CLIP1 | CLK1 | CLK2 | CLK3 | CLK4 | CLP1 | CLSTN2 | CLTC |
| CLTCL1 | CLVS2 | CMKLR1 | CNBD1 | CNBP | CNOT1 | CNOT3 | CNPY3 |
| CNTN1 | CNTNAP5 | CNTRL | COBLL1 | COL11A1 | COL18A1 | COL1A1 | COL1A2 |
| COL2A1 | COL3A1 | COMT | COX6C | CPS1 | CPXCR1 | CR1 | CRB1 |
| CREB1 | CREB3L1 | CREB3L2 | CREBBP | CRIPAK | CRKL | CRLF2 | CRTC1 |
| CRTC3 | CSDE1 | CSF1 | CSF1R | CSF3R | CSK | CSNK1A1 | CSNK1A1L |
| CSNK1D | CSNK1E | CSNK1G1 | CSNK1G2 | CSNK1G3 | CSNK2A1 | CSNK2A2 | CTAGE6 |
| CTCF | CTDNEP1 | CTDSP1 | CTDSP2 | CTDSPL | CTDSPL2 | CTLA4 | CTNNA1 |
| CTNNA2 | CTNNB1 | CTNND1 | CTTN | CUL1 | CUL3 | CUX1 | CXCR4 |
| CYC1 | CYLD | CYP11B1 | CYP2A6 | CYP2B6 | CYP2C19 | CYP2C8 | CYP2C9 |
| CYP2D6 | CYP3A4 | CYP3A5 | CYP4F2 | DAB2IP | DACH1 | DACH2 | DAPK1 |
| DAPK2 | DAPK3 | DAXX | DCAF12L2 | DCC | DCLK1 | DCLK2 | DCLK3 |
| DCLRE1A | DCLRE1B | DCLRE1C | DCP1B | DCTN1 | DCUN1D1 | DDB1 | DDB2 |
| DDIT3 | DDR1 | DDR2 | DDX10 | DDX3X | DDX5 | DDX6 | DEFB114 |
| DEH3118 | DEFB119 | DEK | DERL1 | DHX16 | DHX9 | DIAPH1 | DICER1 |
| DIDO1 | DIO2 | DIS3 | DIS3L2 | DISP1 | DKK2 | DKK4 | DLG2 |
| DLX4 | DMC1 | DMD | DMPK | DNAH12 | DNAJA2 | DNAJC6 | DNER |
| DNM2 | DNM3 | DNMT1 | DNMT3A | DNMT3B | DOCK2 | DOCK4 | DOK6 |
| DOLPP1 | DOT1L | DPH3 | DPPA4 | DPYD | DRD2 | DRD5 | DSC2 |
| DSG2 | DSP | DST | DSTYK | DUPD1 | DUSP1 | DUSP10 | DUSP11 |
| DUSP12 | DUSP13 | DUSP14 | DUSP15 | DUSP16 | DUSP18 | DUSP19 | DUSP2 |
| DUSP21 | DUSP22 | DUSP23 | DUSP26 | DUSP27 | DUSP28 | DUSP3 | DUSP4 |
| DUSP5 | DUSP6 | DUSP7 | DUSP8 | DUSP9 | DUT | DYNC1I1 | DYRK1A |
| DYRK1B | DYRK2 | DYRK3 | DYRK4 | E2F3 | EBF1 | EBPL | ECT2L |
| EDNRB | EED | EEF1A1 | EEF2K | EGFL7 | EGFR | EGR3 | EIF1AX |
| EIF2AK1 | EIF2AK2 | EIF2AK3 | EIF2AK4 | EIF2S1 | EIF3E | EIF4A2 | ELAVL3 |
| ELF3 | ELF4 | ELF5 | ELK4 | ELL | ELN | ELTD1 | EME1 |
| EME2 | EMG1 | EML4 | ENDOV | EP300 | EPAS1 | EPB41L3 | EPCAM |
| EPDR1 | EPHA1 | EPHA10 | EPHA2 | EPHA3 | EPHA4 | EPHA5 | EPHA6 |
| EPHA7 | EPHA8 | EPHB1 | EPHB2 | EPHB3 | EPHB4 | EPHB6 | EPM2A |
| EPOR | EPPK1 | EPS15 | ERBB2 | ERBB2IP | ERBB3 | ERBB4 | ERC1 |
| ERCC1 | ERCC2 | ERCC3 | ERCC4 | ERCC5 | ERCC6 | ERCC6L | ERCC8 |
| ERG | ERN1 | ERN2 | ERRFI1 | ESPL1 | ESR1 | ESR2 | ESRRG |
| ETNK1 | ETS1 | ETV1 | ETV4 | ETV5 | ETV6 | EWSR1 | EXO1 |
| EXOSC10 | EXT1 | EXT2 | EYA1 | EYA2 | EYA3 | EYA4 | EZH1 |
| EZH2 | EZR | F2 | F5 | FADD | FAM101A | FAM129B | FAM129C |
| FAM131B | FAM155A | FAM157B | FAM174B | FAM175A | FAM194B | FAM21A | FAM46C |
| FAM46D | FAM58A | FAM71B | FAM83H | FAM86B1 | FAM86B2 | FAM9A | FAN1 |
| FANCA | FANCB | FANCC | FANCD2 | FANCE | FANCF | FANCG | FANCI |
| FANCL | FANCM | FANK1 | FAS | FASTK | FAT1 | FBN1 | FBN2 |
| FBXO11 | FBXO43 | FBXW7 | FCGR1A | FCGR2B | FCGR3B | FCHO2 | FCRL4 |
| FEN1 | FER | FES | FEV | FGF10 | FGF14 | FGF19 | FGF23 |
| FGF3 | FGF4 | FGF6 | FGF7 | FGFR1 | FGFR1OP | FGFR2 | FGFR3 |
| FGFR4 | FGR | FH | FHIT | FIP1L1 | FIS1 | FKBP9 | FLCN |
| FLI1 | FLNA | FLT1 | FLT3 | FLT4 | FN1 | FNBP1 | FOLR1 |
| FOSL2 | FOXA1 | FOXA2 | FOXL2 | FOXO1 | FOXO3 | FOXO4 | FOXP1 |
| FOXP4 | FOXQ1 | FRG1 | FRG2B | FRK | FRS2 | FSCN3 | FSIP1 |
| FSTL3 | FTH1 | FUBP1 | FUS | FUT9 | FYN | G3BP1 | G6PD |
| GAB2 | GAB3 | GABRA6 | GABRB2 | GABRB3 | GABRP | GAK | GALNT13 |
| GAS6 | GAS7 | GATA1 | GATA2 | GATA3 | GATA4 | GATA6 | GATS |
| GCK | GCSAML | GDI1 | GEN1 | GID4 | GIGYF2 | GIPC3 | GLA |
| GLI1 | GLI2 | GLIPR1L2 | GML | GMPS | GNA11 | GNA13 | GNAI1 |
| GNAQ | GNAS | GNL3L | GNPTAB | GOLGA2 | GOLGA5 | GOLGA6L6 | GOPC |
| GOT2 | GP6 | GPC3 | GPC6 | GPHN | GPR124 | GPR89A | GPRASP1 |
| GPS2 | GPSM1 | GREM1 | GRIN2A | GRIN3A | GRK4 | GRK5 | GRK6 |
| GRK7 | GRM3 | GRXCR1 | GSG2 | GSK3A | GSK3B | GSTM1 | GSTP1 |
| GSTT1 | GTF2H1 | GTF2H2 | GTF2H3 | GTF2H4 | GTF2H5 | GTF2I | GTF3C5 |
| GUCY1A2 | GUCY2C | GUCY2D | GUCY2F | H1F0 | H1FNT | H1FOO | H1FX |
| H2AFB1 | H2AFB2 | H2AFB3 | H2AFJ | H2AFV | H2AFX | H2AFY | H2AFY2 |
| H2AFZ | H2BFM | H2BFWT | H3F3A | H3F3B | H3F3C | HCK | HCN1 |
| HDAC1 | HDAC10 | HDAC11 | HDAC2 | HDAC3 | HDAC4 | HDAC5 | HDAC6 |
| HDAC7 | HDAC8 | HDAC9 | HDDC2 | HDHD1 | HDHD2 | HDHD3 | HECW1 |
| HELQ | HERC1 | HERC2 | HERPUD1 | HEY1 | HGF | HHLA2 | HIF1A |
| HIP1 | HIPK1 | HIPK3 | HIPK4 | HIST1H1A | HIST1H1B | HIST1H1C | HIST1H1D |
| HIST1H1E | HIST1H1T | HIST1H2AA | HIST1H2AB | HIST1H2AC | HIST1H2AD | HIST1H2AE | HIST1H2AG |
| HIST1H2AH | HIST1H2AI | HIST1H2AJ | HIST1H2AK | HIST1H2AL | HIST1H2AM | HIST1H2BA | HIST1H2BB |
| HIST1H2BC | HIST1H2BD | HIST1H2BE | HIST1H2BF | HIST1H2BG | HIST1H2BH | HIST1H2BI | HIST1H2BK |
| HIST1H2BL | HIST1H2BM | HIST1H2BO | HIST1H3A | HIST1H3B | HIST1H3C | HIST1H3D | HIST1H3F |
| HIST1H3G | HIST1H3H | HIST1H3I | HIST1H3J | HIST1H4A | HIST1H4B | HIST1H4C | HIST1H4D |
| HIST1H4E | HIST1H4F | HIST1H4G | HIST1H4I | HIST1H4J | HIST1H4K | HIST1H4L | HIST2H2AA3 |
| HIST2H2AA4 | HIST2H2AB | HIST2H2AC | HIST2H2BE | HIST2H3A | HIST2H3C | HIST2H3D | HIST2H4A |
| HIST3H2A | HIST3H2BB | HIST3H3 | HKR1 | HLA-A | HLA-B | HLF | HLTF |
| HMGA1 | HMGA2 | HMGXB4 | HNF1A | HNRNPA2B1 | HNRNPM | HOOK3 | HOXA11 |
| HOXA13 | HOXA3 | HOXA9 | HOXB13 | HOXC11 | HOXC13 | HOXD11 | HOXD13 |
| HPCAL4 | HRAS | HS6ST1 | HSD3B1 | HSP90AA1 | HSP90AA2P | HSP90AB1 | HSPA2 |
| HSPA5 | HSPA8 | HSPB8 | HUNK | HUS1 | HUWE1 | IAPP | IARS2 |
| ICK | ICOSLG | ID3 | IDH1 | IDH2 | IDO1 | IFNGR1 | IFNL3 |
| IFT172 | IGF1 | IGF1R | IGF2 | IGF2BP3 | IGF2R | IGH3P7 | IK |
| IKBKAP | IKBKB | IKBKE | IKBKG | IKZF1 | IKZF2 | IKZF3 | IL10 |
| IL18RAP | IL1RAPL1 | IL2 | IL21R | IL2RG | IL3 | IL32 | IL36A |
| IL6ST | IL7R | ILF2 | ILK | ILKAP | IMPA1 | IMPA2 | IMPAD1 |
| ING1 | INHBA | INPP1 | INPP4A | INPP4B | INPP5A | INPP5B | INPP5D |
| INPP5E | INPP5F | INPP5J | INPP5K | INPPL1 | INSR | INSRR | INTS1 |
| INTS4 | IRAK1 | IRAK2 | IRAK3 | IRAK4 | IRF2 | IRF4 | IRS1 |
| IRS2 | ISOC2 | ITGA6 | ITK | ITPA | ITPR1 | ITPR3 | JAK1 |
| JAK2 | JAK3 | JARID2 | JAZF1 | JMJD1C | JUN | KALRN | KANK3 |
| KAT6A | KAT6B | KCNE1 | KCNH2 | KCNJ11 | KCNJ5 | KCNQ1 | KCNT2 |
| KDM5A | KDM5B | KDM5C | KDM6A | KDM6B | KDR | KDSR | KEAP1 |
| KEL | KIAA1109 | KIAA1549 | KIAA1598 | KIDINS220 | KIF20B | KIF3A | KIF5B |
| KIFC3 | KIT | KLF4 | KLF5 | KLF6 | KLHL4 | KLHL6 | KLK2 |
| KLRG1 | KMT2A | KMT2B | KMT2C | KMT2D | KNSTRN | KRAS | KRT1 |
| KRTAP1-1 | KRTAP15-1 | KRTAP19-6 | KRTAP5-5 | KSR1 | KSR2 | KTN1 | LARS |
| LASP1 | LATS1 | LATS2 | LCE1B | LCK | LCP1 | LDLR | LEF1 |
| LENG9 | LEPR | LEPROTL1 | LGI4 | LHFP | LHPP | LHX9 | LIFR |
| LIG1 | LIG3 | LIG4 | LILRB5 | LIMK1 | LIMK2 | LIN28A | LIN28B |
| LIN7A | LMNA | LMO1 | LMO2 | LMOD2 | LMTK2 | LMTK3 | LPP |
| LPPR1 | LPPR2 | LPPR3 | LPPR4 | LPPR5 | LRFN5 | LRIG3 | LRP1B |
| LRP6 | LRRC4C | LRRC55 | LRRIQ1 | LRRIQ3 | LRRK1 | LRRK2 | LRRTM4 |
| LSM14A | LTBP1 | LTBR | LTK | LTV1 | LUC7L2 | LUM | LUZP2 |
| LYL1 | LYN | LZTR1 | MACF1 | MAD2L2 | MADCAM1 | MAF | MAFB |
| MAGEA3 | MAGEB18 | MAGEB2 | MAGEC1 | MAGI2 | MAK | MALT1 | MAML2 |
| MAP1A | MAP1B | MAP2K1 | MAP2K2 | MAP2K3 | MAP2K4 | MAP2K5 | MAP2K6 |
| MAP2K7 | MAP3K1 | MAP3K10 | MAP3K11 | MAP3K12 | MAP3K13 | MAP3K14 | MAP3K2 |
| MAP3K3 | MAP3K4 | MAP3K5 | MAP3K6 | MAP3K7 | MAP3K8 | MAP3K9 | MAP4 |
| MAP4K1 | MAP4K3 | MAP4K4 | MAP4K5 | MAPK1 | MAPK10 | MAPK11 | MAPK12 |
| MAPK13 | MAPK14 | MAPK15 | MAPK3 | MAPK4 | MAPK6 | MAPK7 | MAPK8 |
| MAPK8IP1 | MAPK9 | MAPKAPK2 | MAPKAPK3 | MAPKAPK5 | 2-Mar | MARCKSL1 | MARK1 |
| MARK2 | MARK3 | MARK4 | MAST1 | MAST2 | MAST3 | MAST4 | MASTL |
| MAT2A | MATK | MAX | MBD4 | MCL1 | MCM7 | MCTP1 | MDC1 |
| MDM2 | MDM4 | MDN1 | MECOM | MED12 | MED13 | MED16 | MED17 |
| MED20 | MEF2A | MEF2B | MEF2C | MEGF6 | MELK | MEN1 | MERTK |
| MET | METRNL | METTL14 | MGA | MGMT | MGRN1 | MICAL1 | MINPP1 |
| MITF | MKI67 | MKL1 | MKNK1 | MKNK2 | MKRN1 | MLF1 | MLH1 |
| MLH3 | MLKL | MLLT1 | MLLT10 | MLLT11 | MLLT3 | MLLT4 | MLLT6 |
| MME | MMP2 | MMP24 | MMP9 | MMS19 | MN1 | MNAT1 | MNX1 |
| MOK | MOS | MPG | MPL | MPLKIP | MPND | MPP7 | MPRIP |
| MRAS | MRE11A | MROH2B | MRPS31 | MRPS9 | MSH2 | MSH3 | MSH4 |
| MSH5 | MSH6 | MSI2 | MSMB | MSN | MST1 | MST1R | MST4 |
| MTCP1 | MTF2 | MTHFR | MTM1 | MTMR1 | MTMR10 | MTMR11 | MTMR12 |
| MTMR2 | MTMR3 | MTMR4 | MTMR6 | MTMR7 | MTMR8 | MTMR9 | MTOR |
| MTRNR2L1 | MTRNR2L8 | MTUS2 | MUC1 | MUC2 | MUC4 | MUC6 | MUC7 |
| MUM1L1 | MUS81 | MUSK | MUTYH | MYB | MYBL1 | MYBPC3 | MYC |
| MYCBP2 | MYCN | MYD88 | MYH11 | MYH7 | MYH9 | MYL10 | MYL2 |
| MYL3 | MYLK | MYLK2 | MYLK3 | MYLK4 | MYNN | MYO1D | MYO3A |
| MYO3B | MYO5A | MYOD1 | MYOZ3 | MYT1 | NAA15 | NAB2 | NABP2 |
| NACA | NACC2 | NALCN | NAP1L2 | NAT2 | NAV1 | NAV3 | NBEA |
| NBN | NBPF10 | NCF1 | NCKIPSD | NCOA1 | NCOA2 | NCOA3 | NCOA4 |
| NCOA7 | NCOR1 | NCOR2 | NDRG1 | NEB | NEDD4L | NEFH | NEIL1 |
| NEIL2 | NEIL3 | NEK1 | NEK10 | NEK11 | NEK2 | NEK3 | NEK4 |
| NEK5 | NEK6 | NEK7 | NEK8 | NEK9 | NELFA | NELFB | NF1 |
| NF2 | NFATC2 | NFE2L2 | NFE2L3 | NFIB | NFKB1 | NFKB2 | NFKBIA |
| NFKBIB | NFKBIE | NFKBIZ | NHEJ1 | NIM1 | NIN | NIPBL | NKX2-1 |
| NKX3-1 | NLK | NLRP2 | NLRP3 | NLRP5 | NLRP6 | NM | NMS |
| NMT2 | NOD1 | NOMO1 | NONO | NOTCH1 | NOTCH2 | NOTCH2NL | NOTCH3 |
| NOTCH4 | NPAS3 | NPEPL1 | NPEPPS | NPM1 | NPR1 | NPR2 | NQO1 |
| NR | NR1H2 | NR4A2 | NR4A3 | NRAS | NRBP1 | NRBP2 | NRG1 |
| NRG3 | NRK | NSD1 | NT5C2 | NTHL1 | NTM | NTNG1 | NTRK1 |
| NTRK2 | NTRK3 | NUAK1 | NUAK2 | NUDT1 | NUDT10 | NUDT11 | NUDT14 |
| NUDT3 | NUDT4 | NUMA1 | NUMBL | NUP214 | NUP93 | NUP98 | NUTM1 |
| NUTM2A | NUTM2B | NXPE1 | OBSCN | OCRL | OGG1 | OLIG2 | OMD |
| OR2L2 | OR2W3 | OR5L1 | OR9G1 | OSBPL6 | OSR1 | OTOL1 | OTUB1 |
| OTUD4 | OXA1L | OXNAD1 | OXR1 | P2RY11 | P2RY8 | P4HB | PABPC1 |
| PABPC3 | PABPC4 | PABPC5 | PACS1 | PADI2 | PADI4 | PAFAH1B2 | PAK1 |
| PAK2 | PAK3 | PAK4 | PAK6 | PAK7 | PALB2 | PAN3 | PAPD5 |
| PARK2 | PARM1 | PARP1 | PARP2 | PARP3 | PASK | PATZ1 | PAX3 |
| PAX5 | PAX7 | PAX8 | PBK | PBRM1 | PBX1 | PCBP1 | PCDH11X |
| PCK1 | PCM1 | PCMTD1 | PCNA | PCSK7 | PCSK9 | PDCD1 | PDCD1LG2 |
| PDE1A | PDE4DIP | PDGFB | PDGFRA | PDGFRB | PDIK1L | PDK1 | PDK2 |
| PDK3 | PDK4 | PDP2 | PDPK1 | PDS5A | PDS5B | PDXP | PDYN |
| PEAK1 | PEG3 | PER1 | PES1 | PFN2 | PGM5 | PGP | PGR |
| PHF1 | PHF19 | PHF6 | PHKG1 | PHKG2 | PHLDA1 | PHLDA3 | PHLPP2 |
| PHOX2B | PICALM | PIK3C2B | PIK3C2G | PIK3C3 | PIK3CA | PIK3CB | PIK3CD |
| PIK3CG | PIK3R1 | PIK3R2 | PIK3R3 | PIK3R4 | PIM1 | PIM2 | PIM3 |
| PINK1 | PIP5K1A | PJA1 | PKD1 | PKD2 | PKDCC | PKHD1 | PKN1 |
| PKN2 | PKN3 | PKP2 | PLAG1 | PLAGL1 | PLCG1 | PLCG2 | PLCH2 |
| PLCL1 | PLEC | PLEKHS1 | PLK1 | PLK2 | PLK3 | PLK4 | PMAIP1 |
| PML | PMS1 | PMS2 | PNCK | PNKP | PNLIPRP3 | PNRC1 | POLB |
| POLD1 | POLE | POLG | POLH | POLI | POLK | POLL | POLM |
| POLN | POLQ | POLR2D | POM121L12 | POMK | POT1 | POTEC | POTEF |
| POTEG | POU2AF1 | POU3F2 | POU5F1 | PPA1 | PPA2 | PPAP2A | PPAP2B |
| PPAP2C | PPAPDC1A | PPAPDC1B | PPAPDC2 | PPAPDC3 | PPARG | PPEF1 | PPEF2 |
| PPFIA4 | PPFIBP1 | PPIF | PPM1A | PPM1B | PPM1D | PPM1E | PPM1F |
| PPM1G | PPM1H | PPM1J | PPM1K | PPM1L | PPM1M | PPM1N | PPP1CA |
| PPP1CB | PPP1CC | PPP2CA | PPP2CB | PPP2R1A | PPP3CA | PPP3CB | PPP3CC |
| PPP4C | PPP5C | PPP6C | PPTC7 | PRB1 | PRB2 | PRB4 | PRCC |
| PRDM1 | PRDM16 | PRDM2 | PRELID2 | PREX2 | PRF1 | PRG4 | PRKAA1 |
| PRKAA2 | PRKACA | PRKACB | PRKACG | PRKAG2 | PRKAR1A | PRKAR1B | PRKCA |
| PRKCB | PRKCD | PRKCE | PRKCG | PRKCH | PRKCI | PRKCQ | PRKCZ |
| PRKD3 | PRKDC | PRKG1 | PRKG2 | PRKX | PRPF19 | PRPF4 | PRPF8 |
| PRRC2A | PRRX1 | PRSS1 | PRSS3 | PRSS8 | PRX | PSEN1 | PSG5 |
| PSG6 | PSG8 | PSIP1 | PSKH1 | PSKH2 | PSMD11 | PSME3 | PSPH |
| PTCH1 | PTCH2 | PTEN | PTH | PTK2 | PTK2B | PTK6 | PTK7 |
| PTP4A1 | PTP4A2 | PTP4A3 | PTPDC1 | PTPLA | PTPMT1 | PTPN1 | PTPN11 |
| PTPN12 | PTPN13 | PTPN14 | PTPN18 | PTPN2 | PTPN20A | PTPN21 | PTPN22 |
| PTPN23 | PTPN3 | PTPN4 | PTPN5 | PTPN6 | PTPN7 | PTPN9 | PTPRA |
| PTPRB | PTPRC | PTPRD | PTPRE | PTPRF | PTPRG | PTPRH | PTPRJ |
| PTPRK | PTPRM | PTPRN | PTPRN2 | PTPRO | PTPRQ | PTPRR | PTPRS |
| PTPRT | PTPRU | PTPRZ1 | PWP1 | PWWP2A | PXK | PXN | PYDC2 |
| QKI | RAB11FIP5 | RAB35 | RABEP1 | RAC1 | RAC2 | RAD1 | RAD17 |
| RAD18 | RAD21 | RAD23A | RAD23B | RAD50 | RAD51 | RAD51B | RAD51C |
| RAD51D | RAD52 | RAD54B | RAD54L | RAD9A | RAF1 | RAG1 | RAI14 |
| RALGAPA1 | RALGDS | RANBP17 | RANBP2 | RANBP3 | RANGAP1 | RAP1GDS1 | RARA |
| RASA1 | RB1 | RBBP8 | RBFOX2 | RBM10 | RBM11 | RBM15 | RBMX |
| RCN1 | RDM1 | RECQL | RECQL4 | RECQL5 | REG1A | REG1B | REG3A |
| REG3G | REL | RELA | RELB | RERE | RERG | RET | REV1 |
| REV3L | RFWD2 | RGPD8 | RGS18 | RHEB | RHOA | RHOB | RHOH |
| RHOT1 | RICTOR | RIF1 | RIMS2 | RIOK1 | RIOK2 | RIOK3 | RIPK1 |
| RIPK2 | RIPK3 | RIPK4 | RIT1 | RMI2 | RNASEL | RNF10 | RNF111 |
| RNF144A | RNF168 | RNF185 | RNF213 | RNF34 | RNF4 | RNF43 | RNF8 |
| RNGTT | ROBO3 | ROCK1 | ROCK2 | ROR1 | ROR2 | ROS1 | RP11- |
| | | | | | | | 160N1.10 |
| RP11-181C3.1 | RP11- | RP11-758M4.1 | RPA1 | RPA2 | RPA3 | RPA4 | RPGR |
| | 683L23.1 | | | | | | |
| RPL10 | RPL10L | RPL13A | RPL22 | RPL5 | RPN1 | RPP38 | RPS27 |
| RPS6KA1 | RPS6KA2 | RPS6KA3 | RPS6KA4 | RPS6KA5 | RPS6KA6 | RPS6KB1 | RPS6KB2 |
| RPS6KC1 | RPS6KL1 | RPTOR | RQCD1 | RRAD | RRAS | RRAS2 | RRM1 |
| RRM2B | RSPO2 | RSPO3 | RSRC1 | RUNDC3B | RUNX1 | RUNX1T1 | RUNX2 |
| RXRA | RYBP | RYK | RYR1 | RYR2 | SACM1L | SAMHD1 | SATB2 |
| SAV1 | SBDS | SBF1 | SBF2 | SBK1 | SBK2 | SBK3 | SCN5A |
| SCYL1 | SCYL2 | SCYL3 | SDC4 | SDHA | SDHAF2 | SDHB | SDHC |
| SDHD | SEC23B | SEC31A | SECISBP2 | SEMA3C | SEMA3E | SEMG1 | SEPT5 |
| SEPT6 | SEPT9 | SERPINB3 | SERPINB4 | SET | SETBP1 | SETD2 | SETDB1 |
| SETDB2 | SETMAR | SETX | SF3B1 | SFPQ | SFRP1 | SGK1 | SGK2 |
| SGK223 | SGK3 | SGK494 | SGPP1 | SGPP2 | SH2B3 | SH2D1A | SH3GL1 |
| SH3PXD2A | SHFM1 | SHH | SHOC2 | SHPRH | SHQ1 | SI | SIK1 |
| SIK2 | SIK3 | SIN3A | SIRT1 | SIRT2 | SIRT3 | SIRT4 | SIRT5 |
| SIRT6 | SIRT7 | SKI | SKP2 | SLC12A2 | SLC13A1 | SLC17A8 | SLC1A2 |
| SLC22A13 | SLC25A10 | SLC25A4 | SLC25A5 | SLC26A3 | SLC34A2 | SLC38A4 | SLC3A2 |
| SLC45A3 | SLC5A7 | SLC9B1 | SLCO1B1 | SLIT2 | SLITRK6 | SLK | SLX1A |
| SLX1B | SLX4 | SMAD2 | SMAD3 | SMAD4 | SMARCA2 | SMARCA4 | SMARCAD1 |
| SMARCB1 | SMARCD1 | SMARCE1 | SMC1A | SMC3 | SMC4 | SMCHD1 | SMG1 |
| SMG7 | SMO | SMUG1 | SMYD4 | SNAP91 | SNCAIP | SND1 | SNRK |
| SNTG2 | SNX29 | SNX31 | SOCS1 | SOS1 | SOS2 | SOX10 | SOX17 |
| SOX2 | SOX9 | SP2 | SPAG16 | SPANXN1 | SPANXN2 | SPATA6 | SPECC1 |
| SPEG | SPEN | SPHKAP | SPNS1 | SPO11 | SPOCK3 | SPOP | SPRED1 |
| SPRR2G | SPRTN | SPRY1 | SPRY2 | SPRY4 | SPTA1 | SPTAN1 | SPTBN1 |
| SQSTM1 | SRC | SRCAP | SRCIN1 | SRGAP3 | SRM | SRPK1 | SRPK2 |
| SRPK3 | SRRM2 | SRSF2 | SRSF3 | SS18 | SS18L1 | SSH1 | SSH2 |
| SSH3 | SSX1 | SSX2 | SSX2IP | SSX4 | STAG1 | STAG2 | STAG3 |
| STARD6 | STAT3 | STAT4 | STAT5B | STAT6 | STEAP4 | STIL | STIP1 |
| STK10 | STK11 | STK16 | STK17A | STK17B | STK19 | STK24 | STK25 |
| STK3 | STK31 | STK32A | STK32B | STK32C | STK33 | STK35 | STK36 |
| STK38L | STK39 | STK40 | STRADA | STRADB | STRN | STYK1 | STYX |
| STYXL1 | SUFU | SULT1A1 | SULT1B1 | SUPT4H1 | SUPT5H | SUZ12 | SV2C |
| SVIL | SWI5 | SYK | SYNE1 | SYNJ1 | SYNJ2 | SYT4 | TAB1 |
| TACC1 | TADA1 | TADA2B | TAF1 | TAF15 | TAF1A | TAF1L | TAL1 |
| TANC2 | TAOK1 | TAOK2 | TAOK3 | TAS2R10 | TAS2R13 | TAS2R14 | TAS2R43 |
| TAS2R60 | TBC1D2B | TBC1D31 | TBCK | TBK1 | TBL1XR1 | TBP | TBX15 |
| TBX22 | TBX3 | TCEA1 | TCF12 | TCF3 | TCF4 | TCF7 | TCF7L2 |
| TCL1A | TDG | TDP1 | TDP2 | TEC | TECRL | TEK | TENC1 |
| TENM3 | TERT | TESK1 | TESK2 | TET1 | TET2 | TEX13A | TEX14 |
| TFDP1 | TFE3 | TFEB | TFG | TFPT | TFRC | TGEBR1 | TGEBR2 |
| TGIF1 | TGIF2LX | TGOLN2 | THADA | THEM5 | THEMIS | THRAP3 | TICAM1 |
| TIE1 | TIMM50 | TJP2 | TLK1 | TLK2 | TLR4 | TLX1 | TLX3 |
| TMCO5A | TMED4 | TMEM101 | TMEM127 | TMEM43 | TMPRSS2 | TMTC1 | TNC |
| TNFAIP3 | TNFRSF10C | TNFRSF11A | TNFRSF13B | TNFRSF14 | TNFRSF17 | TNIK | TNK1 |
| TNK2 | TNKS | TNKS1BP1 | TNKS2 | TNNI3 | TNNI3K | TNNT2 | TNPO1 |
| TNS1 | TNS3 | TOB2 | TOM1 | TOP1 | TOP2A | TOP3A | TOPBP1 |
| TP53 | TP53BP1 | TP53RK | TP53TG3D | TP63 | TPM1 | TPM3 | TPM4 |
| TPMT | TPR | TPSAB1 | TPSB2 | TPST1 | TPTE | TPTE2 | TRADD |
| TRAF2 | TRAF3 | TRAF7 | TRAT1 | TRDN | TREX1 | TREX2 | TRIM24 |
| TRIM27 | TRIM28 | TRIM33 | TRIM58 | TRIM7 | TRIML2 | TRIO | TRIP11 |
| TRMT10C | TRPM1 | TRPM3 | TRPM4 | TRPM6 | TRPM7 | TRPV4 | TRRAP |
| TSC1 | TSC2 | TSHR | TSHZ2 | TSHZ3 | TSPAN19 | TSSK1B | TSSK2 |
| TSSK3 | TSSK4 | TSSK6 | TTBK1 | TTBK2 | TTK | TTL | TTN |
| TUBA1A | TUSC3 | TWF1 | TWF2 | TXK | TXNIP | TYK2 | TYMS |
| TYRO3 | U2AF1 | UBALD1 | UBE2A | UBE2B | UBE2N | UBE2NL | UBE2V2 |
| UBE2Z | UBE4A | UBLCP1 | UBR5 | UBXN11 | UGT1A1 | UGT1A7 | UGT2A3 |
| UGT2B28 | UHMK1 | UHRF1BP1L | ULK1 | ULK2 | ULK3 | ULK4 | UNG |
| UQCRFS1 | USP2 | USP28 | USP29 | USP6 | USP7 | USP9X | UTP14A |
| UTY | UVSSA | VAT1L | VCPIP1 | VCX2 | VEGFA | VEGFC | VEZF1 |
| VEZT | VHL | VKORC1 | VRK1 | VRK2 | VRK3 | VTCN1 | VTI1A |
| WAPAL | WAS | WBSCR17 | WDR49 | WDR52 | WDR74 | WEE1 | WEE2 |
| WHSC1 | WHSC1L1 | WIF1 | WISP3 | WNK1 | WNK2 | WNK3 | WNK4 |
| WNT2 | WRN | WT1 | WWTR1 | XAB2 | XBP1 | XIAP | XPA |
| XPC | XPO1 | XPOT | XRCC1 | XRCC2 | XRCC3 | XRCC4 | XRCC5 |
| XRCC6 | YAP1 | YARS | YES1 | YME1L1 | YPEL5 | YWHAE | ZAP70 |
| ZBBX | ZBTB16 | ZBTB2 | ZBTB7B | ZCCHC3 | ZCCHC8 | ZDHHC14 | ZDHHC16 |
| ZEB2 | ZFHX3 | ZEP36L1 | ZFP36L2 | ZFP41 | ZIC4 | ZMAT4 | ZMYM2 |
| ZMYM3 | ZMYM4 | ZMYND8 | ZNF100 | ZNF132 | ZNF208 | ZNF217 | ZNF268 |
| ZNF28 | ZNF300 | ZNF324 | ZNF331 | ZNF384 | ZNF429 | ZNF444 | ZNF451 |
| ZNF488 | ZNF492 | ZNF493 | ZNF521 | ZNF567 | ZNF598 | ZNF668 | ZNF676 |
| ZNF703 | ZNF705G | ZNF708 | ZNF716 | ZNF717 | ZNF727 | ZNF750 | ZNF799 |
| ZNF80 | ZNF804A | ZNF804B | ZNF812 | ZNF814 | ZNF844 | ZNF91 | ZNF98 |
| ZNF99 | ZNRF3 | ZPBP | ZRSR2 | ZSWIM2 | MYCL | MYCL | MLK4 |
| MLK4 | ZAK | FRG1B | FRG1B | TRBV5-4 | | | |

INTRON BIOMARKERS

| | | | | | | | |
|---|---|---|---|---|---|---|---|
| ALK | BRAF | BRD3 | BRD4 | EGFR | ERG | ETV1 | ETV4 |
| ETV5 | EWSR1 | FGFR1 | FGFR2 | FGFR3 | MET | NOTCH1 | NRG1 |
| NTRK1 | NTRK2 | NTRK3 | NUTM1 | PDGFRA | PDGFRB | PRKCA | PRKCB |
| RAF1 | RET | ROS1 | TMPRSS2 | | | | |

PROMOTER BIOMARKERS

| | | | | | | | |
|---|---|---|---|---|---|---|---|
| AC099552.4 | ADAMTS10 | AGBL4 | ANKRD30BL | ANKRD53 | AP003733.1 | AP2A1 | ARHGEF18 |
| ARHGEF35 | BCL2 | BCL2L11 | C16orf59 | C4orf27 | CABLES2 | CACNA1C | CBWD1 |
| CCDC107 | CDC20 | CDH18 | CHMP3 | COL11A1 | CYLD | CYP4F2 | DIO2 |
| DLG2 | DNAJA2 | EZH2 | FAM129C | FAM21A | FCGR3B | GALNT13 | GOLGA2 |
| GPR89A | GTF2I | GTF3C5 | HCN1 | HERC2 | HKR1 | IGFBP7 | INSR |
| ISOC2 | ITPR1 | KALRN | KLRG1 | LENG9 | LEPROTL1 | LTV1 | LUC7L2 |
| MAGEA3 | MASTL | MED16 | MEF2C | MGRN1 | MPND | MRPS9 | MTRNR2L1 |
| MTRNR2L8 | MYNN | MYOZ3 | NALCN | NCOA7 | NEK11 | NFKBIE | NPAS3 |
| NPEPPS | NXPE1 | OR2L2 | OR2W3 | OR9G1 | OXNAD1 | PACS1 | PADI4 |
| PAPD5 | PFN2 | PLEKHS1 | POLR2D | POU5F1B | PPAPDC1A | PRSS1 | RAI14 |
| RGPD8 | RNF185 | RNF34 | RPL13A | RPS27 | SECISBP2 | SLC12A2 | SMG1 |
| SMUG1 | SNTG2 | SP2 | STAG3 | STAG3L5P- | TBC1D2B | TBC1D31 | TCF3 |
| | | | | PVRIG2P- | | | |
| | | | | PILR | | | |
| TCL1A | TERT | TNK2 | TPM3 | TPSAB1 | TPSB2 | TPTE | TRBV5-4 |
| TRMT10C | TRPM4 | TRPV4 | VCPIP1 | WDR74 | ZDHHC16 | ZNF324 | ZNF488 |
| ZNF708 | ZNF716 | ZNF717 | ZNF727 | ZNF799 | | | |

OTHER BIOMARKERS

| | | | | | | | |
|---|---|---|---|---|---|---|---|
| ADGRG6 | ALG10B | BAT25 (MSI) | BAT26 (MSI) | BCL11B | BCL2 | BCL6 | BCL7A |
| C1orf159 | CALM1 | CTNNA2 | D17S250 | D2S123 | D5S346 | DHX16 | DLX4 |
| | | | (MSI) | (MSI) | (MSI) | | |
| DRD5 | EEF1A1 | FGF7 | FLI1 | FSCN3 | GNAS | GP6 | HPCAL4 |
| INPP4B | LRRC4C | MAP2K2 | MAT2A | METRNL | NR21 (MSI) | NR22 (MSI) | NR27 (MSI) |
| PES1 | PLCL1 | PRELID2 | RCN1 | TBC1D31 | TENM3 | TOB2 | TP53TG3D |
| XBP1 | ZFP41 | ZNF208 | | | | | |

### Example 5

### Bioinformatics Pipeline

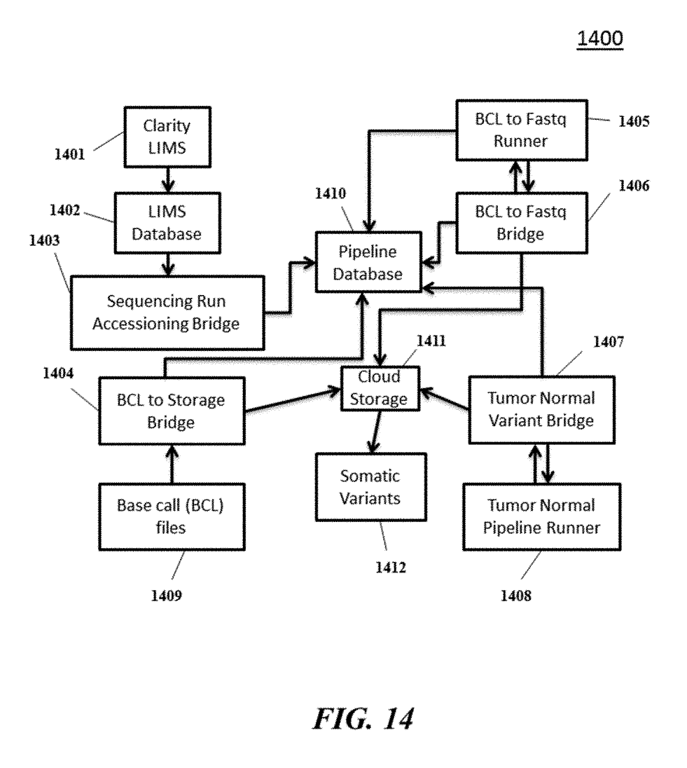

The bioinformatics pipeline uses raw sequencing data produced by NextSeq to identify multiple nucleotide variants, insertions or deletions of nucleotides, and copy number variants in a subject's biological sample. FIG. 14 shows an overview of the bioinformatics pipeline 1400. The language of the pipeline includes terms and phrases selected from the group consisting of user interface (UV), multiple nucleotide variant (MNV), copy number variant (CNV), insertion or deletion of nucleotides (Indel), variant call format (VCF), universally unique identifier (UUID), cloud storage service 1411, text file format used for storing sequenced reads (fastq file), database which stores the location and statuses for pipeline data (pipeline database 1410), and draft report (preliminary report). The preliminary report is received before the laboratory director's review and approval. The cloud storage service may be Google storage. The cloud storage service may be Amazon's S3 storage service (S3). The pipeline has two distinct steps. In the first step, sequencing run output is converted into FASTQ files. FASTQ files are represented in text file format for storing sequenced reads. Nest, sequencing runs are accessioned with the Clarity Laboratory Information System 1401 (Clarity LIMS). Information from the clarity LIMS is transferred to the LIMS data base 1402. The pipeline-bridge-service initiates the FASTQ conversion job in the Amazon cloud by running the bcl2fastq_runner. In the second step, the FASTQ files are used to identify somatic variants and copy number changes from matched normal and tumor sample pairs. The paired samples are accessioned by Clarity LIMS, which creates a case_id referencing one pair of normal sample fastq files, and one pair of tumor sample fastq files. The pipeline-bridge-service, known as tumor_normal_pipeline_runner, identifies somatic variants and copy number alterations using a proprietary algorithm.

The sequencing run accessioning bridge 1403 observes for new laboratory experiment metadata to be accessioned by the Clarity LIMS system, and stores the metadata into the pipeline database. The metadata allows the BCL2Fastq_runner to identify the method as to which sequencing libraries connect with sequencing runs and Illumina index adapters. The base call (BCL) to Storage Bridge 1404 (bcl2fastq) storage bridge observes the sequencing run output directory and, when the bridge identifies that a new sequencing run has finished, it can upload the BCL data into S3, and then insert the metadata about the sequencing run into the pipeline database. The BCL to Storage Bridge 1404 receives the NextSeq Output BCL files 1409. The BCL to FASTQ Bridge 1406 is responsible for running the bcl_to_fastq_runner conversion tool with the appropriate arguments, upload the newly generated FASTQ files into the pipeline database, and insert metadata into the pipeline database. The BCL to FASTQ runner 1405 converts the raw output of a sequencing run into fastq files in which reads are grouped by the sequencing library from which they originated. The case accessioning bridge links one library derived from a normal genomic sample to one derived from a tumor sample.

The tumor normal variant bridge 1407 can identifies cases for which the tumor/normal variant calling pipeline has not yet been run, and initiates a tumor normal pipeline runner 1408 instances for each of these cases. After the runs have finished (or failed), the tumor normal variant bridge updates the appropriate status fields in the pipeline database, sync the called variant data into S3, and update the database with the called variant files' locations. The tumor normal pipeline runner is responsible for identifying somatic variants 1412, such as multiple nucleotide variants, insertion or deletion of nucleotides, and identifying genes with significant copy number changes.

### Example 6

### DNA and cfDNA Assay

The DNA and cfDNA assays identify the presence and absence of molecular alterations (somatic mutations, copy number alterations, and fusion genes) involving the protein coding regions of the tumor DNA. This clinical report includes the approved drugs and drug candidates (i.e. drugs being studied in clinical trials), if any, that are associated with a potential clinical benefit or a potential lack of clinical benefit given the cancer-associated molecular alterations identified by the assays. The absence of a molecular alteration does not indicate necessarily that any drug or drug candidate will not provide any clinical benefit. Molecular alterations identified by the assay that are not associated with a potential clinical benefit or potential lack of clinical benefit is not listed in the report. The assay is performed using DNA derived from plasma and DNA derived from normal tissue. While germline DNA sequencing data is used for the identification of somatic mutations, germline events are not provided in the report. The somatic mutation, copy number alteration, and fusion detection portion of the assay is performed using the IDT xGen Lockdown system. Certain sample or variant characteristics may result in reduced sensitivity. These include but are not limited to low tumor cellularity, tumor heterogeneity, low mutant allele frequency, poor sample quality, and decreased fusion gene expression.

In an example, a subject with cancer submits his biological sample for DNA and cfDNA assaying for assessment of his molecular profile. In the DNA assay, the isolated genomic DNA derived from FFPE tumor tissue (QlAgen AllPrep DNA/RNA FFPE Kit) and matched normal tissue obtained from peripheral blood leukocytes (KingFisher Pure DNA Blood Kit) underwent sequencing library preparation using the KAPA HyperPrep Library Preparation kit. Prepared libraries were then target enriched using a customized version of the IDT xGen Lockdown system. Following enrichment, libraries for each sample were sequenced using the Illumina NextSeq 500 platform in order to generate at least 60 million, 75 bp paired-end reads with a mean target coverage of 450× for the tumor and 10 million reads with a mean target coverage of 70× for the normal samples. The tumor exome were sequenced to an average on-target depth of 450× and the matched normal tissues exome were sequenced to an average on-target depth of 70×.

Mutations, copy number variants, and fusions were screened for variants with strong clinical significance, variants with potential clinical significance, and variants with unknown significance. Variants with strong clinical significance were not identified in the subject. However, variants with potential clinical significance were identified including the AKT1 c.49G>A (p.E17K) mutation, ESR1 c.1609T>A (p.Y537N) mutation, ESR1 c.1273T>A (p.Y425N) mutation, ESR1 c.1609T>A (p.Y537N) mutation, and ESR1 c.826T>A (p.Y276N) mutation. Additionally, a copy number loss was detected for the subject's PGR gene. Lastly variants of unknown significance were identified including RERE c.472G>C (p.A158P), ASPM c.9621A>T (p. G3207G), ASPM c.4866A>T (p. G1622G), ASPM c.2616A>T (p. G872G), NAV1 c.3525G>A (p.R1175R), NAV1 c.3393G>A (p.R1131R), NAV1 c.3525G>A (p.R1175R), NAV1 c.3501G>A (p.R1167R), NAV1 c.3354G>A (p.R1118R), NAV1 c.2352G>A (p.R784R), NAV1 c.2172G>A (p.R724R), NAV1 c.471G>A (p.R157R), RANBP2 c.5910A>C (p.G1970G), NEB c.19633_19634insGGAAATATA (p.Y6545delinsWKYTKEQN), NEB c. 14530_1453 linsGGAAATATA (p.Y4844delinsWKYTKEQN), NEB c.3823_3824insGGAAATATACT (p.Y1275delinsWKYTKEQN), PTPRN c.966G>T (p.E322D), PTPRN c.696G>T (p.E232D), TNPO1 c.2621A>C (p.D874A), TNPO1 c.2471A>C (p.D874A), TNPO1 c.2597A>C (p.D866A), TNPO1 c.506A>C (p.D169A), ITPR3 c.5577G>A (p.Q1859Q), REV3L c.9359C>G (p.A3120G), REV3L c.9125C>G (p.A3042G), SYNE1 c.6787G>T (p.E2263*), SYNE1 c.6808G>T (p.E2270*), SYNE1 c.6898G>T (p.E2300*), DMD c.10262C>T (p.A3421V), DMD c.1058C>T (p.A353V), DMD c.2882C>T (p.A961V), DMD c.10250C>T (p.A3417V), DMD c.632C>T (p.A211V), HDAC6 c.1417G>A (p.E473K), and HDAC6 c.1375G>A (p.E459K). Copy number variants of unknown significance with gains in the copy number were identified.

In the cfDNA assay, the isolated cell-free DNA derived from plasma was obtained from the peripheral blood (MagMAX Cell-Free DNA Isolation Kit) and matched normal tissue was obtained from peripheral blood leukocytes (KingFisher Pure DNA Blood Kit). Next, both samples underwent sequencing library preparation using the Rubicon Genomics ThruPLEX Tag-seq Kit for cell-free DNA and the KAPA HyperPrep Library Preparation kit for normal DNA. Prepared libraries were target enriched using a customized version of the IDT xGen Lockdown system. Following enrichment, libraries for each samples were sequenced using the Illumina NextSeq 500 platform in order to generate at least a mean target coverage of 800X for the cell-free DNA library and 70× for the normal samples. The cell-free exome was sequenced to an average on-target depth of 800× and the matched normal tissues exome was sequenced to an average on-target depth of 70×.

Mutations and fusions were screened for variants with strong clinical significance, variants with potential clinical significance, and variants with unknown significance. Variants with strong clinical significance were not identified in the subject. However, the AKT1 c.49G>A (p.E17K) variant was identified as comprising with potential clinical significance and the APC c.3856G>T (p.E1286*) was identified as comprising unknown significance.

### Example 7

### Immunohistochemistry Assay

In another example, a subject with cancer submits his biological sample, which undergoes a molecular assessment using the immunohistochemistry assay. The assay reported a positive or negative score, an intensity score, a percentage of positivity, and a pass or no pass for the control. Upon obtaining a biological sample from the subject, the tissue was first fixed in 10% neutral buffered formalin for a minimum of at least 6 hours and a maximum of 72 hours. When detecting Estrogen Receptor (ER) or Progesterone Receptor (PR), the ER (clone SP1) and PR (clone 1E2) were diluted at a 1:1 ratio using Leica Bond Diluent. Next, slides were incubated for 30 minutes prior to following antigen retrieval with a citrate based buffer on the Leica Bond III. External controls with known intensity levels (1+, 2+and 3+) and with positive and negative punches were evaluated along with the test tissue. The control slides that are run alongside of the subject's sample showed the appropriate staining. ER and PR analysis was performed on the subject by immunohistochemistry utilizing the laboratory developed test (LDT). Interpretation of the ER and PR immuno-histochemical staining characteristics was guided by published results in the medical literature, information provided by the reagent manufacturer, and by internal review of staining performance. During interpretation of ER and PR, a positive result is reported when greater than 1% of the tumor cells show any nuclear staining. Contrarily, a negative result is reported when less than 1% of the tumor cells show any nuclear staining.

When detecting for the Human Epidermal Growth Factor Receptor 2 (HER2 Receptor), the HER2 Receptor (clone 4B5) was used as provided. Slides were incubated for 30 minutes prior to following antigen retrieval with a citrate based buffer on the Leica Bond III. External kit slides provided by the manufacturer (cells lines with 0, 1+, 2+and 3+expression) were evaluated along with the test tissue. The control slides run alongside of the subject's sample showed appropriate staining. HER2 analysis was performed on the subject by immunohistochemistry utilizing a LDT test. Interpretation of HER2 immuno-histochemical staining characteristics was guided by published results in the medical literature, information provided by the reagent manufacturer, and by internal review of staining performance. During interpretation of HER2, positive 3+ indicates a complete and circumferential membrane staining in greater than 10% of the tumor cells. Equivocal 2+ indicates circumferential membrane staining that is non-uniform and/or weak or moderate in greater than 10% of the tumor cells, or complete and circumferential membrane staining in 10% of the tumor cells. Negative 1+ indicates incomplete membrane staining that is faint and barely perceptible in greater than 10% of the tumor cells. Negative 0 indicates that there is no observable staining that is incomplete and faint or barely perceptible in 10% of the tumor cells. A HER2 2+staining result that is interpreted as equivocal may not show gene amplification. The results of the subject indicated a positive result with 3+ intensity score at 80% positive for the PR, negative result with 0 intensity score for the HER2, positive result with 3+ intensity score at 80% positive for the ER. All three passed the control test. When detecting for the Programmed Death-Ligand 1 (PD-L1), the PD-L1 (clone SP142, SP263, 22C3 and 28-8) was used as provided. Slides were incubated for 30 minutes prior to following antigen retrieval with an EDTA based buffer on the Leica Bond III. Control slides (cell lines with 0, 1+, 2+and 3+) were evaluated along with the test tissue. A batch negative reagent control was also used to test for non-specific binding. These control slides run alongside of the subject's sample showed appropriate staining. At least 100 tumor cells were identified for PD-L1 evaluation. PD-L1 analysis was performed on the subject by immune-histochemistry. Interpretation of PD-L1 immuno-histochemical staining characteristics was guided by published results in the medical literature, information provided by the reagent manufacturer, and by internal review of staining performance. The subject's PD-L1 immunohistochemistry results indicated a tumor proportion score of 8800 and immune cell score of 1800 for the 22C3 (Dako) and 28-8 (Dako) clones, a tumor proportion score of 0 and immune cell score of 0 for the SP263 (Ventana) clone, and a tumor proportion score of 800 and immune cell score of 1100 for the SP142 (Ventana) clone. All the clones passed the control test.

### Example 8

### Biologic Data and Medical History Record

In another example, the medical record of a subject was requested and then submitted for retrieval. Once obtained, records were checked for quality by examining legibility, completeness, and accuracy. Next, the records were inputted into the processing system and the resultant annotated medical record was attained. During processing, the records were cleaned, organized, and labeled. During labeling, the records were labeled according to relevant medical text segments. From the subject's documented medical records, the following description includes the list of topics that were identified as relevant in the processing of the subject's records and will be used for clinical trial matching. The medical terms and texts extracted from the subject's EHR were stored in a vector that is a representation of the subject's profile.

The subject's biologic data and medical history record as processed is reported below in Table 2. The biologic data and medical history record was processed into the label name, the label category, and the label value.

TABLE 2
Subject's Processed Biologic Data and Medical Record

| | | Label |
|---|---|---|
| Label Name | Label Category | Value |
| Is the patient diagnosed with breast | Diagnosis | Yes |
| cancer? | | |
| Does the patient currently have | Presentation Profile - | Yes |
| advanced or metastatic disease? | Disease and Metastases | |
| Does the patient currently have CNS | Presentation Profile - | Yes |
| metastases? | Disease and Metastases | |
| Has the patient ever received | Prior Therapies - | Yes |
| chemotherapy? | Chemotherapy | |
| Has the patient received radiation | Prior Therapies - | Yes |
| therapy? | Surgery or Radiation | |
| Is the patient HER2 positive? | Protein Expression | Yes |
| Is the patient HER2 negative? | Protein Expression | No |
| Is the patient female? | Medical History | Yes |
| Has the patient undergone a | Prior Therapies - | Yes |
| bilateral mastectomy? | Surgery or Radiation | |
| Has the patient received pertuzumab? | Prior Therapies - | Yes |
| | Targeted Therapy | |
| Has the patient received trastuzumab? | Prior Therapies - | Yes |
| | Targeted Therapy | |
| Has the patient received an aromatase | Prior Therapies - | Yes |
| inhibitor? | Hormone/Endocrine | |
| | Therapy | |
| Does the patient currently have CNS | Presentation Profile - | Yes |
| metastases? | Disease and Metastases | |
| Does the patient currently have | Presentation Profile - | Yes |
| advanced or metastatic disease? | Disease and Metastases | |
| Is the patient diagnosed with ductal | Diagnosis | Yes |
| breast cancer? | | |
| Is the patient ER+? | Protein Expression | Yes |
| Does the patient currently have a | Presentation Profile - | Maybe |
| condition that requires a prolonged | Medications | |
| use of steroids (exclude if <=10 mg | | |
| of prednisone)? | | |
| Is the patient ER+? | Protein Expression | Yes |
| Is the patient PR+? | Protein Expression | No |
| Has the patient received doxorubicin | Prior Therapies - | Yes |
| in the adjuvant or neoadjuvant | Chemotherapy | |
| setting? | | |
| Has the patient received paclitaxel | Prior Therapies - | Yes |
| in the adjuvant or neoadjuvant | Chemotherapy | |
| setting? | | |
| Has the patient received >=3 prior | Presentation Profile - | Maybe |
| lines of systemic anti-cancer therapy? | Number of Prior Anti- | |
| | Cancer Therapies | |
| Has the patient received >=2 prior | Presentation Profile - | Yes |
| lines of systemic anti-cancer therapy? | Number of Prior Anti- | |
| | Cancer Therapies | |
| Has the patient ever had a CNS | Oncologic History | Yes |
| metastasis? | | |
| Has the patient ever had multiple | Oncologic History | Yes |
| CNS metastatic lesions? | | |
| Does the patient currently have liver | Presentation Profile - | No |
| metastases? | Disease and Metastases | |
| Has the patient undergone a bilateral | Prior Therapies - | Yes |
| mastectomy? | Surgery or Radiation | |
| Has the patient undergone SLNB | Prior Therapies - | Yes |
| (Sentinel lymph node biopsy)? | Surgery or Radiation | |
| Is the patient diagnosed with ductal | Diagnosis | Yes |
| breast cancer? | | |
| Is the patient ER+? | Protein Expression | Yes |
| Is the patient diagnosed with breast | Diagnosis | Yes |
| cancer? | | |
| Does the patient currently have | Presentation Profile - | Yes |
| advanced or metastatic disease? | Disease and Metastases | |
| Does the patient currently have | Presentation Profile - | Yes |
| CNS metastases? | Disease and Metastases | |
| Has the patient undergone a | Prior Therapies - | Yes |
| bilateral mastectomy? | Surgery or Radiation | |
| Has the patient ever received | Prior Therapies - | Yes |
| chemotherapy? | Chemotherapy | |

### Example 9

### Clinical Trial Matching

In another example, the database of clinical trials is filtered according to phases of the clinical trial and according to eligibility by computer assessment based on a list of criteria. During eligibility assessment, one portion of the database of clinical trials is curated using one or more clinical labels and molecular labels to generate the filtered set of trials.

Next, the subject's medical history data and biologic data as reported in Examples 8 and 9 are collected. The medical history data and biologic data are computer analyzed to yield a genomic-based medical history analysis for the subject. The genomic-based medical history analysis is used to query the filtered list of eligible clinical trials for the subject to generate the subset of clinical trials for which the subject qualifies. First, ineligible therapies are determined according to a categorical score and rejected from the filtered list of therapies. The categorical score for each therapy is either a yes, maybe, or no. The categorical score may correspond to the group consisting of yes, maybe, and no. The therapies are then grouped using a similarity score between the subject and the therapies based on the labels. One similarity metric used is finding an empirical significance threshold and determining positive clinical trials by a specific criterion and then assessing overlap among positive clinical trials in a standard manner. The clinical trials that fall below a minimum similarity score for criteria crucial to trial enrollment are ineligible. Upon generation of the final list of therapies, the list is presented on a user interface on an electronic device of the subject. The subject will make a selection from the given therapies and will submit a request for enrollment. The list of therapies is also sent to a medically qualified staff member for final authorization and the clinical trials are added to the subject's profile.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for identifying a genomic aberration in one or more biological samples of a subject, comprising:
(a) obtaining said one or more biological samples of said subject, which one or more biological samples comprise a nucleic acid sample that has or is suspected of having one or more genomic aberration(s) that appears at a frequency of less than about 5% in said nucleic acid sample;
(b) enriching said nucleic acid sample for a plurality of nucleic acid sequences to provide an enriched nucleic acid sample using a probe set comprising probes that have an on-target rate as a group of at least about 80%, as determined by (i) measuring, for said probe set in at least one predetermined region, (1) probe coverage of each probe in said probe set and (2) off-target probe coverage for each probe in said probe set, and (ii) determining said on-target rate of said probe set based on a ratio of said off-target coverage to said probe coverage;
(c) sequencing said enriched nucleic acid sample to generate sequencing reads; and
(d) processing said sequencing reads to identify said genomic aberration(s) in said one or more biological samples of said subject that appears at a frequency of less than about 5% in said nucleic acid sample.

2. The method of claim 1, further comprising re-processing said one or more biological samples at a later point in time and identifying a change in one or more biological markers.

3. The method of claim 2, wherein said one or more biological makers are genes and variants selected from Table 1.

4. The method of claim 1, further comprising, prior to (a), receiving a request from said subject to process said one or more biological samples or sequence said one or more biological samples.

5. The method of claim 1, wherein (a)-(c) is performed without any involvement from a user during sample preparation.

6. The method of claim 1, wherein said genomic aberration(s) is identified at a concordance correlation coefficient of greater than or equal to about 90% and an accuracy of at least about 90% as compared to a control when said one or more biological samples is re-assayed for a presence or absence of said genomic aberration(s).

7. The method of claim 6, wherein greater than 90% of operations of (a)-(d) are automatically performed.

8. The method of claim 6, wherein said genomic aberration(s) is identified at a concordance correlation coefficient of greater than or equal to about 90% and an accuracy of at least about 90% based on assaying said one or more biological samples in at least two different geographic locations.

9. The method of claim 1, wherein said one or more biological samples is homogenous.

10. The method of claim 1, wherein said one or more biological samples is indexed.

11. The method of claim 1, wherein said one or more biological samples comprises a tumor tissue or a whole blood sample from said subject.

12. The method of claim 11, wherein said tumor tissue is a formalin-fixed, paraffin-embedded (FFPE) tissue.

13. The method of claim 1, wherein said one or more biological samples comprise normal biomolecules that are isolated from a buffy coat of said one or more biological samples.

14. The method of claim 1, wherein said one or more biological samples comprise abnormal biomolecules that are isolated from plasma or a tumor tissue of said one or more biological samples.

15. The method of claim 1, wherein said sequencing is selected from the group consisting of exome sequencing, transcriptome sequencing, and whole genome sequencing.

16. The method of claim 1, wherein said processing covers at least 2,500 genes, gene fusions, point mutations, indels, copy-number variations, promoters, or enhancers.

17. The method of claim 1, wherein said nucleic acid sample comprises cell-free DNA.

18. The method of claim 17, wherein said cell-free DNA molecules are sequenced using mismatch targeted sequencing or tethered elimination of termini sequencing.

19. The method of claim 1, further comprising using said genomic aberrations(s) identified in (d) to identify a disease in said subject.

20. The method of claim 1, wherein said genomic aberration(s) is identified using a plurality of biological markers from said one or more biological samples, which plurality of biological markers includes a plurality of different types of biological markers.

21. The method of claim 1, wherein said on-target rate as a group is at least about 90%,
